(19) United States
(12) Patent Application Publication
(10) Pub. No.: US 2008/0221185 A1
(43) Pub. Date: Sep. 11, 2008

(54) Glyt1 Transporter Inhibitors and Uses Thereof in Treatment of Neurological and Neuropsychiatric Disorders

(76) Inventors: Anthony William Dean, Hertfordshire (GB); Roderick Alan Porter, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/997,427

(22) PCT Filed: Jul. 31, 2006
(86) PCT No.: PCT/EP2006/007632

§ 371 Date: Jan. 31, 2008

(30) Foreign Application Priority Data
Aug. 2, 2005 (GB) 0515903.3
Mar. 16, 2006 (GB) 0605410.0

(51) Int. Cl. A61K 31/4184; C07D 235/02; A61P 25/18

(52) U.S. Cl. 514/387; 548/301.1

(57) ABSTRACT

The invention provides a compound of formula (I) or a solvate thereof:

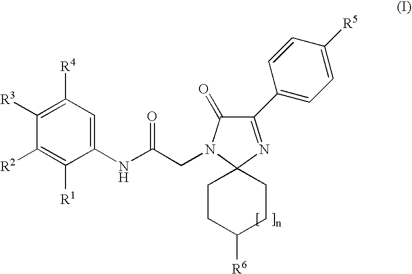

wherein R¹, R², R^{3,} R⁴, R⁵, R⁶, and n are as defined in the specification, and uses of such compounds. The compounds inhibit GlyT1 transporters and are useful in the treatment of certain neurological and neuropsychiatric disorders, including schizophrenia.

The present invention relates to glycine transporter inhibiting compounds, their use in the manufacture of medicaments for treating neurological and neuropsychiatric disorders, in particular psychoses, dementia or attention deficit disorder. The invention further comprises processes to make these compounds and pharmaceutical formulations thereof.

Molecular cloning has revealed the existence in mammalian brains of two classes of glycine transporters, termed GlyT1 and GlyT2. GlyT1 is found predominantly in the forebrain and its distribution corresponds to that of glutaminergic pathways and NMDA receptors (Smith, et al., Neuron, 8, 1992: 927-935). Molecular cloning has further revealed the existence of three variants of GlyT1, termed GlyT-Ia, GlyT-1b and GlyT-1c (Kim et al., Molecular Pharmacology, 45, 1994: 608-617), each of which displays a unique distribution in the brain and peripheral tissues. The variants arise by differential splicing and exon usage, and differ in their N-terminal regions. GlyT2, in contrast, is found predominantly in the brain stem and spinal cord, and its distribution corresponds closely to that of strychnine-sensitive glycine receptors (Liu et al., J. Biological Chemistry, 268, 1993: 22802-22808; Jursky and Nelson, J. Neurochemistry, 64, 1995 : 1026-1033). Another distinguishing feature of glycine transport mediated by GlyT2 is that it is not inhibited by sarcosine as is the case for glycine transport mediated by GlyT1. These data are consistent with the view that, by regulating the synaptic levels of glycine, GlyT1 and GlyT2 selectively influence the activity of NMDA receptors and strychnine-sensitive glycine receptors, respectively.

NMDA receptors are critically involved in memory and learning (Rison and Staunton, Neurosci. Biobehav. Rev., 19 533-552 (1995); Danysz et al, Behavioral Pharmacol., 6 455-474 (1995)); and, furthermore, decreased function of NMDA-mediated neurotransmission appears to underlie, or contribute to, the symptoms of schizophrenia (Olney and Farber, Archives General Psychiatry, 52, 998-1007 (1996). Thus, agents that inhibit GlyT1 and thereby increase glycine activation of NMDA receptors can be used as novel antipsychotics and anti-dementia agents, and to treat other diseases in which cognitive processes are impaired, such as attention deficit disorders and organic brain syndromes. Conversely, over-activation of NMDA receptors has been implicated in a number of disease states, in particular the neuronal death associated with stroke and possibly neurodegenerative diseases, such as Alzheimer's disease, multi-infarct dementia, AIDS dementia, Huntington's disease, Parkinson's disease, amyotrophic lateral sclerosis or other conditions in which neuronal cell death occurs, such as stroke or head trauma. Coyle & Puttfarcken, Science, 262, 689-695 (1993); Lipton and Rosenberg, New Engl. J. of Medicine, 330, 613-622 (1993); Choi, Neuron, 1, 623-634 (1988). Thus, pharmacological agents that increase the activity of GlyT1 will result in decreased glycine-activation of NMDA receptors, which activity can be used to treat these and related disease states. Similarly, drugs that directly block the glycine site of the NMDA receptors can be used to treat these and related disease states.

Glycine transport inhibitors are already known in the art, for example as disclosed in published international patent application WO03/055478 (SmithKline Beecham).

However, there still remains the need to identify further compounds that can inhibit GlyT1 transporters, including those that inhibit GlyT1 transporters selectively over GlyT2 transporters.

It has now been found that a novel class of compounds inhibit GlyT1 transporters and are thus useful in the treatment of certain neurological and neuropsychiatric disorders, including schizophrenia.

Thus, in the first aspect, there is provided a compound of formula (I) or a solvate thereof for use as a medicament:

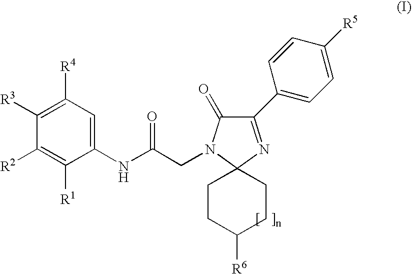

wherein:
R¹ is selected from H, methyl, methoxy and fluoro;
R² is selected from H, methyl, methoxy, fluoro and chloro;
R³ is selected from H, methyl, methoxy, fluoro and chloro;
R⁴ is selected from H and methoxy;
R⁵ is selected from H, methyl, methoxy, chloro and fluoro;
R⁶ is selected from H and methyl; and
n is selected from 0, 1 and 2;
excluding
N-[3,4-bis(methyloxy)phenyl]-2-(8-methyl-2-oxo-3-phenyl-1,4-diazaspiro[4.5]dec-3-en-1-yl)acetamide
2-(8-methyl-2-oxo-3-phenyl-1,4-diazaspiro[4.5]dec-3-en-1-yl)-N-[4-(methyloxy)phenyl]acetamide
2-[3-(4-fluorophenyl)-2-oxo-1,4-diazaspiro[4.4]non-3-en-1-yl]-N-(2-methylphenyl)acetamide
2-(2-oxo-3-phenyl-1,4-diazaspiro[4.4]non-3-en-1-yl)-N-phenylacetamide
N-(4-methylphenyl)-2-(2-oxo-3-phenyl-1,4-diazaspiro[4.4]non-3-en-1-yl)acetamide
N-[4-(methyloxy)phenyl]-2-(2-oxo-3-phenyl-1,4-diazaspiro[4.4]non-3-en-1-yl)acetamide
N-[3,4-bis(methyloxy)phenyl]-2-(2-oxo-3-phenyl-1,4-diazaspiro[4.4]non-3-en-1-yl)acetamide and
N-[3,5-bis(methyloxy)phenyl]-2-(2-oxo-3-phenyl-1,4-diazaspiro[4.4]non-3-en-1-yl)acetamide.

Compounds of formula (I) have been found to inhibit GlyT1 transporters.

In one embodiment, R¹ is selected from H, methyl and methoxy.

In one embodiment, R² is selected from H, methyl, fluoro, chloro and methoxy.

In one embodiment, R³ is selected from H, methyl, fluoro and chloro.

In a further embodiment, R³ is selected from H, methyl and fluoro.

In a further embodiment, R⁴ is methoxy.

In one aspect of the invention, n is selected from 1 and 2. In a further embodiment, n is 1.

In one embodiment, R⁵ is selected from H, methyl, methoxy, fluoro and chloro. In another embodiment, R⁵ is selected from methyl, methoxy and chloro. In another embodiment, R⁵ is selected from methoxy and chloro.

In a further embodiment, R⁶ is H.

For the avoidance of doubt, the embodiments of any one feature of the compounds of the invention may be combined with any embodiment of another feature of compounds of the invention to create a further embodiment.

As used herein, the term “solvate” refers to a complex of variable stoichiometry formed by a solute (in this invention, a compound of formula (I)) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include water, ethanol and acetic acid. Most preferably the solvent used is water.

Examples of compounds of the invention include:

| Structure | Name |
|---|---|
| 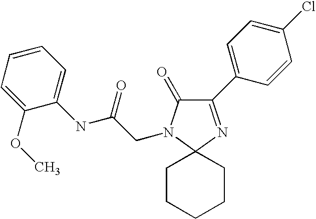 | 2-[3-(4-chlorophenyl)-2-oxo-1,4-diazaspiro[4.5]dec-3-en-1-yl]-N-[2-(methyloxy)phenyl]acetamide |
| 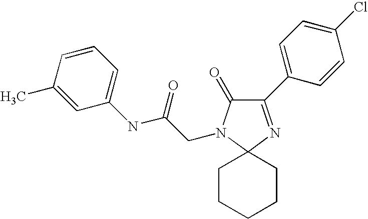 | 2-[3-(4-chlorophenyl)-2-oxo-1,4-diazaspiro[4.5]dec-3-en-1-yl]-N-(3-methylphenyl)acetamide |
| 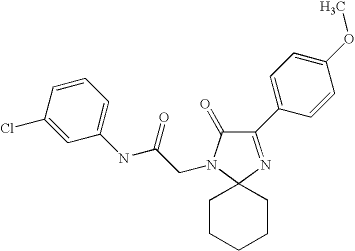 | N-(3-chlorophenyl)-2-{3-[4-(methyloxy)phenyl]-2-oxo-1,4-diazaspiro[4.5]dec-3-en-1-yl}acetamide |
| 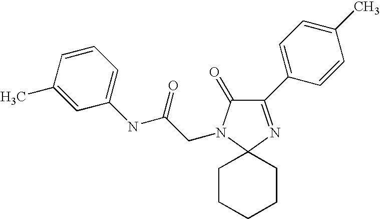 | N-(3-methylphenyl)-2-[3-(4-methylphenyl)-2-oxo-1,4-diazaspiro[4.5]dec-3-en-1-yl]acetamide |
| 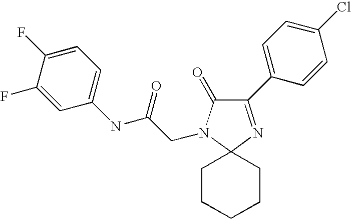 | 2-[3-(4-chlorophenyl)-2-oxo-1,4-diazaspiro[4.5]dec-3-en-1-yl]-N-(3,4-difluorophenyl)acetamide |
| 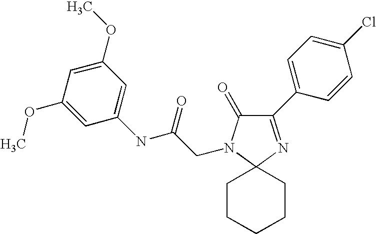 | N-[3,5-bis(methyloxy)phenyl]-2-[3-(4-chlorophenyl)-2-oxo-1,4-diazaspiro[4.5]dec-3-en-1-yl]acetamide |
| 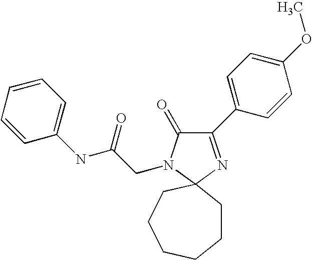 | 2-{3-[4-(methyloxy)phenyl]-2-oxo-1,4-diazaspiro[4.6]undec-3-en-1-yl}-N-phenylacetamide |
| 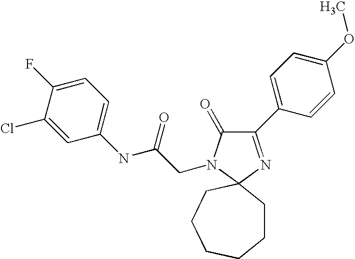 | N-(3-chloro-4-fluorophenyl)-2-{3-[4-(methyloxy)phenyl]-2-oxo-1,4-diazaspiro[4.6]undec-3-en-1-yl}acetamide |
| 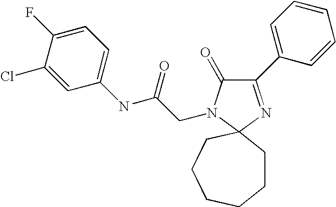 | N-(3-chloro-4-fluorophenyl)-2-(2-oxo-3-phenyl-1,4-diazaspiro[4.6]undec-3-en-1-yl)acetamide |
| 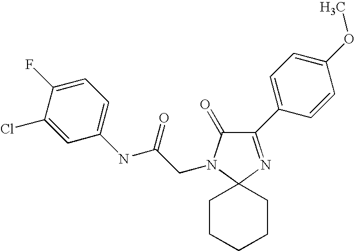 | N-(3-chloro-4-fluorophenyl)-2-{3-[4-(methyloxy)phenyl]-2-oxo-1,4-diazaspiro[4.5]dec-3-en-1-yl}acetamide |
| 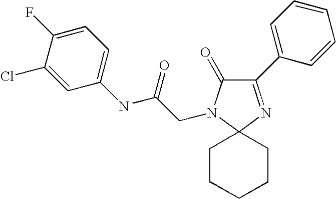 | N-(3-chloro-4-fluorophenyl)-2-(2-oxo-3-phenyl-1,4-diazaspiro[4.5]dec-3-en-1-yl)acetamide |
| 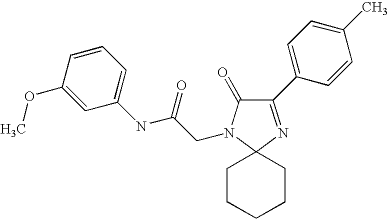 | N-[3-(methyloxy)phenyl]-2-[3-(4-methylphenyl)-2-oxo-1,4-diazaspiro[4.5]dec-3-en-1-yl]acetamide |
| 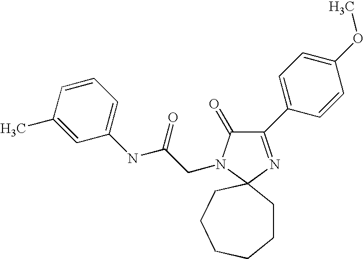 | 2-{3-[4-(methyloxy)phenyl]-2-oxo-1,4-diazaspiro[4.6]undec-3-en-1-yl}-N-(3-methylphenyl)acetamide |
| 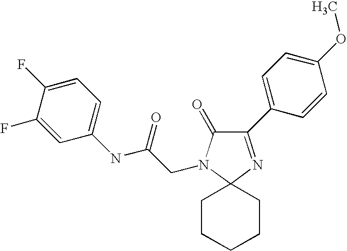 | N-(3,4-difluorophenyl)-2-{3-[4-(methyloxy)phenyl]-2-oxo-1,4-diazaspiro[4.5]dec-3-en-1-yl}acetamide |
| 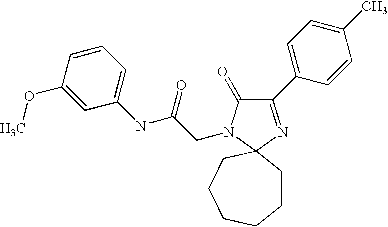 | N-[3-(methyloxy)phenyl]-2-[3-(4-methylphenyl)-2-oxo-1,4-diazaspiro[4.6]undec-3-en-1-yl]acetamide |
| 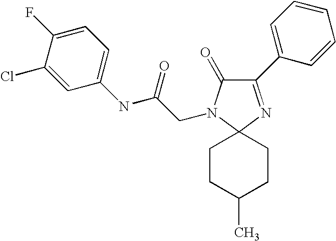 | N-(3-chloro-4-fluorophenyl)-2-(8-methyl-2-oxo-3-phenyl-1,4-diazaspiro[4.5]dec-3-en-1-yl)acetamide |
| 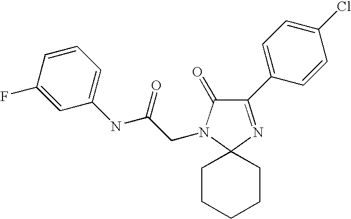 | 2-[3-(4-chlorophenyl)-2-oxo-1,4-diazaspiro[4.5]dec-3-en-1-yl]-N-(3-fluorophenyl)acetamide |
| 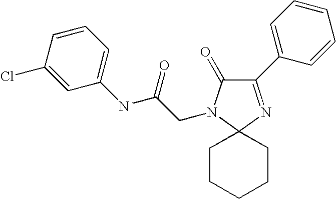 | N-(3-chlorophenyl)-2-(2-oxo-3-phenyl-1,4-diazaspiro[4.5]dec-3-en-1-yl)acetamide |
| 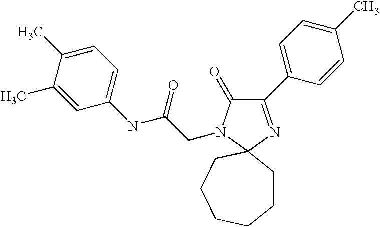 | N-(3,4-dimethylphenyl)-2-[3-(4-methylphenyl)-2-oxo-1,4-diazaspiro[4.6]undec-3-en-1-yl]acetamide |
| 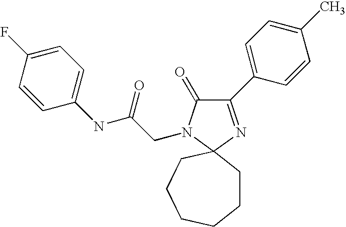 | N-(4-fluorophenyl)-2-[3-(4-methylphenyl)-2-oxo-1,4-diazaspiro[4.6]undec-3-en-1-yl]acetamide |
| 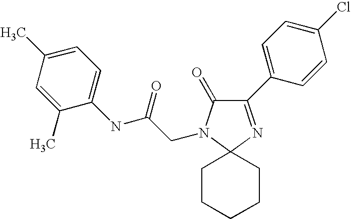 | 2-[3-(4-chlorophenyl)-2-oxo-1,4-diazaspiro[4.5]dec-3-en-1-yl]-N-(2,4-dimethylphenyl)acetamide |
| 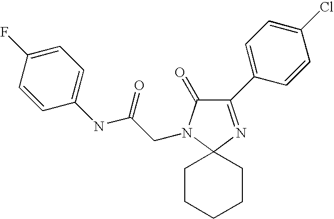 | 2-[3-(4-chlorophenyl)-2-oxo-1,4-diazaspiro[4.5]dec-3-en-1-yl]-N-(4-fluorophenyl)acetamide |
| 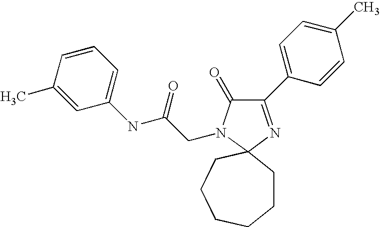 | N-(3-methylphenyl)-2-[3-(4-methylphenyl)-2-oxo-1,4-diazaspiro[4.6]undec-3-en-1-yl]acetamide |
| 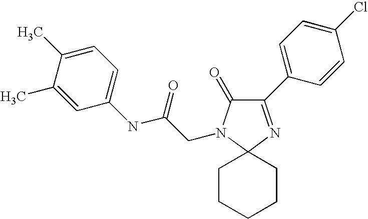 | 2-[3-(4-chlorophenyl)-2-oxo-1,4-diazaspiro[4.5]dec-3-en-1-yl]-N-(3,4-dimethylphenyl)acetamide |
| 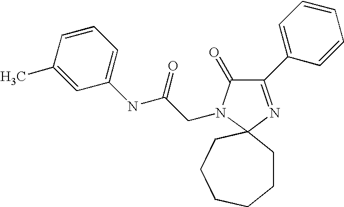 | N-(3-methylphenyl)-2-(2-oxo-3-phenyl-1,4-diazaspiro[4.6]undec-3-en-1-yl)acetamide |
| 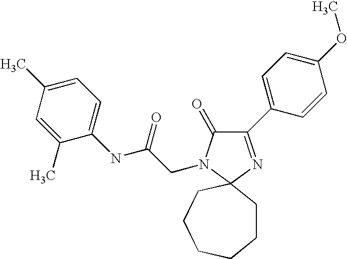 | N-(2,4-dimethylphenyl)-2-{3-[4-(methyloxy)phenyl]-2-oxo-1,4-diazaspiro[4.6]undec-3-en-1-yl}acetamide |
| 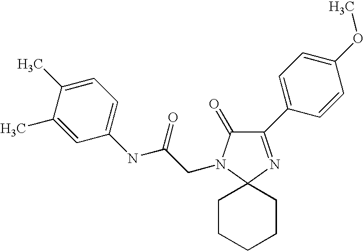 | N-(3,4-dimethylphenyl)-2-{3-[4-(methyloxy)phenyl]-2-oxo-1,4-diazaspiro[4.5]dec-3-en-1-yl}acetamide |
| 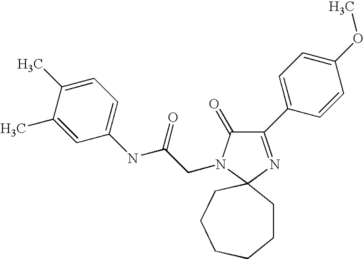 | N-(3,4-dimethylphenyl)-2-{3-[4-(methyloxy)phenyl]-2-oxo-1,4-diazaspiro[4.6]undec-3-en-1-yl}acetamide |
| 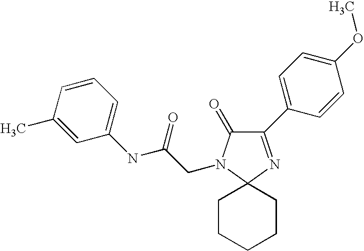 | 2-{3-[4-(methyloxy)phenyl]-2-oxo-1,4-diazaspiro[4.5]dec-3-en-1-yl}-N-(3-methylphenyl)acetamide |
| 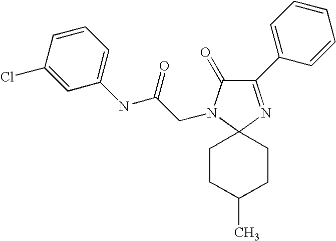 | N-(3-chlorophenyl)-2-(8-methyl-2-oxo-3-phenyl-1,4-diazaspiro[4.5]dec-3-en-1-yl)acetamide |
| 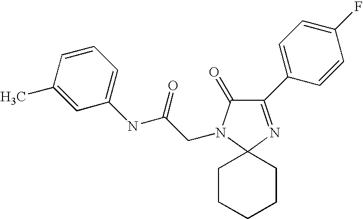 | 2-[3-(4-fluorophenyl)-2-oxo-1,4-diazaspiro[4.5]dec-3-en-1-yl]-N-(3-methylphenyl)acetamide |
| 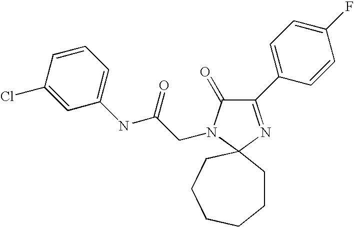 | N-(3-chlorophenyl)-2-[3-(4-fluorophenyl)-2-oxo-1,4-diazaspiro[4.6]undec-3-en-1-yl]acetamide |
| 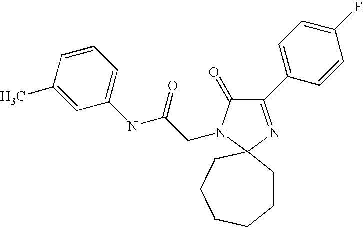 | 2-[3-(4-fluorophenyl)-2-oxo-1,4-diazaspiro[4.6]undec-3-en-1-yl]-N-(3-methylphenyl)acetamide |
| 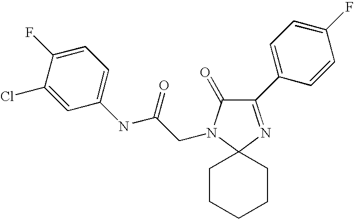 | N-(3-chloro-4-fluorophenyl)-2-[3-(4-fluorophenyl)-2-oxo-1,4-diazaspiro[4.5]dec-3-en-1-yl]acetamide |

2-(8-methyl-2-oxo-3-phenyl-1,4-diazaspiro[4.5]dec-3-en-1-yl)-N-phenylacetamide
N-(3-methylphenyl)-2-(2-oxo-3-phenyl-1,4-diazaspiro[4.5]dec-3-en-1-yl)acetamide
2-(8-methyl-2-oxo-3-phenyl-1,4-diazaspiro[4.5]dec-3-en-1-yl)-N-(3-methylphenyl)acetamide
N-(4-methylphenyl)-2-[3-(4-methylphenyl)-2-oxo-1,4-diazaspiro[4.5]dec-3-en-1-yl]acetamide
2-{3-[4-(methyloxy)phenyl]-2-oxo-1,4-diazaspiro[4.5]dec-3-en-1-yl}-N-(4-methylphenyl)acetamide
N-(3,4-dimethylphenyl)-2-(2-oxo-3-phenyl-1,4-diazaspiro[4.5]dec-3-en-1-yl)acetamide
N-(4-chlorophenyl)-2-(2-oxo-3-phenyl-1,4-diazaspiro[4.5]dec-3-en-1-yl)acetamide
N-(4-fluorophenyl)-2-(2-oxo-3-phenyl-1,4-diazaspiro[4.5]dec-3-en-1-yl)acetamide
N-(3,4-difluorophenyl)-2-(2-oxo-3-phenyl-1,4-diazaspiro[4.5]dec-3-en-1-yl)acetamide
2-[3-(4-chlorophenyl)-2-oxo-1,4-diazaspiro[4.5]dec-3-en-1-yl]-N-(2,4-difluorophenyl)acetamide
N-[3,5-bis(methyloxy)phenyl]-2-(8-methyl-2-oxo-3-phenyl-1,4-diazaspiro[4.5]dec-3-en-1-yl)acetamide
2-(2-oxo-3-phenyl-1,4-diazaspiro[4.5]dec-3-en-1-yl)-N-phenylacetamide
2-[3-(4-fluorophenyl)-2-oxo-1,4-diazaspiro[4.5]dec-3-en-1-yl]-N-(2-methylphenyl)acetamide
N-(2,3-dimethylphenyl)-2-[3-(4-fluorophenyl)-2-oxo-1,4-diazaspiro[4.5]dec-3-en-1-yl]acetamide
N-(4-chlorophenyl)-2-{3-[4-(methyloxy)phenyl]-2-oxo-1,4-diazaspiro[4.5]dec-3-en-1-yl}acetamide
N-(4-fluorophenyl)-2-{3-[4-(methyloxy)phenyl]-2-oxo-1,4-diazaspiro[4.5]dec-3-en-1-yl}acetamide
N-(3,4-difluorophenyl)-2-(8-methyl-2-oxo-3-phenyl-1,4-diazaspiro[4.5]dec-3-en-1-yl)acetamide
N-[3,5-bis(methyloxy)phenyl]-2-{3-[4-(methyloxy)phenyl]-2-oxo-1,4-diazaspiro[4.5]dec-3-en-1-yl}acetamide
N-(2,4-dimethylphenyl)-2-[3-(4-fluorophenyl)-2-oxo-1,4-diazaspiro[4.5]dec-3-en-1-yl]acetamide
N-[3-(methyloxy)phenyl]-2-{3-[4-(methyloxy)phenyl]-2-oxo-1,4-diazaspiro[4.5]dec-3-en-1-yl}acetamide
2-[3-(4-fluorophenyl)-2-oxo-1,4-diazaspiro[4.5]dec-3-en-1-yl]-N-[3-(methyloxy)phenyl]acetamide
N-[3,5-bis(methyloxy)phenyl]-2-[3-(4-methylphenyl)-2-oxo-1,4-diazaspiro[4.5]dec-3-en-1-yl]acetamide
N-(4-fluorophenyl)-2-[3-(4-fluorophenyl)-2-oxo-1,4-diazaspiro[4.5]dec-3-en-1-yl]acetamide
N-[4-(methyloxy)phenyl]-2-[3-(4-methylphenyl)-2-oxo-1,4-diazaspiro[4.5]dec-3-en-1-yl]acetamide
N-(4-fluorophenyl)-2-(8-methyl-2-oxo-3-phenyl-1,4-diazaspiro[4.5]dec-3-en-1-yl)acetamide
2-[3-(4-chlorophenyl)-2-oxo-1,4-diazaspiro[4.5]dec-3-en-1-yl]-N-[4-(methyloxy)phenyl]acetamide
N-[2,4-bis(methyloxy)phenyl]-2-[3-(4-fluorophenyl)-2-oxo-1,4-diazaspiro[4.5]dec-3-en-1-yl]acetamide
N-(4-methylphenyl)-2-(2-oxo-3-phenyl-1,4-diazaspiro[4.5]dec-3-en-1-yl)acetamide
N-(4-chlorophenyl)-2-[3-(4-fluorophenyl)-2-oxo-1,4-diazaspiro[4.5]dec-3-en-1-yl]acetamide
2-[3-(4-fluorophenyl)-2-oxo-1,4-diazaspiro[4.5]dec-3-en-1-yl]-N-[2-(methyloxy)phenyl]acetamide
N-(3,4-dimethylphenyl)-2-[3-(4-fluorophenyl)-2-oxo-1,4-diazaspiro[4.5]dec-3-en-1-yl]acetamide
N-[3-chloro-4-(methyloxy)phenyl]-2-(8-methyl-2-oxo-3-phenyl-1,4-diazaspiro[4.5]dec-3-en-1-yl)acetamide
2-{3-[4-(methyloxy)phenyl]-2-oxo-1,4-diazaspiro[4.5]dec-3-en-1-yl}-N-phenylacetamide
N-[3-(methyloxy)phenyl]-2-(2-oxo-3-phenyl-1,4-diazaspiro[4.5]dec-3-en-1-yl)acetamide
2-(2-oxo-3-phenyl-1,4-diazaspiro[4.6]undec-3-en-1-yl)-N-phenylacetamide
N-(4-methylphenyl)-2-(2-oxo-3-phenyl-1,4-diazaspiro[4.6]undec-3-en-1-yl)acetamide
2-{3-[4-(methyloxy)phenyl]-2-oxo-1,4-diazaspiro[4.6]undec-3-en-1-yl}-N-(4-methylphenyl)acetamide
N-(3,4-dimethylphenyl)-2-(2-oxo-3-phenyl-1,4-diazaspiro[4.6]undec-3-en-1-yl)acetamide
N-(4-chlorophenyl)-2-(2-oxo-3-phenyl-1,4-diazaspiro[4.6]undec-3-en-1-yl)acetamide
N-(4-chlorophenyl)-2-{3-[4-(methyloxy)phenyl]-2-oxo-1,4-diazaspiro[4.6]undec-3-en-1-yl}acetamide
N-(4-fluorophenyl)-2-(2-oxo-3-phenyl-1,4-diazaspiro[4.6]undec-3-en-1-yl)acetamide
N-(3-fluorophenyl)-2-(2-oxo-3-phenyl-1,4-diazaspiro[4.6]undec-3-en-1-yl)acetamide
N-(3,4-difluorophenyl)-2-(2-oxo-3-phenyl-1,4-diazaspiro[4.6]undec-3-en-1-yl)acetamide
N-[4-(methyloxy)phenyl]-2-(2-oxo-3-phenyl-1,4-diazaspiro[4.6]undec-3-en-1-yl)acetamide
N-[3,4-bis(methyloxy)phenyl]-2-(2-oxo-3-phenyl-1,4-diazaspiro[4.6]undec-3-en-1-yl)acetamide
N-[3-chloro-4-(methyloxy)phenyl]-2-(2-oxo-3-phenyl-1,4-diazaspiro[4.6]undec-3-en-1-yl)acetamide
N-[3-(methyloxy)phenyl]-2-(2-oxo-3-phenyl-1,4-diazaspiro[4.6]undec-3-en-1-yl)acetamide
N-[3,5-bis(methyloxy)phenyl]-2-(2-oxo-3-phenyl-1,4-diazaspiro[4.6]undec-3-en-1-yl)acetamide
N-[3,5-bis(methyloxy)phenyl]-2-[3-(4-methylphenyl)-2-oxo-1,4-diazaspiro[4.6]undec-3-en-1-yl]acetamide
N-[3-chloro-4-(methyloxy)phenyl]-2-[3-(4-methylphenyl)-2-oxo-1,4-diazaspiro[4.6]undec-3-en-1-yl]acetamide
N-(4-chlorophenyl)-2-[3-(4-methylphenyl)-2-oxo-1,4-diazaspiro[4.6]undec-3-en-1-yl]acetamide
N-(3-chloro-4-fluorophenyl)-2-[3-(4-methylphenyl)-2-oxo-1,4-diazaspiro[4.6]undec-3-en-1-yl]acetamide
N-(3-chlorophenyl)-2-{3-[4-(methyloxy)phenyl]-2-oxo-1,4-diazaspiro[4.6]undec-3-en-1-yl}acetamide
N-(2,3-dimethylphenyl)-2-[3-(4-fluorophenyl)-2-oxo-1,4-diazaspiro[4.6]undec-3-en-1-yl]acetamide
N-(3-chloro-4-fluorophenyl)-2-[3-(4-fluorophenyl)-2-oxo-1,4-diazaspiro[4.6]undec-3-en-1-yl]acetamide
2-[3-(4-fluorophenyl)-2-oxo-1,4-diazaspiro[4.6]undec-3-en-1-yl]-N-[2-(methyloxy)phenyl]acetamide
N-(4-methylphenyl)-2-[3-(4-methylphenyl)-2-oxo-1,4-diazaspiro[4.6]undec-3-en-1-yl]acetamide
2-[3-(4-fluorophenyl)-2-oxo-1,4-diazaspiro[4.6]undec-3-en-1-yl]-N-(2-methylphenyl)acetamide
N-[3-chloro-4-(methyloxy)phenyl]-2-[3-(4-fluorophenyl)-2-oxo-1,4-diazaspiro[4.6]undec-3-en-1-yl]acetamide
2-[3-(4-fluorophenyl)-2-oxo-1,4-diazaspiro[4.6]undec-3-en-1-yl]-N-(4-methylphenyl)acetamide
N-(4-chlorophenyl)-2-[3-(4-fluorophenyl)-2-oxo-1,4-diazaspiro[4.6]undec-3-en-1-yl]acetamide
N-[2,4-bis(methyloxy)phenyl]-2-[3-(4-fluorophenyl)-2-oxo-1,4-diazaspiro[4.6]undec-3-en-1-yl]acetamide
N-(3,4-dimethylphenyl)-2-[3-(4-fluorophenyl)-2-oxo-1,4-diazaspiro[4.6]undec-3-en-1-yl]acetamide
N-(4-fluorophenyl)-2-[3-(4-fluorophenyl)-2-oxo-1,4-diazaspiro[4.6]undec-3-en-1-yl]acetamide
2-[3-(4-fluorophenyl)-2-oxo-1,4-diazaspiro[4.6]undec-3-en-1-yl]-N-[3-(methyloxy)phenyl]acetamide
N-(2,4-dimethylphenyl)-2-[3-(4-fluorophenyl)-2-oxo-1,4-diazaspiro[4.6]undec-3-en-1-yl]acetamide
N-[3,5-bis(methyloxy)phenyl]-2-[3-(4-fluorophenyl)-2-oxo-1,4-diazaspiro[4.6]undec-3-en-1-yl]acetamide
N-(3,4-dimethylphenyl)-2-(2-oxo-3-phenyl-1,4-diazaspiro[4.4]non-3-en-1-yl)acetamide
N-(3-chlorophenyl)-2-(2-oxo-3-phenyl-1,4-diazaspiro[4.4]non-3-en-1-yl)acetamide
N-(4-chlorophenyl)-2-(2-oxo-3-phenyl-1,4-diazaspiro[4.4]non-3-en-1-yl)acetamide
N-(2-fluorophenyl)-2-[3-(4-fluorophenyl)-2-oxo-1,4-diazaspiro[4.4]non-3-en-1-yl]acetamide
2-[3-(4-fluorophenyl)-2-oxo-1,4-diazaspiro[4.4]non-3-en-1-yl]-N-phenylacetamide
N-(3-methylphenyl)-2-(2-oxo-3-phenyl-1,4-diazaspiro[4.4]non-3-en-1-yl)acetamide
2-[3-(4-fluorophenyl)-2-oxo-1,4-diazaspiro[4.4]non-3-en-1-yl]-N-(3-methylphenyl)acetamide
N-(2,3-dimethylphenyl)-2-[3-(4-fluorophenyl)-2-oxo-1,4-diazaspiro[4.4]non-3-en-1-yl]acetamide
2-[3-(4-fluorophenyl)-2-oxo-1,4-diazaspiro[4.4]non-3-en-1-yl]-N-[4-(methyloxy)phenyl]acetamide
N-(2,4-dimethylphenyl)-2-[3-(4-fluorophenyl)-2-oxo-1,4-diazaspiro[4.4]non-3-en-1-yl]acetamide
2-[3-(4-fluorophenyl)-2-oxo-1,4-diazaspiro[4.4]non-3-en-1-yl]-N-(4-methylphenyl)acetamide
N-(3-fluorophenyl)-2-[3-(4-fluorophenyl)-2-oxo-1,4-diazaspiro[4.4]non-3-en-1-yl]acetamide
N-(3-chloro-4-fluorophenyl)-2-[3-(4-fluorophenyl)-2-oxo-1,4-diazaspiro[4.4]non-3-en-1-yl]acetamide
N-(4-chlorophenyl)-2-[3-(4-fluorophenyl)-2-oxo-1,4-diazaspiro[4.4]non-3-en-1-yl]acetamide
N-(3,4-dimethylphenyl)-2-[3-(4-fluorophenyl)-2-oxo-1,4-diazaspiro[4.4]non-3-en-1-yl]acetamide
N-(4-fluorophenyl)-2-[3-(4-fluorophenyl)-2-oxo-1,4-diazaspiro[4.4]non-3-en-1-yl]acetamide
N-[3-(methyloxy)phenyl]-2-(2-oxo-3-phenyl-1,4-diazaspiro[4.4]non-3-en-1-yl)acetamide
N-(4-Fluoro-3-methylphenyl)-2-{3-[4-(methyloxy)phenyl]-2-oxo-1,4-diazaspiro[4.5]dec-3-en-1-yl}acetamide
N-[2-(Methyloxy)phenyl]-2-[3-(4-methylphenyl)-2-oxo-1,4-diazaspiro[4.5]dec-3-en-1-yl]acetamide
2-[3-(4-Chlorophenyl)-2-oxo-1,4-diazaspiro[4.5]dec-3-en-1-yl]-N-(2,3-difluorophenyl)acetamide
2-[3-(4-chlorophenyl)-2-oxo-1,4-diazaspiro[4.5]dec-3-en-1-yl]-N-(2-methylphenyl)acetamide
N-(2,4-dimethylphenyl)-2-[3-(4-methylphenyl)-2-oxo-1,4-diazaspiro[4.5]dec-3-en-1-yl]acetamide
N-(3,4-difluorophenyl)-2-[3-(4-methylphenyl)-2-oxo-1,4-diazaspiro[4.5]dec-3-en-1-yl]acetamide
N-(3-fluorophenyl)-2-[3-(4-methylphenyl)-2-oxo-1,4-diazaspiro[4.5]dec-3-en-1-yl]acetamide
N-(2-methylphenyl)-2-[3-(4-methylphenyl)-2-oxo-1,4-diazaspiro[4.5]dec-3-en-1-yl]acetamide
2-[3-(4-chlorophenyl)-2-oxo-1,4-diazaspiro[4.5]dec-3-en-1-yl]-N-(2,3-dimethylphenyl)acetamide
2-[3-(4-chlorophenyl)-2-oxo-1,4-diazaspiro[4.5]dec-3-en-1-yl]-N-phenylacetamide
N-(3-fluorophenyl)-2-{3-[4-(methyloxy)phenyl]-2-oxo-1,4-diazaspiro[4.5]dec-3-en-1-yl}acetamide
N-(2,4-dimethylphenyl)-2-{3-[4-(methyloxy)phenyl]-2-oxo-1,4-diazaspiro[4.5]dec-3-en-1-yl}acetamide
N-(3-chlorophenyl)-2-[3-(4-chlorophenyl)-2-oxo-1,4-diazaspiro[4.5]dec-3-en-1-yl]acetamide
N-(4-chloro-3-methylphenyl)-2-{3-[4-(methyloxy)phenyl]-2-oxo-1,4-diazaspiro[4.5]dec-3-en-1-yl}acetamide
2-{3-[4-(methyloxy)phenyl]-2-oxo-1,4-diazaspiro[4.5]dec-3-en-1-yl}-N-(2,3,4-trifluorophenyl)acetamide
and solvates thereof.

The compounds of formula (I) may have the ability to crystallise in more than one form. This is a characteristic known as polymorphism, and it is understood that such polymorphic forms (“polymorphs”) are within the scope of formula (I). Polymorphism generally can occur as a response to changes in temperature or pressure or both and can also result from variations in the crystallisation process. Polymorphs can be distinguished by various physical characteristics known in the art such as x-ray diffraction patterns, solubility, and melting point.

Certain of the compounds described herein may exist in stereoisomeric forms (i.e. they may contain one or more asymmetric carbon atoms or may exhibit cis-trans isomerism). The individual stereoisomers (enantiomers and diastereoisomers) and mixtures of these are included within the scope of the present invention. Likewise, it is understood that compounds of formula (I) may exist in tautomeric forms other than that shown in the formula and these are also included within the scope of the present invention.

As referred to above, individual enantiomers of compounds of formula (I) may be prepared and an indication of the preferred stereochemistry for such enantiomers has been given. In a preferred embodiment, an optically pure enantiomer is desired. The term “optically pure enantiomer” means that the compound contains greater than about 90% of the desired isomer by weight, preferably greater than about 95% of the desired isomer by weight, and most preferably greater than about 99% of the desired isomer by weight, said weight percent based upon the total weight of the isomer(s) of the compound.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the working Examples.

Compounds of general formula (I) may be prepared by methods known in the art of organic synthesis as set forth in part by the following synthesis schemes. It is also recognised that in all of the schemes described below, it is well understood that protecting groups for sensitive or reactive groups are employed where necessary in accordance with general principles of chemistry. Protecting groups are manipulated according to standard methods of organic synthesis (T. W. Greene and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula (I). Those skilled in the art will recognise if a stereocentre exists in compounds of formula (I). Accordingly, the present invention includes both possible stereoisomers and includes not only racemic compounds but the individual enantiomers as well. Where the stereochemistry is indicated as being variable at certain positions, a mixture of stereoisomers may be obtained, this mixture having been separated where indicated. Stereoisomers may be separated by high-performance liquid chromatography or other appropriate means. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

Typical reaction routes for the preparation of a compound of formula (I) as hereinbefore defined, are shown below:

Compounds of formula (I) can be prepared by reacting a compound of formula (II) with a base, for example sodium hydride, in a suitable inert solvent, for example dimethylformamide, followed by treatment with a compound of formula (III) as shown in Scheme 1.

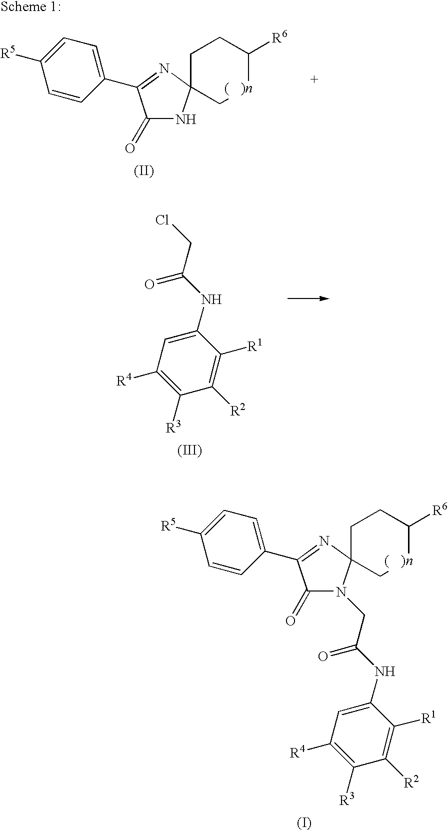

Compounds of formula (III) can be prepared by standard methods, for example as shown in Scheme 2. For example an aniline of formula (IV) may be combined with chloroacetyl chloride in an inert solvent, for example dioxan, and heated to give a compound of formula (III).

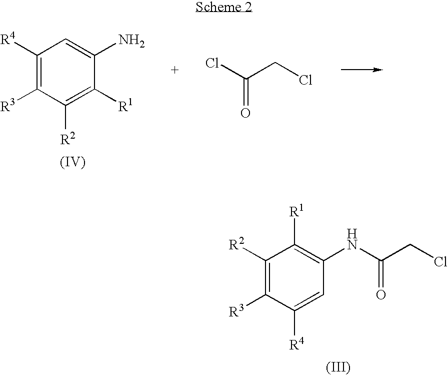

Compounds of formula (II) may be prepared by desulphurisation of compounds of formula (V) using an oxidising agent, for example hydrogen peroxide as shown for example in Scheme 3.

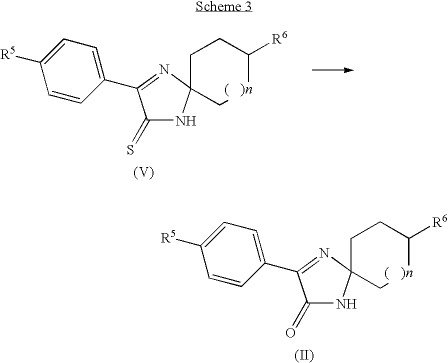

Compounds of formula (V) can be prepared by treating a ketothioamide of formula (VI) with the appropriate ketone of formula (VII) in the presence of a source of ammonia, for example ammonium acetate as shown in Scheme 4. Preferably this reaction is performed in a solvent, for example isopropanol, at room or elevated temperature, preferably elevated temperature, for example at reflux.

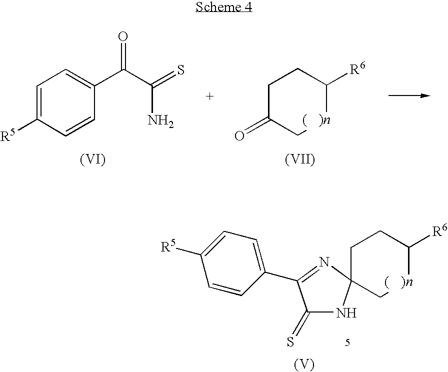

Thioamides of formula (VI) can be prepared from acylnitriles of formula (VIII) by treating with, for example hydrogen sulphide in the presence of an organic base, for example triethylamine in an inert solvent, for example diethyl ether at room temperature. Acylnitriles of formula (VIII) can be prepared from the appropriate acid chloride (IX) and a source of cyanide, conveniently copper (I) cyanide, at elevated temperatures, for example greater than 150° C. preferably in the absence of solvent.

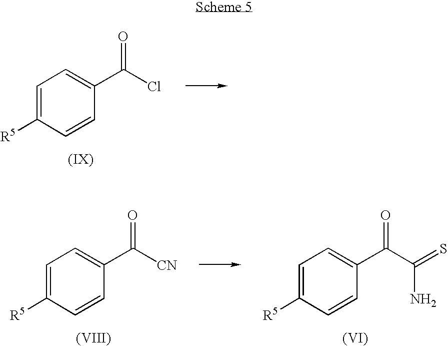

Alternatively, compounds of formula (II) can be synthesised as shown in Scheme 6.

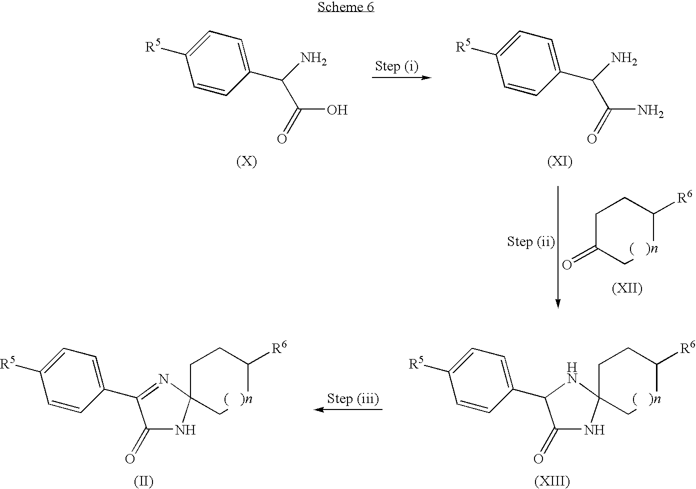

wherein R⁵, R⁶ and n are as defined for formula (I).

The arylglycine of formula (X) can be converted, step (i), to the corresponding arylglycinamide of formula (XI) by standard methods, for example, by reaction of compounds of formula (X) with thionyl chloride or acetyl chloride in methanol, followed by subsequent reaction of the intermediate methyl ester hydrochloride with aqueous ammonia.

Arylglycinamides of formula (XI) can be converted to compounds of formula (XIII), step (ii), by condensation with ketones of formula (XII), for example, by heating in an inert solvent such as methanol, in the presence or absence of a catalyst such as H—Y zeolites.

Oxidation of compounds of formula (XIII), step (iii), to afford compounds of formula (II) can be achieved by methods known in the art, for example, by reaction with N-bromosuccinimide in an inert solvent, such as dichloromethane.

Compounds of formula (II) can also be converted to compounds of formula (I) as shown in Scheme 7.

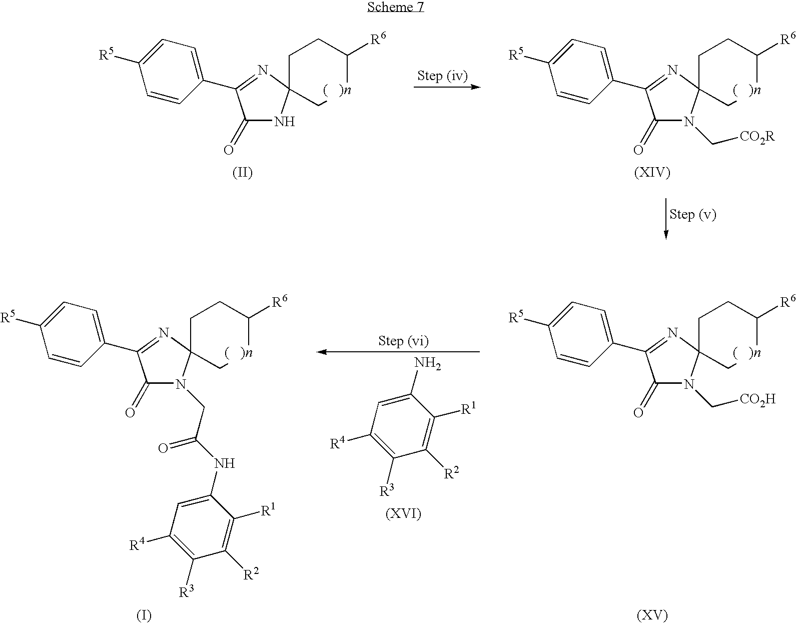

wherein R¹, R², R³, R⁴, R⁵, R⁶ and n are as defined for compounds of formula (I).

Compounds of formula (XIV) can be prepared using standard methods from compounds of formula (II), step (iv), for example, by reaction with an appropriate haloester in the presence of a base, such as sodium hydride or potassium carbonate, in a suitable inert solvent, such as dimethylformamide, at room temperature or elevated temperature as appropriate.

Removal of the ester group R from compounds of formula (XIV) to afford the acids of formula (XV), step (v), can be achieved by known methods, for example by use of a base, such as sodium hydroxide, in an inert solvent, such as aqueous methanol or aqueous ethanol, with or without heating as appropriate.

Compounds of formula (XV) can be converted to compounds of formula (I), step (vi), by reaction with an aniline of formula (XVI) using a variety of methods known in the art. For example, the acylation step (vi) can be achieved by reaction of the acid (XV) with an aniline of formula (XVI), in an inert solvent, such as dichloromethane in the presence of a coupling reagent, for example a diimide reagent such as N,N dicyclohexylcarbodiimide (DCC), N-(3-(dimethylamino)propyl)-N-ethylcarbodiimide hydrochloride (EDC), or O-(7-azabenzotriazol-1-yl)-1,1,3,3-tetramethyluronium hexafluoro phosphate (HATU). Alternatively, compounds of formula (XV) are converted to compounds of formula (XVII)

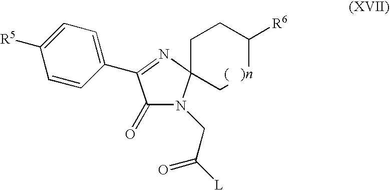

wherein R⁵, R⁶ and n are as defined in formula (I) and L represents a suitable leaving group. Examples of leaving groups include halogen, OC(═O)alkyl, OC(═O)O-alkyl and OSO₂Me. L may be halogen and acylation in step (vi) may be carried out in an inert solvent such as dichloromethane, in the presence of a base, such as triethylamine.

Within the scheme there is scope to convert a group R¹ into another group R¹ and similarly for groups R², R³, R⁴, R⁵ and R⁶.

Compounds of formula (I) can be converted into further compounds of formula (I) using standard techniques.

In one aspect, the present invention provides a method of preparing a compound of formula (I) or a solvate thereof, the method comprising

(a) reacting a compound of formula (II):

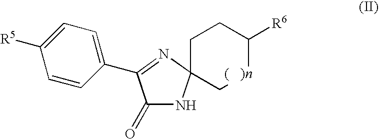

wherein R⁵, R⁶ and n are as defined for formula (I), with a compound of formula (III):

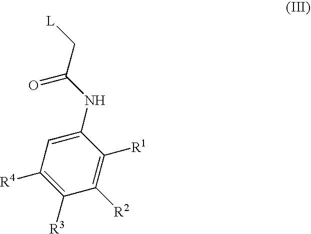

wherein R¹, R², R³ and R⁴ are as defined for formula (I), and L is a leaving group; or

(b) reacting a compound of formula (XV):

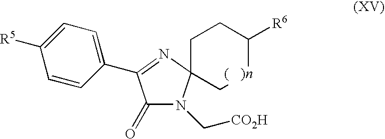

wherein R⁵, R⁶ and n are as defined for formula (I), with a compound of formula (XVI):

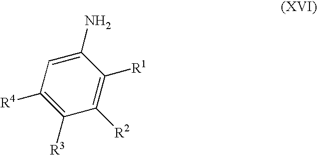

wherein R¹, R², R³ and R⁴ are as defined for formula (I); and thereafter optionally:
form a solvate and/or
convert a compound of formula (I) to another compound of formula (I).

The compounds of the present invention inhibit the GlyT1 transporter. The compounds may selectively inhibit the GlyT1 transporter over the GlyT2 transporter. Some compounds of the invention may have mixed GlyT-1/GlyT-2 activity.

Such compounds would be suitable for the treatment of certain neurological and neuropsychiatric disorders. As used herein, the terms “treatment” and “treating” refer to the alleviation and/or cure of established symptoms as well as prophylaxis.

The affinities of the compounds of this invention for the GlyT1 transporter can be determined by the following assay.

HEK293 cells expressing the Glycine (Type 1) transporter were grown in cell culture medium [DMEM/NUT mix F12 containing 2 mM L-Glutamine, 0.8 mg/mL G418 and 10% heat inactivated fetal calf serum] at 37° C. and 5% CO₂. Cells grown to 70-80% confluency in T175 flasks were harvested and resuspended at 4×10⁵ cells/mL in assay buffer [140 mM NaCl, 5.4 mM KCl, 1.8 mM CaCl₂, 0.8 mM MgSO₄, 20 mM HEPES, 5 mM glucose and 5 mM alanine, pH 7.4]. Compounds were serially diluted 2.5-fold in DMSO from a top concentration of 2.5 mM with each compound giving a 11 data point dose-response. 100 nL of compound at each concentration was added to the assay plate. An equal volume of Leadseeker™ WGA SPA beads (12.5 mg/ml suspended in assay buffer) was added to the cell suspension and 5 μL of the cell/bead suspension transferred to each well of a 384-well white solid bottom plate (1,000 cells/well) containing 100 nL of test compounds. Substrate (5 μL) was added to each well [1:100 dilution of [³H]-glycine stock in assay buffer containing 2.5 μM glycine). Final DMSO concentration was 1% v/v. Data was collected using a Perkin Elmer Viewlux. pIC₅₀ values were determined using ActivityBase.

Compounds are considered to have activity at the GlyT1 transporter if they have a pIC₅₀ of 5.0 or above. The example compounds below and the individually named compounds above were found to have a pIC₅₀ at the GlyT1 transporter of greater than 5.0. Some compounds of the invention were found to have a pIC₅₀ at the GlyT1 transporter of greater than 7.0.

Accordingly, in a further aspect of the invention, there is provided a compound of formula (I) as hereinbefore described or a solvate thereof, for use in the treatment of a disorder mediated by GlyT1.

As used herein, the term “a disorder mediated by GlyT1” refers to a disorder that may be treated by the administration of a medicament that alters the activity of the GlyT1 transporter. As hereinbefore described, the action of GlyT1 transporters affects the local concentration of glycine around NMDA receptors. As a certain amount of glycine is needed for the efficient functioning of NMDA receptors, any change to that local concentration can affect NMDA-mediated neurotransmission. As hereinbefore described, changes in NMDA-mediated neurotransmission have been implicated in certain neuropsychiatric disorders such as dementia, depression and psychoses, for example schizophrenia, and learning and memory disorders, for example attention deficit disorders and autism. Thus, alterations in the activity of the GlyT1 transporter are expected to influence such disorders.

The disorders mediated by GlyT1 referred to herein include neurological and neuropsychiatric disorders, including psychoses such as schizophrenia, dementia and other forms of impaired cognition such as attention deficit disorders and organic brain syndromes. Other neuropsychiatric disorders include drug-induced (phencyclidine, ketamine and other dissociative anesthetics, amphetamine and other psychostimulants and cocaine) psychosis, psychosis associated with affective disorders, brief reactive psychosis, schizoaffective psychosis, and psychosis NOS, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), and NMDA receptor-related disorders such as autism, depression, benign forgetfulness, childhood learning disorders and closed head injury.

In another aspect of the invention, there is provided a method of treating a mammal, including a human, suffering from or susceptible to a disorder mediated by GlyT1, which comprises administering an effective amount of a compound of formula (I) as hereinbefore defined or a solvate thereof.

In another aspect of the invention, there is provided use of a compound of formula (I) as hereinbefore defined or a solvate thereof in the preparation of a medicament for the treatment of a disorder mediated by GlyT1.

Preferably, the disorder mediated by GlyT1 to be treated by the use or method as hereinbefore described is a psychosis, including schizophrenia, dementia and attention deficit disorders, particularly schizophrenia.

As used herein, the term “effective amount” means that amount of a drug or pharmaceutical agent that will elicit the biological or medical response of a tissue, system, animal or human that is being sought, for instance, by a researcher or clinician.

Compounds for use according to the invention may be administered as the raw material but the active ingredients are preferably provided in the form of pharmaceutical compositions.

Accordingly, in a further aspect of the invention, there is provided a pharmaceutical composition comprising a compound of formula (I) as hereinbefore described or a solvate thereof, and at least one pharmaceutically acceptable carrier, diluent or excipient.

These pharmaceutical compositions may be used in the treatment of clinical conditions for which a GlyT1 inhibitor is indicated such as, for example, schizophrenia. The carrier must be pharmaceutically acceptable to the recipient and must be compatible with, i.e. not have a deleterious effect upon, the other ingredients in the composition. The carrier may be a solid or a liquid and is preferably formulated with at least one compound of formula (I) or a solvate thereof as a unit dose formulation. If desired, other physiologically active ingredients may also be incorporated in the pharmaceutical compositions of the invention.

Within the context of the present invention, the terms used herein are classified in the Diagnostic and Statistical Manual of Mental Disorders, 4^{th} Edition, published by the American Psychiatric Association (DSM-IV) and/or the International Classification of Diseases, 10^{th} Edition (ICD-10). The various subtypes of the disorders mentioned herein are contemplated as part of the present invention. Numbers in brackets after the listed diseases below refer to the classification code in DSM-IV.

In particular, the compounds of formula (I) are of use in the treatment of schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-Induced Psychotic Disorder including the subtypes With Delusions (293.81) and With Hallucinations (293.82); and Psychotic Disorder Not Otherwise Specified (298.9).

The compounds of formula (I) are also of use in the treatment of mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90).

The compounds of formula (I) are also of use in the treatment of anxiety disorders including Panic Attack, Agoraphobia, Panic Disorder, Agoraphobia Without History of Panic Disorder (300.22), Specific Phobia (300.29) including the subtypes Animal Type, Natural Environment Type, Blood-Injection-Injury Type, Situational Type and Other Type), Social Phobia (300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder and Anxiety Disorder Not Otherwise Specified (300.00).

The compounds of formula (I) are also of use in the treatment of substance-related disorders including Substance Use Disorders such as Substance Dependence and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

The compounds of formula (I) are also of use in the treatment of sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition; and Substance-Induced Sleep Disorder including the subtypes Insomnia Type, Hypersomnia Type, Parasomnia Type and Mixed Type.

The compounds of formula (I) are also of use in the treatment of eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; and Eating Disorder Not Otherwise Specified (307.50).

The compounds of formula (I) are also of use in the treatment of Autistic Disorder (299.00); Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23).

The compounds of formula (I) are also of use in the treatment of Personality Disorders including the subtypes Paranoid Personality Disorder (301.0), Schizoid Personality Disorder (301.20), Schizotypal Personality Disorder (301,22), Antisocial Personality Disorder (301.7), Borderline Personality Disorder (301,83), Histrionic Personality Disorder (301.50), Narcissistic Personality Disorder (301,81), Avoidant Personality Disorder (301.82), Dependent Personality Disorder (301.6), Obsessive-Compulsive Personality Disorder (301.4) and Personality Disorder Not Otherwise Specified (301.9).

The compounds of Formula (I) are also of use in the enhancement of cognition including the treatment of cognition impairment in other diseases such as schizophrenia, bipolar disorder, depression, other psychiatric disorders and psychotic conditions associated with cognitive impairment. Within the context of the present invention, the term cognitive impairment includes for example the treatment of impairment of cognitive functions including attention, orientation, learning disorders, memory (i.e. memory disorders, amnesia, amnesic disorders, transient global amnesia syndrome and age-associated memory impairment) and language function; cognitive impairment as a result of stroke, Alzheimer's disease, Huntington's disease, Pick disease, Aids-related dementia or other dementia states such as Multiinfarct dementia, alcoholic dementia, hypotiroidism-related dementia, and dementia associated to other degenerative disorders such as cerebellar atrophy and amyotropic lateral sclerosis; other acute or sub-acute conditions that may cause cognitive decline such as delirium or depression (pseudodementia states) trauma, head trauma, age related cognitive decline, stroke, neurodegeneration, drug-induced states, neurotoxic agents, mild cognitive impairment, age related cognitive impairment, autism related cognitive impairment, Down's syndrome, cognitive deficit related to psychosis, and post-electroconvulsive treatment related cognitive disorders; and dyskinetic disorders such as Parkinson's disease, neuroleptic-induced parkinsonism, and tardive dyskinesias.

The compounds of formula (I) are also of use in the treatment of sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

The invention also provides a compound of formula (I) as hereinbefore described or a solvate thereof for use in the treatment of schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, Parkinson's disease, dyskinetic disorders, depression, bipolar disorder, cognitive impairment, obesity, emesis, movement disorders, obsessive-compulsive disorders, amnesia, aggression, vertigo, dementia and circadian rhythm disorders.

The invention also provides a compound of formula (I) as hereinbefore described or a solvate thereof for use in the treatment of psychotic disorders, schizophrenia, Parkinson's disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.

In another aspect of the invention, there is provided a method of treating a mammal, including a human, suffering from or susceptible to a disorder mediated by GlyT1, which comprises administering an effective amount of a compound of formula (I) as hereinbefore defined or a solvate thereof.

The invention also provides a method of treating schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, Parkinson's disease, dyskinetic disorders, depression, bipolar disorder, cognitive impairment, obesity, emesis, movement disorders, obsessive-compulsive disorders, amnesia, aggression, vertigo, dementia and circadian rhythm disorders which comprises administering to a mammal in need thereof an effective amount of a compound of formula (I) as hereinbefore described or a solvate thereof.

The invention also provides a method of treating psychotic disorders, schizophrenia, Parkinson's disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders which comprises administering to a mammal in need thereof an effective amount of a compound of formula (I) as hereinbefore described or a solvate thereof.

The compounds of formula (I) are also of use as anticonvulsants. The compounds of formula (I) are thus useful in the treatment of convulsions in mammals, and particularly epilepsy in humans. “Epilepsy” is intended to include the following seizures: simple partial seizures, complex partial seizures, secondary generalised seizures, generalised seizures including absence seizures, myoclonic seizures, clonic seizures, tonic seizures, tonic clonic seizures and atonic seizures. The invention also provides a method of treating convulsions, which comprises administering to a mammal in need thereof an effective amount of a compound of formula (I) as hereinbefore described or a solvate thereof. Treatment of epilepsy may be carried out by the administration of a non-toxic anticonvulsant effective amount of a compound of the formula (I) or a solvate thereof, or a composition as hereinbefore defined.

The compounds of formula (I) also find use in the treatment of neuropathic pain, for example in diabetic neuropathy, sciatica, non-specific lower back pain, multiple sclerosis pain, fibromyalgia, HIV-related neuropathy, neuralgia such as post-herpetic neuralgia and trigeminal neuralgia and pain resulting from physical trauma, amputation, cancer, toxins or chronic inflammatory conditions.

In another aspect of the invention, there is provided use of a compound of formula (I) as hereinbefore defined or a solvate thereof in the preparation of a medicament for the treatment of a disorder mediated by GlyT1.

Preferably, the disorder mediated by GlyT1 to be treated by the use or method as hereinbefore described is a psychosis, including schizophrenia, dementia and attention deficit disorders, particularly schizophrenia.

The invention also provides the use of a compound of formula (I) as hereinbefore described or a solvate thereof in the manufacture of a medicament for the treatment of schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, Parkinson's disease, dyskinetic disorders, depression, bipolar disorder, cognitive impairment, obesity, emesis, movement disorders, obsessive-compulsive disorders, amnesia, aggression, vertigo, dementia and circadian rhythm disorders.

The invention also provides the use of a compound of formula (I) as hereinbefore described or a solvate thereof in the manufacture of a medicament for the treatment of psychotic disorders, schizophrenia, Parkinson's disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.

As used herein, the term “effective amount” means that amount of a drug or pharmaceutical agent that will elicit the biological or medical response of a tissue, system, animal or human that is being sought, for instance, by a researcher or clinician.

Compounds for use according to the invention may be administered as the raw material but the active ingredients are preferably provided in the form of pharmaceutical compositions.

Accordingly, in a further aspect of the invention, there is provided a pharmaceutical composition comprising a compound of formula (I) as hereinbefore described or a solvate thereof, and at least one pharmaceutically acceptable carrier, diluent or excipient.

These pharmaceutical compositions may be used in the treatment of clinical conditions for which a GlyT1 inhibitor is indicated such as, for example, schizophrenia. The carrier must be pharmaceutically acceptable to the recipient and must be compatible with, i.e. not have a deleterious effect upon, the other ingredients in the composition. The carrier may be a solid or a liquid and is preferably formulated with at least one compound of formula (I) or a solvate thereof as a unit dose formulation. If desired, other physiologically active ingredients may also be incorporated in the pharmaceutical compositions of the invention.

It will be appreciated by those skilled in the art that the compounds according to the invention may advantageously be used in conjunction with one or more other therapeutic agents, for instance, different antidepressant agents such as 5HT3 antagonists, serotonin agonists, NK-1 antagonists, selective serotonin reuptake inhibitors (SSRI), noradrenaline re-uptake inhibitors (SNRI), tricyclic antidepressants, dopaminergic antidepressants, H3 antagonists, 5HT1A antagonists, 5HT1B antagonists, 5HT1D antagonists, D1 agonists, M1 agonists and/or anticonvulsant agents, as well as atypical antipsychotic drugs and cognitive enhancers.

Suitable 5HT3 antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

Suitable anticonvulsant agents which may be used in combination of the compounds of the invention include for example divalproex, carbamazepine and diazepam.

Suitable atypical antipsychotic drugs which may be used in combination of the compounds of the invention include for example risperidone, olanzapine, ziprasidone, aripiprazole and clozapine.

It will be appreciated that the compounds of the combination or composition may be administered simultaneously (either in the same or different pharmaceutical formulations), separately or sequentially.

The compounds of formula (I) and their solvates thereof are also suitable for combination with other typical and atypical antipsychotics to provide improved treatment of psychotic disorders. Particular advantages associated with the combinations, uses and methods of treatment of compounds of formula (I) and their solvates thereof include equivalent or improved efficacy at doses of administration which are lower than those commonly used for the individual components. Improved treatments of positive symptoms and/or negative symptoms and/or cognitive symptoms of the psychotic disorder may also be observed. The combinations, uses and methods of treatment of the invention may also provide advantages in treatment of patients who fail to respond adequately or who are resistant to treatment with certain neuroleptic agents.

The combination therapies of the invention are preferably administered adjunctively. By adjunctive administration is meant the coterminous or overlapping administration of each of the components in the form of separate pharmaceutical compositions or devices. This regime of therapeutic administration of two or more therapeutic agents is referred to generally by those skilled in the art and herein as adjunctive therapeutic administration; it is also known as add-on therapeutic administration. Any and all treatment regimes in which a patient receives separate but coterminous or overlapping therapeutic administration of the compounds of formula (I) or a solvate thereof and at least one neuroleptic agent are within the scope of the current invention. In one embodiment of adjunctive therapeutic administration as described herein, a patient is typically stabilised on a therapeutic administration of one or more of the of the components for a period of time and then receives administration of another component. Within the scope of this invention, it is preferred that the compounds of formula (I) or a solvate thereof is administered as adjunctive therapeutic treatment to patients who are receiving administration of at least one neuroleptic agent, but the scope of the invention also includes the adjunctive therapeutic administration of at least one neuroleptic agent to patients who are receiving administration of compounds of formula (I) or a solvate thereof.

The combination therapies of the invention may also be administered simultaneously. By simultaneous administration is meant a treatment regime wherein the individual components are administered together, either in the form of a single pharmaceutical composition or device comprising or containing both components, or as separate compositions or devices, each comprising one of the components, administered simultaneously. Such combinations of the separate individual components for simultaneous combination may be provided in the form of a kit-of-parts.

In a further aspect therefore, the invention provides a method of treatment of a psychotic disorder by adjunctive therapeutic administration of compounds of formula (I) or a solvate thereof to a patient receiving therapeutic administration of at least one neuroleptic agent. In a further aspect, the invention provides the use of compounds of formula (I) or a solvate thereof in the manufacture of a medicament for adjunctive therapeutic administration for the treatment of a psychotic disorder in a patient receiving therapeutic administration of at least one neuroleptic agent. The invention further provides compounds of formula (I) or a solvate thereof for use for adjunctive therapeutic administration for the treatment of a psychotic disorder in a patient receiving therapeutic administration of at least one neuroleptic agent.

In a further aspect, the invention provides a method of treatment of a psychotic disorder by adjunctive therapeutic administration of at least one neuroleptic agent to a patient receiving therapeutic administration of compounds of formula (I) or a solvate thereof. In a further aspect, the invention provides the use of at least one neuroleptic agent in the manufacture of a medicament for adjunctive therapeutic administration for the treatment of a psychotic disorder in a patient receiving therapeutic administration of compounds of formula (I) or a solvate thereof. The invention further provides at least one neuroleptic agent for adjunctive therapeutic administration for the treatment of a psychotic disorder in a patient receiving therapeutic administration of compounds of formula (I) or a solvate thereof.

In a further aspect, the invention provides a method of treatment of a psychotic disorder by simultaneous therapeutic administration of compounds of formula (I) or a solvate thereof in combination with at least one neuroleptic agent. The invention further provides the use of a combination of compounds of formula (I) or a solvate thereof and at least one neuroleptic agent in the manufacture of a medicament for simultaneous therapeutic administration in the treatment of a psychotic disorder. The invention further provides the use of compounds of formula (I) or a solvate thereof in the manufacture of a medicament for simultaneous therapeutic administration with at least one neuroleptic agent in the treatment of a psychotic disorder. The invention further provides compounds of formula (I) or a solvate thereof for use for simultaneous therapeutic administration with at least one neuroleptic agent in the treatment of a psychotic disorder. The invention further provides the use of at least one neuroleptic agent in the manufacture of a medicament for simultaneous therapeutic administration with compounds of formula (I) or a solvate thereof in the treatment of a psychotic disorder.

In further aspects, the invention provides a method of treatment of a psychotic disorder by simultaneous therapeutic administration of a pharmaceutical composition comprising compounds of formula (I) or a solvate thereof and at least one mood stabilising or antimanic agent, a pharmaceutical composition comprising compounds of formula (I) or a solvate thereof and at least one mood stabilising or antimanic agent, the use of a pharmaceutical composition comprising compounds of formula (I) or a solvate thereof and at least one mood stabilising or antimanic agent in the manufacture of a medicament for the treatment of a psychotic disorder, and a pharmaceutical composition comprising compounds of formula (I) or a solvate thereof and at least one mood stabilising or antimanic agent for use in the treatment of a psychotic disorder.

In a further aspect, the invention provides a kit-of-parts for use in the treatment of a psychotic disorder comprising a first dosage form comprising compounds of formula (I) or a solvate thereof and one or more further dosage forms each comprising a neuroleptic agent for simultaneous therapeutic administration.

Within the context of the present invention, the term psychotic disorder includes those disorders mentioned above, such as schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, dyskinetic disorders, depression, bipolar disorder, cognitive impairment and obsessive-compulsive disorders and all the various forms of the disorders as mentioned herein which are contemplated as part of the present invention.

Examples of neuroleptic/antipsychotic drugs that are useful in the present invention include, but are not limited to: butyrophenones, such as haloperidol, pimozide, and droperidol; phenothiazines, such as chlorpromazine, thioridazine, mesoridazine, trifluoperazine, perphenazine, fluphenazine, thiflupromazine, prochlorperazine, and acetophenazine; thioxanthenes, such as thiothixene and chlorprothixene; thienobenzodiazepines; dibenzodiazepines; benzisoxazoles; dibenzothiazepines; imidazolidinones; benzisothiazolyl-piperazines; triazine such as lamotrigine; dibenzoxazepines, such as loxapine; dihydroindolones, such as molindone; aripiprazole; and derivatives thereof that have antipsychotic activity.

Examples of neuroleptic drugs that are preferred for use in the present invention are shown in Table 1.

TABLE 1
Neuroleptic drugs

| | | | | Dosage |
|---|---|---|---|---|
| Common | | Route of | | Range and |
| Name | Trade Name | Administration | Form | (Median)^{a} |
| Clozapine | CLOZARIL | oral | tablets | 12.5-900 mg/day |
| | | | | (300-900 mg/day) |
| Olanzapine | ZYPREXA | oral | tablets | 5-25 mg/day |
| | | | | (10-25 mg/day) |
| Ziprasidone | GEODON | oral | capsules | 20-80 mg/twice a day |
| | | | | (80-160 mg/day) |
| Risperidone | RISPERDAL | oral | solution tablets | 2-16 mg/day |
| | | | | tablets |
| | | | | (4-12 mg/day) |
| Quetiapine | SEROQUEL | oral | tablets | 50-900 mg/day |
| fumarate | | | | (300-900 mg/day) |
| Sertindole | SERLECT | | | (4-24 mg/day) |
| Amisulpride | | | | |
| Haloperidol | HALDOL | oral | tablets | 1-100 mg/day |
| | | | | (1-15 mg/day) |
| Haloperidol | HALDOL | parenteral | injection | |
| Decanoate | Decanoate | | | |
| Haloperidol | HALDOL | oral | solution | |
| lactate | INTENSOL | parenteral | injection | |
| Chlorpromazine | THORAZINE | rectal | suppositories | 30-800 mg/day |
| | | oral | capsules | (200-500 mg/day) |
| | | | solution | |
| | | | tablets | |
| | | parenteral | injection | |
| Fluphenazine | PROLIXIN | | | 0.5-40 mg/day |
| | | | | (1-5 mg/day) |
| Fluphenazine | PROLIXIN | parenteral | injection | (about one-half |
| decanoate | Decanoate | | | the dosage |
| | | | | shown for oral) |
| Fluphenazine | PROLIXIN | parenteral | injection | (same as above |
| enanthate | | | | |
| Fluphenazine | PROLIXIN | oral | elixer | |
| hydrochloride | | | solution | |
| | | parenteral | injection | |
| Thiothixene | NAVANE | oral | capsules | 6-60 mg/day |
| | | | | (8-30 mg/day) |
| Thiothixene | NAVANE | oral | solution | |
| hydrochloride | | parenteral | injection | |
| Trifluoperazine | STELAZINE | | | (2-40 mg/day) |
| Perphenazine | TRILAFON | oral | solution | 12-64 mg/day |
| | | | tablets | (16-64 mg/day) |
| | | parenteral | injection | |
| Perpehazine | ETRAFON | oral | tablets | |
| and | TRIAVIL | | | |
| Amitriptyline | | | | |
| hydrochloride | | | | |
| Thioridazine | MELLARIL | oral | suspension | 150-800 mg/day |
| | | | solution | |
| | | | tablets | (100-300 mg/day) |
| Mesoridazine | | | | (30-400 mg/day) |
| Molindone | MOBAN | | | 50-225 mg/day |
| | | | | (15-150 mg/day) |
| Molindone | MOBAN | oral | solution | |
| hydrochloride | | | | |
| Loxapine | LOXITANE | | | 20-250 mg/day |
| | | | | (60-100 mg/dav) |
| Loxapine | LOXITANE | oral | solution | |
| hydrochloride | | parenteral | injection | |
| Loxapine | LOXITANE | oral | capsules | |
| succinate | | | | |
| Pimozide | | | | (1-10 mg/day) |
| Flupenthixol | | | | |
| Promazine | SPARINE | | | |
| Triflupromazine | VESPRIN | | | |
| Chlorprothixene | TARACTAN | | | |
| Droperidol | INAPSINE | | | |
| Acetophenazine | TINDAL | | | |
| Prochlorperazine | COMPAZINE | | | |
| Methotrimeprazine | NOZINAN | | | |
| Pipotiazine | PIPOTRIL | | | |
| Aripiprazole | | | | |
| Hoperidone | | | | |

Examples of tradenames and suppliers of selected neuroleptic drugs are as follows: clozapine (available under the tradename CLOZARIL®, from Mylan, Zenith Goldline, UDL, Novartis); olanzapine (available under the tradename ZYPREX®, from Lilly; ziprasidone (available under the tradename GEODON®, from Pfizer); risperidone (available under the tradename RISPERDAL®, from Janssen); quetiapine fumarate (available under the tradename SEROQUEL®, from AstraZeneca); haloperidol (available under the tradename HALDOL®, from Ortho-McNeil); chlorpromazine (available under the tradename THORAZINE®, from SmithKline Beecham (GSK); fluphenazine (available under the tradename PROLIXIN®, from Apothecon, Copley, Schering, Teva, and American Pharmaceutical Partners, Pasadena); thiothixene (available under the tradename NAVANE®, from Pfizer); trifluoperazine (10-[3-(4-methyl-1-piperazinyl)propyl]-2-(trifluoromethyl)phenothiazine dihydrochloride, available under the tradename STELAZINE®, from Smith Klein Beckman; perphenazine (available under the tradename TRILAFON®; from Schering); thioridazine (available under the tradename MELLARIL®; from Novartis, Roxane, HiTech, Teva, and Alpharma); molindone (available under the tradename MOBAN®, from Endo); and loxapine (available under the tradename LOXITANE®; from Watson). Furthermore, benperidol (Glianimon®), perazine (Taxilan®) or melperone (Eunerpan®)) may be used.

Other preferred neuroleptic drugs include promazine (available under the tradename SPARINE®), triflurpromazine (available under the tradename VESPRIN®), chlorprothixene (available under the tradename TARACTAN®), droperidol (available under the tradename INAPSINE®), acetophenazine (available under the tradename TINDAL®;), prochlorperazine (available under the tradename COMPAZINE®), methotrimeprazine (available under the tradename NOZINAN®), pipotiazine (available under the tradename PIPOTRIL®), ziprasidone, and hoperidone.

It will be appreciated by those skilled in the art that the compounds according to the invention may advantageously be used in conjunction with one or more other therapeutic agents, for instance, different antidepressant agents such as 5HT3 antagonists, serotonin agonists, NK-1 antagonists, selective serotonin reuptake inhibitors (SSRI), noradrenaline re-uptake inhibitors (SNRI), tricyclic antidepressants, dopaminergic antidepressants, H3 antagonists, 5HT1A antagonists, 5HT1B antagonists, 5HT1D antagonists, D1 agonists, M1 agonists and/or anticonvulsant agents, as well as atypical antipsychotic drugs and cognitive enhancers.

Suitable 5HT3 antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

Suitable anticonvulsant agents which may be used in combination of the compounds of the invention include for example divalproex, carbamazepine and diazepam.

Suitable atypical antipsychotic drugs which may be used in combination of the compounds of the invention include for example risperidone, olanzapine, ziprasidone, aripiprazole and clozapine.

It will be appreciated that the compounds of the combination or composition may be administered simultaneously (either in the same or different pharmaceutical formulations), separately or sequentially.

Possible formulations include those suitable for oral, sub-lingual, buccal, parenteral (for example, subcutaneous, intramuscular, or intravenous), rectal, topical and intranasal administration and in forms suitable for administration by inhalation or insufflation (either through the mouth or nose). The most suitable means of administration for a particular patient will depend on the nature and severity of the conditions being treated and on the nature of the active compound, but, where possible, oral administration is preferred.

Formulations suitable for oral administration may be provided as discrete units, such as tablets, capsules, cachets, or lozenges, each containing a predetermined amount of the active compound; as powders or granules; as solutions or suspensions in aqueous or non-aqueous liquids; or as oil-in-water or water-in-oil emulsions.

Formulations suitable for sublingual or buccal administration include lozenges comprising the active compound and, typically, a flavoured base, such as sugar and acacia or tragacanth and pastilles comprising the active compound in an inert base, such as gelatin and glycerin or sucrose and acacia.

Formulations suitable for parenteral administration typically comprise sterile aqueous solutions containing a predetermined concentration of the active compound; the solution is preferably isotonic with the blood of the intended recipient. Although such solutions are preferably administered intravenously, they may also be administered by subcutaneous or intramuscular injection.

Formulations suitable for rectal administration are preferably provided as unit-dose suppositories comprising the active ingredient and one or more solid carriers forming the suppository base, for example, cocoa butter.

Formulations suitable for topical or intranasal application include ointments, creams, lotions, pastes, gels, sprays, aerosols and oils. Suitable carriers for such formulations include petroleum jelly, lanolin, polyethylene glycols, alcohols, and combinations thereof.

The formulations of the invention may be prepared by any suitable method, typically by uniformly and intimately admixing the active compound(s) with liquids or finely divided solid carriers, or both, in the required proportions and then, if necessary, shaping the resulting mixture into the desired shape.

For example, a tablet may be prepared by compressing an intimate mixture comprising a powder or granules of the active ingredient and one or more optional ingredients, such as a binder, lubricant, inert diluent, or surface active dispersing agent, or by moulding an intimate mixture of powdered active ingredient and inert liquid diluent.

Aqueous solutions for parenteral administration are typically prepared by dissolving the active compound in sufficient water to give the desired concentration and then rendering the resulting solution sterile and isotonic.

It will be appreciated that the precise dose administered will depend on the age and condition of the patient and the frequency and route of administration and will be at the ultimate discretion of the attendant physician. The compound may be administered in single or divided doses and may be administered one or more times, for example 1 to 4 times per day.

A proposed dose of the active ingredient for use according to the invention for oral, sub-lingual, parenteral, buccal, rectal, intranasal or topical administration to a human (of approximately 70 kg bodyweight) for the treatment of neurological and neuropsychiatric disorders mediated by a GlyT1 inhibitor, including schizophrenia, may be about 1 to about 1000 mg, preferably about 5 to about 500 mg, more preferably about 10 to about 100 mg of the active ingredient per unit dose which could be administered, for example, 1 to 4 times per day.

Certain compounds which fall within the scope of formula (I) have not previously been described and are thus novel compounds.

Thus in a further aspect, the present invention provides
N-(4-fluoro-3-methylphenyl)-2-{3-[4-(methyloxy)phenyl]-2-oxo-1,4-diazaspiro[4.5]dec-3-en-1-yl}acetamide
N-[2-(methyloxy)phenyl]-2-[3-(4-methylphenyl)-2-oxo-1,4-diazaspiro[4.5]dec-3-en-1-yl]acetamide
2-[3-(4-chlorophenyl)-2-oxo-1,4-diazaspiro[4.5]dec-3-en-1-yl]-N-(2,3-difluorophenyl)acetamide
2-[3-(4-chlorophenyl)-2-oxo-1,4-diazaspiro[4.5]dec-3-en-1-yl]-N-(2-methylphenyl)acetamide
N-(2,4-dimethylphenyl)-2-[3-(4-methylphenyl)-2-oxo-1,4-diazaspiro[4.5]dec-3-en-1-yl]acetamide
N-(3,4-difluorophenyl)-2-[3-(4-methylphenyl)-2-oxo-1,4-diazaspiro[4.5]dec-3-en-1-yl]acetamide
N-(3-fluorophenyl)-2-[3-(4-methylphenyl)-2-oxo-1,4-diazaspiro[4.5]dec-3-en-1-yl]acetamide
N-(2-methylphenyl)-2-[3-(4-methylphenyl)-2-oxo-1,4-diazaspiro[4.5]dec-3-en-1-yl]acetamide
2-[3-(4-chlorophenyl)-2-oxo-1,4-diazaspiro[4.5]dec-3-en-1-yl]-N-(2,3-dimethylphenyl)acetamide
2-[3-(4-chlorophenyl)-2-oxo-1,4-diazaspiro[4.5]dec-3-en-1-yl]-N-phenylacetamide
N-(3-fluorophenyl)-2-{3-[4-(methyloxy)phenyl]-2-oxo-1,4-diazaspiro[4.5]dec-3-en-1-yl}acetamide
N-(2,4-dimethylphenyl)-2-{3-[4-(methyloxy)phenyl]-2-oxo-1,4-diazaspiro[4.5]dec-3-en-1-yl}acetamide
N-(3-chlorophenyl)-2-[3-(4-chlorophenyl)-2-oxo-1,4-diazaspiro[4.5]dec-3-en-1-yl]acetamide
N-(4-chloro-3-methylphenyl)-2-{3-[4-(methyloxy)phenyl]-2-oxo-1,4-diazaspiro[4.5]dec-3-en-1-yl}acetamide
2-{3-[4-(methyloxy)phenyl]-2-oxo-1,4-diazaspiro[4.5]dec-3-en-1-yl}-N-(2,3,4-trifluorophenyl)acetamide
or a solvate thereof.

The invention is further illustrated by the following non-limiting examples.

Abbreviations:

| | |
|---|---|
| THF | tetrahydrofuran |
| DCM | dichloromethane |
| DMF | dimethylformamide |
| HATU | O-(7-azabenzotriazol-1-yl)-N,N,N′,N′-tetramethyluroniumhexa |
| | fluorophosphate |
| NMP | N-methylpyrrolidinone |
| DIPEA | N,N-diisopropylethylamine |
| HOBt | 1-hydroxybenzotriazole hydrate |
| iPrOH | Isopropyl alcohol |

Analytical LC/MS chromatography conditions:

| | |
|---|---|
| Column: | Waters Atlantis 50 mm × 4.6 mm, 3 um particle |
| | size |
| Mobile phase: | A: 0.05% Formic acid + Water |
| | B: Acetonitrile + 0.05% Formic acid |
| Gradient: | 5-min runtime: 3% B to 97% B over 4 min |
| Flow rate: | 3 ml/min |
| UV wavelength range: | 220-330 nm |
| Temperature: | 30° C. |

Mass Directed Auto-Purification System chromatography conditions:

| | |
|---|---|
| Column: | Waters Atlantis 19 mm × 100 mm or 30 mm × 100 mm, |
| | 5 um particle size |
| Mobile phase: | A: 0.1% Formic acid + Water |
| | B: Acetonitrile + 0.1% Formic acid |
| Gradient: | 13.5 min runtime with 10 min gradient dependant on |
| | analytical retention time |
| Flow rate: | 20 or 40 ml/min |

General:

Where reactions are described as having been carried out in a similar manner to earlier, more completely described reactions, the general reaction conditions used were essentially the same. Work up conditions used were of the types standard in the art, but may have been adapted from one reaction to another.

### DESCRIPTIONS AND EXAMPLES

### Description 1

### 2-Chloro-N-(2-methoxyphenyl)acetamide

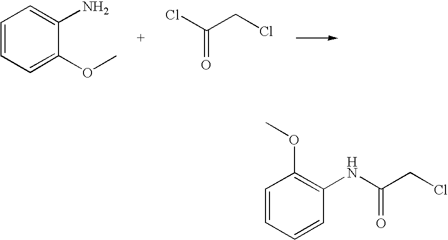

Chloroacetyl chloride (11.3 g, 100 mmol) was slowly added to 2-methoxyaniline (12.3 g, 100 mmol) in dioxan (100 mL), then the mixture was heated under reflux for 1 h. The solution was concentrated to 50 mL, cooled to room temperature, water was added; the solid was filtered off and crystallized from iPrOH+H₂O (1:1). The yield was 80-90%.

### Description 2

### 2-(4-Chlorophenyl)-2-oxoacetonitrile

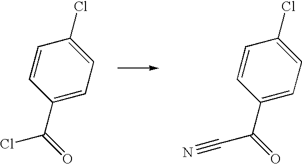

Mixture of 4-chlorobenzoyl chloride (87.5 g, 0.5 mol) and CuCN (53.7 g, 0.6 mol) was stirred for 2 h at 190° C. Then the product was distillate under vacuum (20 mm, 140-160° C.) to give a colourless fusible solid (60-70%).

### Description 3

### 2-Oxo-2-(4-chlorophenyl)thioacetamide

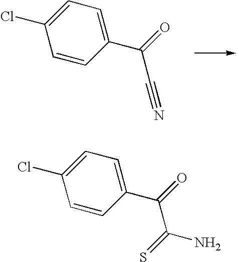

Hydrogen sulfide was bubbled through a solution of 2-(4-chlorophenyl)-2-oxoacetonitrile D2 (0.2 mol) and triethylamine (10 mL) in dry diethyl ether (400 mL) at r.t. for 2 h. The solvent was evaporated to give an orange solid (95%).

### Description 4

### 3-(4-Chlorophenyl)-1,4-diazaspiro[4.5]dec-3-ene-2-thione

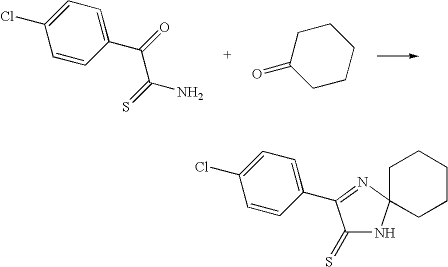

2-Oxo-2-(4-chlorophenyl)thioacetamide D3 (10 mmol), ammonium acetate (10 mmol), and cyclohexanone (10 mmol) in i-PrOH (50 mL) were heated under reflux for 1 h. The solution was cooled to room temperature. The yellow precipitate was collected by filtration, washed by i-PrOH. The yield was 70-75%.

### Description 5

### 3-(4-Chlorophenyl)-1,4-diazaspiro[4.5]dec-3-en-2-one

Method 1

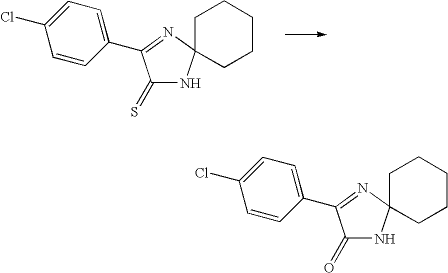

To solution of 20 mmol of NaOH, 10 mmol 3-(4-chlorophenyl)-1,4-diazaspiro[4.5]dec-3-ene-2-thione D4 in 150 mL of MeOH/H₂O (1:1) a solution 40 mmol of H₂O₂ (50% in wate slowly and accurately was added, then were stirred 1 h at r.t. White solid was filtered and crystallized from iPrOH+DMF. The yield was 60-65%.

Method 2

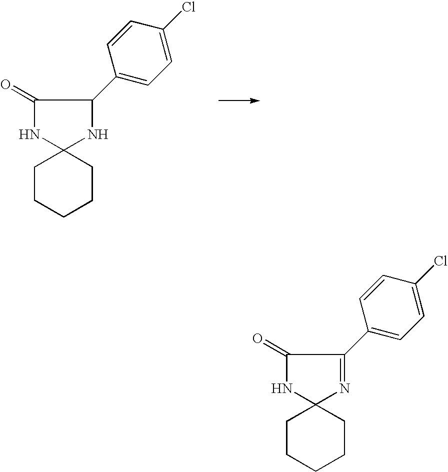

N-Bromosuccinimide (8.69 g; 48.81 mmol) was added in one portion to a stirred solution of 3-(4-chlorophenyl)-1,4-diazaspiro[4.5]decan-2-one D8 (12.91 g; 48.81 mmol) in DCM (400 ml) and the mixture stirred overnight at room temperature. Saturated aqueous sodium bicarbonate (500 ml) was added and the mixture stirred for 0.5 h. The layers were separated and the aqueous extracted with DCM (300 ml). The combined organics were dried (Na₂SO₄) and the solvent removed under reduced pressure at 45° C. The residual solid was partitioned between DCM (500 ml) and saturated aqueous sodium bicarbonate (500 ml) and stirred overnight at room temperature. The aqueous layer was extracted with DCM (300 ml) and the organic layers combined, dried (Na₂SO₄) and the solvent removed under reduced pressure to afford the title product (10.25 g; 80%) as a pale yellow solid. ¹H NMR (CDCl₃) δ: 1.51-1.70 (6H, m), 1.91-1.99 (4H, m), 7.42-7.49 (2H, m), 8.36-8.39 (2H, m), 8.88 (1H, s).

### Description 6

### Methyl amino(4-chlorophenyl)acetate

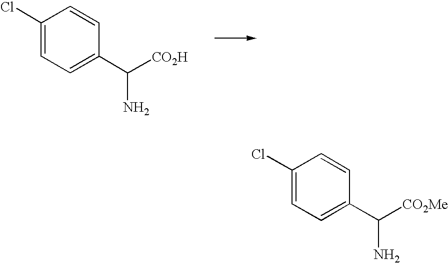

To ice-chilled methanol (300 ml) under argon was carefully added dropwise thionyl chloride (15.44 ml; 0.217 mol) over 45 min. 4-Chlorophenylglycine (26.26 g; 0.141 mol) was added, ice cooling removed and the reaction mixture warmed to 40° C.; the reaction was stirred at 40° C. for 48 h. After cooling to room temperature, the reaction was evaporated under reduced pressure. Re-evaporation from methanol afforded a white solid which was triturated with diethyl ether (ca. 700 ml) and then stored at ca. 4° C. for 64 h, filtered, washed with diethyl ether and dried in vacuo to afford the title product as the hydrochloride salt (33.40 g; 100%). ¹H NMR (d₆-DMSO) δ: 3.72 (3H, s), 5.36 (1H, s), 7.53-7.58 (4H, m), 9.07 (3H, s). Mass Spectrum (Electrospray LC/MS): Found 200 (MH⁺). C₉H₁₀³⁵ClNO₂ requires 199. Ret. time 1.32 min.

### Description 7

### 2-Amino-2-(4-chlorophenyl)acetamide

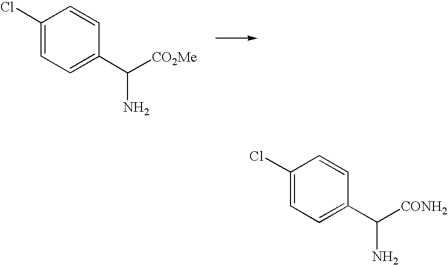

Methyl amino(4-chlorophenyl)acetate D6 as the hydrochloride salt (33.40 g; 0.14 mol) was dissolved in 0.88 ammonia (500 ml; ca. 7.4 mol) and stirred at room temperature for 64 h. The reaction mixture was extracted with DCM (300 ml×6), the extracts dried (Na₂SO₄) and evaporated under reduced pressure to a white solid, which was dried under reduced pressure to afford the title product (22.45 g; 86%). ¹H NMR (CDCl₃) δ: 1.82 (2H, br s), 4.53 (1H, s), 5.49 (1H, br s), 6.92 (1H, br s), 7.32-7.39 (4H, m).

### Description 8

### 3-(4-Chlorophenyl)-1,4-diazaspiro[4.5]decan-2-one

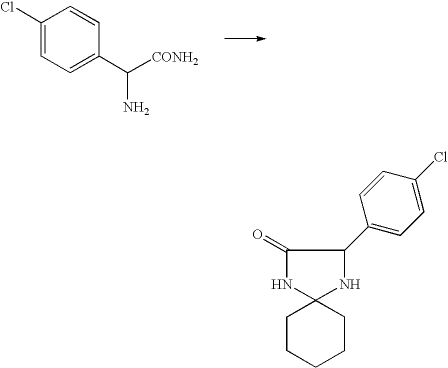

To 2-amino-2-(4-chlorophenyl)acetamide D7 (10.00 g; 54.3 mmol) in methanol (500 ml) was added cyclohexanone (5.62 ml; 54.3 mmol) and H—Y zeolites (10.00 g) and the mixture stirred under reflux for 24 h. The reaction was allowed to cool to room temperature and after 4 days was filtered and the solid washed well with methanol. The filtrate was evaporated to afford the title product (12.91 g; 90%) as a white solid. ¹H NMR (CDCl₃) δ: 1.44-1.57 (4H, m), 1.66-1.76 (6H, m), 2.21 (1H, s), 4.69 (1H, s), 6.80 (1H, s), 7.32-7.35 (2H, m), 7.45-7.49 (2H, m).

### Description 9

### Ethyl [3-(4-chlorophenyl)-2-oxo-1,4-diazaspiro[4.5]dec-3-en-1-yl]acetate

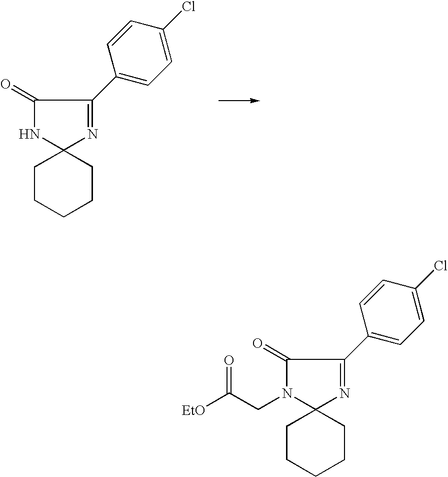

A mixture of 3-(4-chlorophenyl)-1,4-diazaspiro[4.5]dec-3-en-2-one D5 (4.06 g; 15.5 mmol), ethyl bromoacetate (8.55 ml; 77.3 mmol) and potassium carbonate (2.35 g; 17.0 mmol) in acetone (200 ml) was heated at reflux for ca.40 h, cooled and partitioned between water and DCM. The DCM layer was separated, evaporated and the residue chromatographed twice, eluting with ethyl acetate and ethyl acetate-pentane mixtures to afford the title product (3.2 g; 59%). ¹H NMR (CDCl₃) δ:1.24-1.34 (5H, m), 1.43-1.47 (2H, m), 1.74-1.82 (4H, m), 1.88-1.91 (1H, m), 1.97-2.07 (1H, m), 4.16 (2H, s), 4.20-4.27 (2H, m), 7.41-7.45 (2H, m), 8.43-8.46 (2H, m).

### Description 10

### [3-(4-Chlorophenyl)-2-oxo-1,4-diazaspiro[4.5]dec-3-en-1-yl]acetic acid

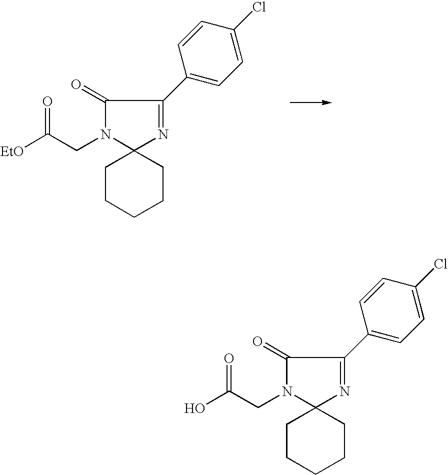

To a stirred mixture of ethyl [3-(4-chlorophenyl)-2-oxo-1,4-diazaspiro[4.5]dec-3-en-1-yl]acetate D9 (4.240 g; 12.17 mmol) in water (150 ml) and methanol (50 ml) was added sodium hydroxide (0.584 g; 14.6 mmol) and the reaction heated at 60° C. overnight, cooled and evaporated under reduced pressure. The residue was partitioned between water (400 ml) and ethyl acetate (200 ml). The aqueous layer was acidified with 5N HCl and extracted into DCM (150 ml×3). The combined DCM extracts were dried (Na₂SO₄) and evaporated under reduced pressure to afford the title acid (3.42 g; 88%) as a colourless solid. ¹H NMR (CDCl₃) δ: 1.25-1.35 (1H, m), 1.44-1.47 (2H, m), 1.76-2.07 (7H, m), 4.22 (2H, s), 7.10-7.70 (1H, br s), 7.41-7.44 (2H, m), 8.39-8.43 (2H, m).

### Description 11

### [3-(4-Chlorophenyl)-2-oxo-1,4-diazaspiro[4.5]dec-3-en-1-yl]acetyl chloride

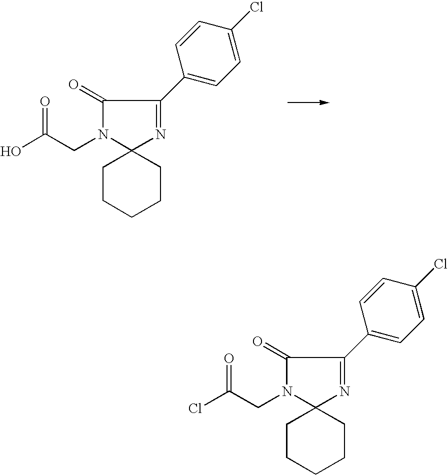

To a suspension of [3-(4-chlorophenyl)-2-oxo-1,4-diazaspiro[4.5]dec-3-en-1-yl]acetic acid D10 (300 mg; 0.936 mmol) in DCM (15 ml) was added oxalyl chloride (0.164 ml; 1.872 mmol) followed with stirring by DMF (1 drop). After stirring overnight the reaction was evaporated under reduced pressure to afford the title product (320 mg; 100%) as a pale yellow solid which was used without further purification. Mass Spectrum (Electrospray LC/MS; MeOH): Found 335 (MH⁺ for methyl ester). C₁₇H₁₉³⁵ClN₂O₃ requires 334. Ret. time 3.5 min.

### Description 12

### 2-Amino-2-[4-(methyloxy)phenyl]acetamide

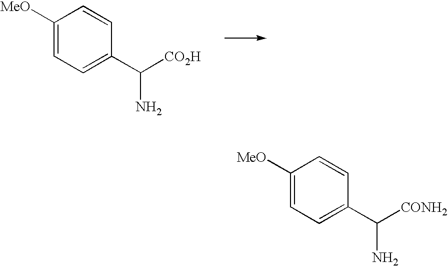

To an ice-cold suspension of 4-methoxyphenylglycine (3.77 g; 0.021 mol) in methanol was added thionyl chloride dropwise over 30 min. After complete addition, the reaction mixture was heated at reflux for 3 h, cooled and evaporated. The resulting solid was dissolved in 0.88 ammonia (100 ml) and stirred at room temperature overnight. The reaction was extracted twice with DCM and the organic phases separated with a Phase-Separation cartridge and evaporated under reduced pressure to afford the title product (0.45 g; 12%) as a white solid. ¹H NMR (CDCl₃) δ: 1.77 (2H, br s), 3.80 (3H, s), 4.50 (1H, s), 5.52 (1H, s), 6.83 (1H, s), 6.87-6.91 (2H, m), 7.33-7.36 (2H, m).

### Description 13

### 3-[4-(Methyloxy)phenyl]-1,4-diazaspiro[4.5]decan-2-one

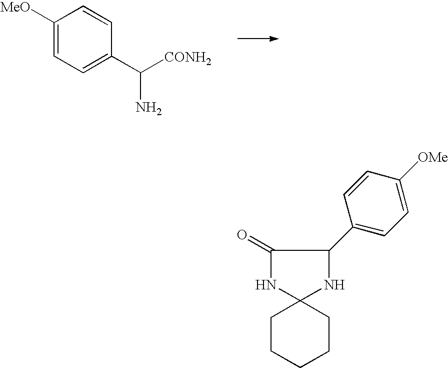

The title compound (0.420 g; 65%) was obtained from 2-amino-2-[4-(methyloxy)phenyl]acetamide D12 (0.450 g; 2.5 mmol), cyclohexanone (0.245 g; 2.5 mmol) and H—Y zeolites (1 g) in methanol (20 ml) in a similar manner to that described in D8. ¹H NMR (CDCl₃) δ: 1.44-1.57 (4H, m), 1.71-1.73 (6H, m), 2.11 (1H, br s), 3.80 (3H, s), 4.64 (1H, s), 6.55 (1H, br s), 6.89-6.92 (2H, m), 7.36-7.40 (2H, m).

### Description 14

### 3-[4-(Methyloxy)phenyl]-1,4-diazaspiro[4.5]dec-3-en-2-one

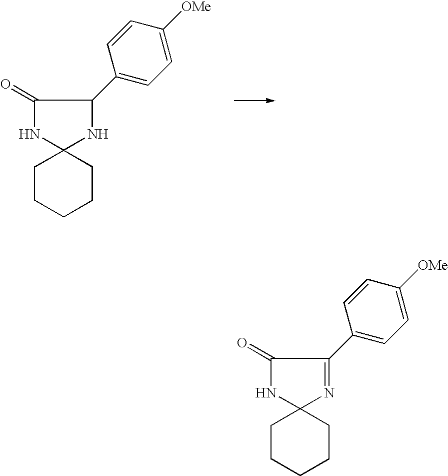

The title product (406 mg; 100%) was obtained from 3-[4-(methyloxy)phenyl]-1,4-diazaspiro[4.5]decan-2-one D13 (400 mg; 1.54 mmol) and N-bromosuccinimide (275 mg; 1.55 mmol) in DCM (20 ml) using a method similar to that described in D5 method 2. ¹H NMR (CDCl₃) δ: 1.40-1.75 (6H, m), 1.85-2.00 (4H, m), 3.87 (3H, s), 6.94-6.98 (2H, m), 8.18 (1H, br s), 8.37-8.40 (2H, m).

### Description 15

### Ethyl {3-[4-(methyloxy)phenyl]-2-oxo-1,4-diazaspiro[4.5]dec-3-en-1-yl}acetate

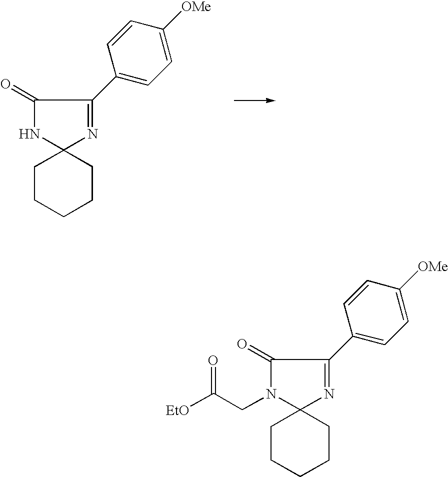

A mixture of 3-(4-methoxyphenyl)-1,4-diazaspiro[4.5]dec-3-en-2-one D14 (400 mg; 1.55 mmol), ethyl bromoacetate (0.7 ml; 6.30 mmol) and potassium carbonate (260 mg; 1.86 mmol) in acetone (200 ml) was heated at reflux for ca. 64 h; further ethyl bromoacetate (0.7 ml; 6.30 mmol) was added and heating continued for 48 h, after which further potassium carbonate (260 mg; 1.86 mmol) and ethyl bromoacetate (0.7 ml; 6.30 mmol) was added and heating continued for a further 48 h. Acetone was evaporated and the residue partitioned between DCM and water. The organic phase was separated and dried using a Phase-separation cartridge and evaporated, and the residue chromatographed eluting with ethyl acetate-pentane gradient to afford the title product (326 mg; 61%). ¹H NMR (CDCl₃) δ: 1.23-1.33 (4H, m), 1.42-1.45 (2H, m), 1.72-1.80 (4H, m), 1.87-1.90 (1H, s), 1.98-2.09 (2H, m), 3.87 (3H, s), 4.16 (2H, s), 4.19-4.25 (2H, s), 6.94-7.26 (2H, m), 8.45-8.48 (2H, m).

### Description 16

### {3-[4-(Methyloxy)phenyl]-2-oxo-1,4-diazaspiro[4.5]dec-3-en-1-yl}acetic acid

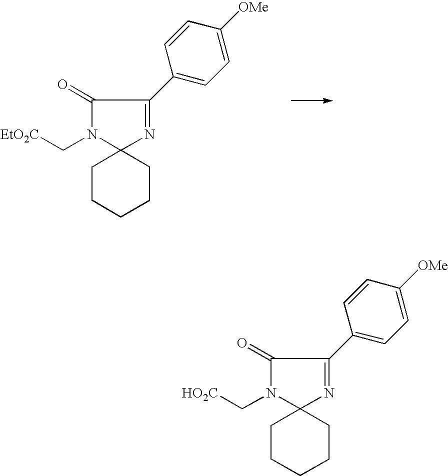

To ethyl {3-[4-(methyloxy)phenyl]-2-oxo-1,4-diazaspiro[4.5]dec-3-en-1-yl}acetate D15 (326 mg; 0.95 mmol) in ethanol (16 ml) and water (4 ml) was added sodium hydroxide (80 mg; 2.00 mmol) and the mixture heated at reflux for 16 h. The reaction was cooled and evaporated to give a slurry that was acidified with 2N HCl and extracted with ethyl acetate (×2). The organics were separated and dried with a little Na₂SO₄ and passed through a Phase-separation cartridge to afford the title product (290 mg; 97%). ¹H NMR (CDCl₃) δ: 1.20-1.33 (1H, m), 1.43-1.46 (2H, m), 1.74-1.80 (4H, m), 1.87-1.90 (1H, m), 1.98-2.18 (2H, m), 3.86 (3H, s), 4.22 (2H, s), 6.94-6.96 (2H, m), 8.42-8.44 (2H, m), 9.75 (1H, br s).

### Description 17

### {3-[4-(Methyloxy)phenyl]-2-oxo-1,4-diazaspiro[4.5]dec-3-en-1-yl}acetyl chloride

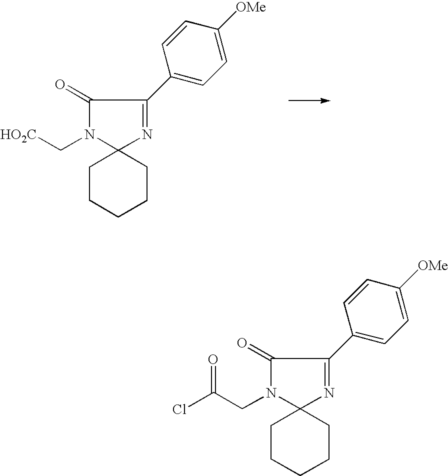

A mixture of {3-[4-(methyloxy)phenyl]-2-oxo-1,4-diazaspiro[4.5]dec-3-en-1-yl}acetic acid D16 (288 mg; 0.9 mmol), oxalyl chloride (0.18 ml; 2.0 mmol) and DMF (1 drop) in DCM (20 ml) was stirred at room temperature for 2 h. The reaction was evaporated and triturated with toluene (×2) to afford the title product as an orange solid which was used without further purification.

### Description 18

### 3-Phenyl-1,4-diazaspiro[4.5]decan-2-one

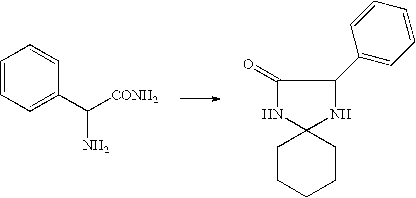

The title compound (1.24 g; 81%) was obtained from D-(−)-phenylglycinamide (1.00 g; 6.7 mmol), cyclohexanone (0.660 g; 6.7 mmol) and H—Y zeolites (2.00 g) in methanol (100 ml) in a similar manner to that described in D8. ¹H NMR (CDCl₃) δ: 1.44-1.57 (4H, m), 1.71-1.76 (6H, m), 2.17 (1H, br s), 4.70 (1H, s), 6.69 (1H, br s), 7.26-7.45 (3H, m), 7.49-7.50 (2H, m).

### Description 19

### 3-Phenyl-1,4-diazaspiro[4.5]dec-3-en-2-one

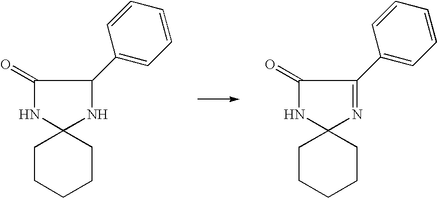

The title product (1.03 g; 100%) was obtained from 3-phenyl-1,4-diazaspiro[4.5]decan-2-one D18 (946 mg; 4.1 mmol) and N-bromosuccinimide (735 mg; 4.1 mmol) in DCM (20 ml) using a method similar to that described in D5 method 2. ¹H NMR (CDCl₃) δ: 1.52-1.70 (6H, m), 1.91-1.98 (4H, m), 7.44-7.53 (3H, m), 8.34 (1H, br s), 8.36-8.39 (2H, m).

### Description 20

### 2-Amino-2-(4-methylphenyl)acetamide

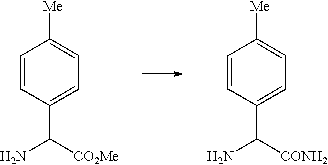

Methyl amino(4-methylphenyl)acetate hydrochloride (0.6 g, 2.54 mmol) was added portionwise over 10 minutes, with stirring to 0.880 ammonia (200 ml) and the solution stirred at room temperature overnight. The mixture was evaporated under reduced pressure, redissolved in 0.880 ammonia (20 ml) and extracted with DCM (4×15 ml). Combined organics were dried (Na₂SO₄) and evaporated under reduced pressure to afford a colourless solid (0.335 g, 73%). ¹H NMR (CDCl₃) δ: 1.80 (2H, br s), 2.34 (3H, s), 4.50 (1H, s), 5.67 (1H, br s), 6.83 (1H, br s), 7.15-7.32 (4H, m).

### Description 21

### 3-(4-Methylphenyl)-1,4-diazaspiro[4.5]decan-2-one

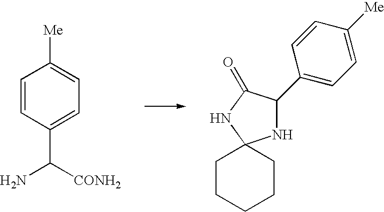

The title compound (0.404 g; 81%) was obtained from 2-amino-2-[4-(methyl)phenyl]acetamide D20 (0.335 g; 2.04 mmol), cyclohexanone (0.2 g; 2.04 mmol) and H—Y zeolites (0.09 g) in methanol (20 ml) in a similar manner to that described in D8. ¹H NMR (CDCl₃) δ: 1.35-1.60 (4H, m), 1.60-1.80 (6H, m), 2.10 (1H, br s), 2.34 (3H, s), 4.65 (1H, s), 7.16-7.20 (2H, m), 7.30-7.40 (3H, m).

### Description 22

### 3-(4-Methylphenyl)-1,4-diazaspiro[4.5]dec-3-en-2-one

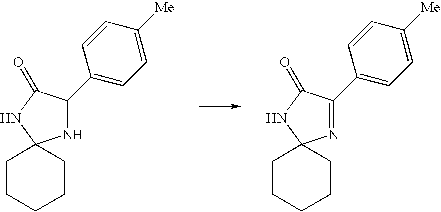

The title product (493 mg; >100%) was obtained from 3-[4-(methyl)phenyl]-1,4-diazaspiro[4.5]decan-2-one D21 (400 mg; 1.63 mmol) and N-bromosuccinimide (300 mg; 1.69 mmol) in DCM (20 ml) using a method similar to that described in D5 method 2. ¹H NMR (CDCl₃) δ: 1.40-1.75 (6H, m), 1.85-2.00 (4H, m), 2.41 (3H, s), 7.25-7.28 (2H, m), 8.27-8.30 (2H, m). 8.38 (1H, br s).

### Description 23

### Ethyl [3-(4-methylphenyl)-2-oxo-1,4-diazaspiro[4.5]dec-3-en-1-yl]acetate

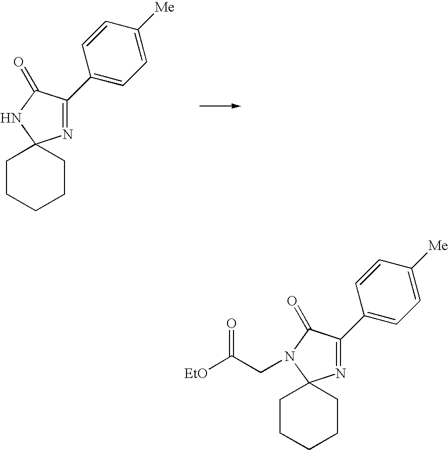

To a stirred solution of 3-(4-methylphenyl)-1,4-diazaspiro[4.5]dec-3-en-2-one D22 (0.493 g, 2.04 mmol), in anhydrous DMF (10 ml) at room temperature under argon was added 60% sodium hydride dispersed in oil (90 mg, 2.25 mmol), portion wise over 15 minutes. Stirring was continued for a further 15 minutes prior to the addition of ethylbromoacetate (0.374 g, 2.24 mmol) over 1 minute. The reaction mixture was stirred at room temperature for 2 hours, water added and the mixture extracted with diethyl ether. The organics were washed with water, brine, dried (Na₂SO₄) and evaporated under reduced pressure. The residue was purified by chromatography on silica gel eluting with 0-50% ethyl acetate in pentane gradient to afford the title compound as a colourless gum (470 mg, 70%). Mass Spectrum (Electrospray LC/MS; MeOH): Found 329 (MH⁺). C₁₉H₂₄N₂O₃ requires 328. Ret. time 3.57 min.

### Description 24

### [3-(4-Methylphenyl)-2-oxo-1,4-diazaspiro[4.5]dec-3-en-1-yl]acetic acid

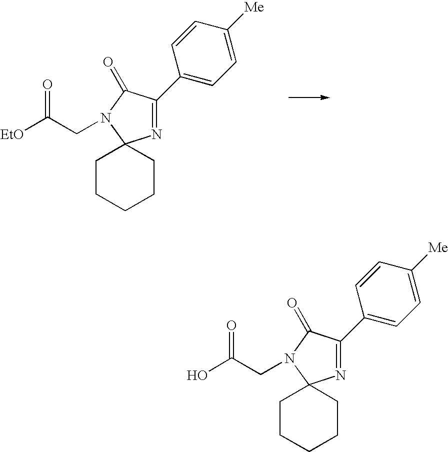

Sodium hydroxide (69 mg, 1.72 mmol) was added to ethyl [3-(4-methylphenyl)-2-oxo-1,4-diazaspiro[4.5]dec-3-en-1-yl]acetate D23 (470 mg, 1.43 mmol) in methanol (10 ml) and water (30 ml) and the mixture heated at 80° C. for 1.5 hours. On cooling the solution was evaporated under reduced pressure, water (50 ml) added, acidified with 5N hydrochloric acid and extracted with ethyl acetate (2×100 ml). Combined organic extracts were dried (Na₂SO₄) and evaporated under reduced pressure to give the title compound as a colourless solid (435 mg, 100%). Mass Spectrum (Electrospray LC/MS; MeOH): Found 301 (MH⁺). C₁₇H₂₀N₂O₃ requires 300. Ret. time 2.97 min.

### Description 25

### [3-(4-Methylphenyl)-2-oxo-1,4-diazaspiro[4.5]dec-3-en-1-yl]acetyl chloride

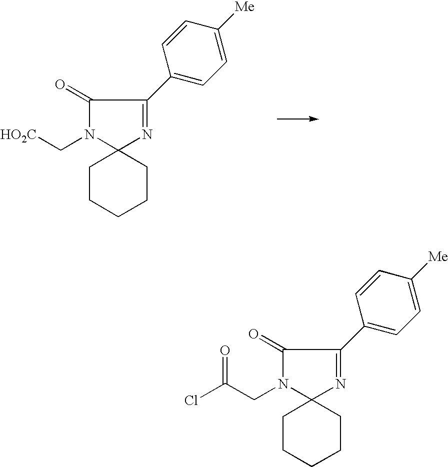

A mixture of [3-(4-methylphenyl)-2-oxo-1,4-diazaspiro[4.5]dec-3-en-1-yl]acetic acid D24 (426 mg; 1.42 mmol), oxalyl chloride (396 mg; 3.12 mmol) and DMF (1 drop) in DCM (20 ml) was stirred at room temperature for 18 h. The reaction was evaporated to afford the title product (424 mg; 94%) which was used without further purification.

### Example 1

### 2-[3-(4-Chlorophenyl)-2-oxo-1,4-diazaspiro[4.5]dec-3-en-1-y]-N-(2-methoxyphenyl)acetamide

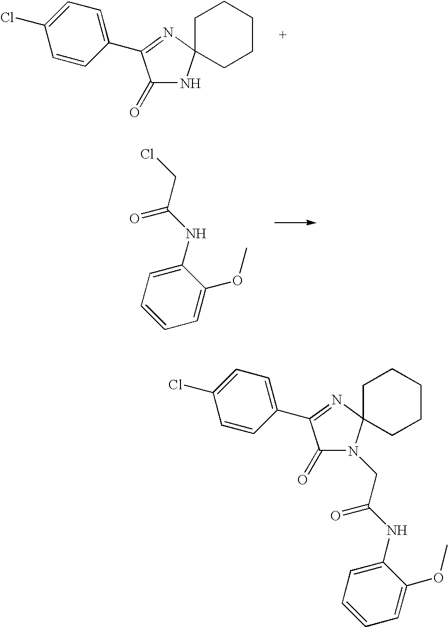

To solution of 10 mmol of 3-(4-chlorophenyl)-1,4-diazaspiro[4.5]dec-3-en-2-one D5, 10 mmol NaH in 30 mL of dry DMF a 10 mmol of 2-chloro-N-(2-methoxyphenyl)acetamide D1 was added, then mixture were stirred 1 h at 60° C. After cooling, water was added; the solid was filtered and crystallized from i-PrOH. The yield was 75-80%. MS m/z 426/428 MH⁺, C₂₃H24³⁵ClN₃O₃ requires 425, ¹H NMR (400 MHz, CDCl₃) δ ppm 1.25-1.45 (m, 3H), 1.75-1.95 (m, 3H), 2.05 (br m, 4H), 3.80 (s, 3H), 4.26 (s, 2H), 6.84 (dd, 1H), 6.92 (t, 1H), 7.04 (t, 1H), 7.45 (dd, 2H), 8.23 (dd, 1H), 7.45 (dd, 2H), 8.64 (br s, 1H).

The examples in Table 1 were prepared from the appropriate 3-(substitutedphenyl)-1,4-diazaspiro[4.5]dec-3-en-2-one (which was synthesized using methods analogous to those described in D1 to D4 and D5 method 1) using methods similar to Example 1.

TABLE 1

| Example | Strucure | Name |
|---|---|---|
| 2 | 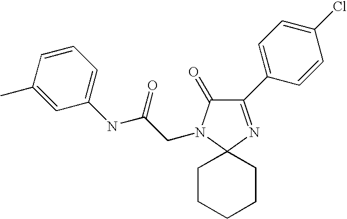 | 2-[3-(4-chlorophenyl)-2-oxo-1,4-diazaspiro[4.5]dec-3-en-1-yl]-N-(3-methylphenyl)acetamide |
| 3 | 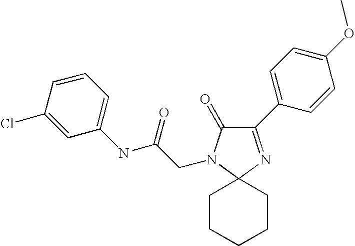 | N-(3-chlorophenyl)-2-{3-[4-(methyloxy)phenyl]-2-oxo-1,4-diazaspiro[4.5]dec-3-en-1-yl}acetamide |
| 4 | 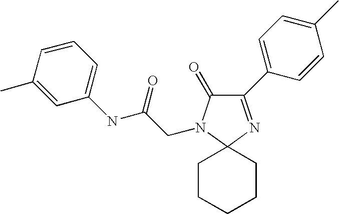 | N-(3-methylphenyl)-2-[3-(4-methylphenyl)-2-oxo-1,4-diazaspiro[4.5]dec-3-en-1-yl]acetamide |
| 5 | 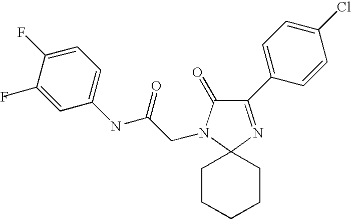 | 2-[3-(4-chlorophenyl)-2-oxo-1,4-diazaspiro[4.5]dec-3-en-1-yl]-N-(3,4-difluorophenyl)acetamide |
| 6 | 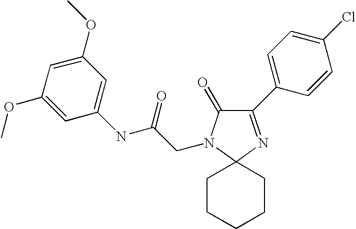 | N-[3,5-bis(methyloxy)phenyl]-2-[3-(4-chlorophenyl)-2-oxo-1,4-diazaspiro[4.5]dec-3-en-1-yl]acetamide |
| 7 | 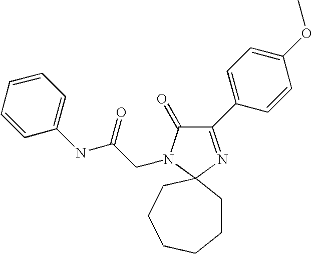 | 2-{3-[4-(methyloxy)phenyl]-2-oxo-1,4-diazaspiro[4.6]undec-3-en-1-yl}-N-phenylacetamide |
| 8 | 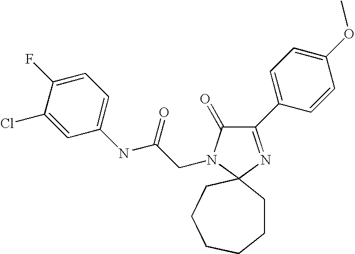 | N-(3-chloro-4-fluorophenyl)-2-{3-[4-(methyloxy)phenyl]-2-oxo-1,4-diazaspiro[4.6]undec-3-en-1-yl}acetamide |
| 9 | 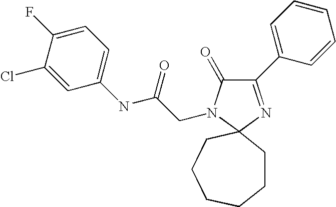 | N-(3-chloro-4-fluorophenyl)-2-(2-oxo-3-phenyl-1,4-diazaspiro[4.6]undec-3-en-1-yl)acetamide |
| 10 | 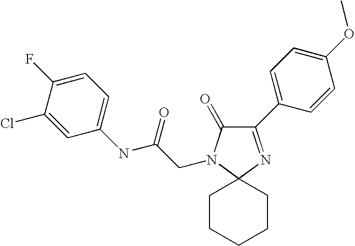 | N-(3-chloro-4-fluorophenyl)-2-{3-[4-(methyloxy)phenyl]-2-oxo-1,4-diazaspiro[4.5]dec-3-en-1-yl}acetamide |
| 11 | 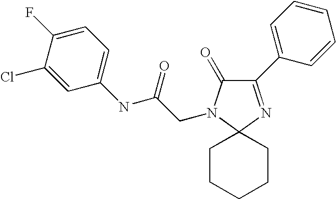 | N-(3-chloro-4-fluorophenyl)-2-(2-oxo-3-phenyl-1,4-diazaspiro[4.5]dec-3-en-1-yl)acetamide |
| 12 | 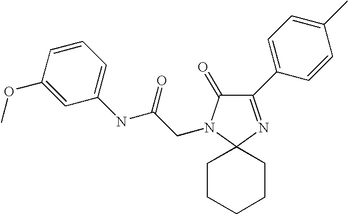 | N-[3-(methyloxy)phenyl]-2-[3-(4-methylphenyl)-2-oxo-1,4-diazaspiro[4.5]dec-3-en-1-yl]acetamide |
| 13 | 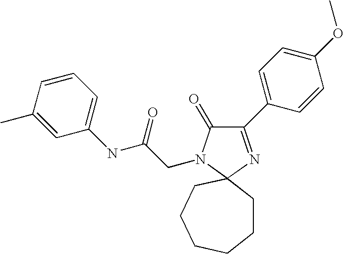 | 2-{3-[4-(methyloxy)phenyl]-2-oxo-1,4-diazaspiro[4.6]undec-3-en-1-yl}-N-(3-methylphenyl)acetamide |
| 14 | 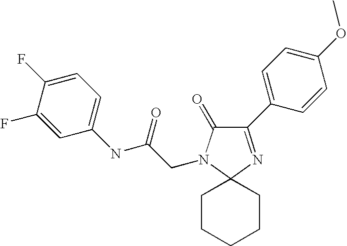 | N-(3,4-difluorophenyl)-2-{3-[4-(methyloxy)phenyl]-2-oxo-1,4-diazaspiro[4.5]dec-3-en-1-yl}acetamide |
| 15 | 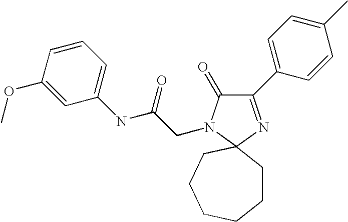 | N-[3-(methyloxy)phenyl]-2-[3-(4-methylphenyl)-2-oxo-1,4-diazaspiro[4.6]undec-3-en-1-yl]acetamide |
| 16 | 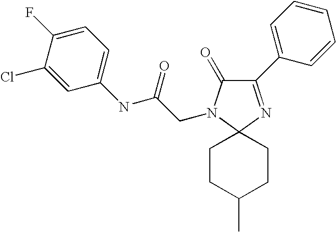 | N-(3-chloro-4-fluorophenyl)-2-(8-methyl-2-oxo-3-phenyl-1,4-diazaspiro[4.5]dec-3-en-1-yl)acetamide |
| 17 | 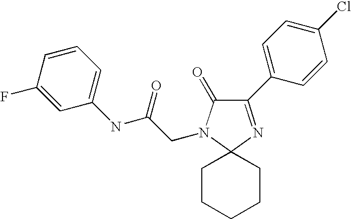 | 2-[3-(4-chlorophenyl)-2-oxo-1,4-diazaspiro[4.5]dec-3-en-1-yl]-N-(3-fluorophenyl)acetamide |
| 18 | 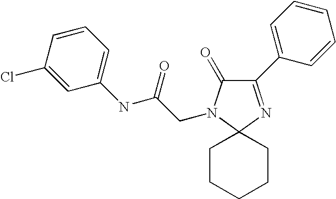 | N-(3-chlorophenyl)-2-(2-oxo-3-phenyl-1,4-diazaspiro[4.5]dec-3-en-1-yl)acetamide |
| 19 | 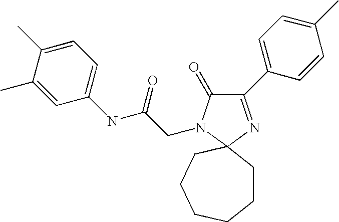 | N-(3,4-dimethylphenyl)-2-[3-(4-methylphenyl)-2-oxo-1,4-diazaspiro[4.6]undec-3-en-1-yl]acetamide |
| 20 | 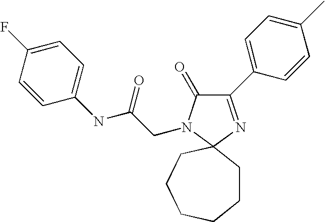 | N-(4-fluorophenyl)-2-[3-(4-methylphenyl)-2-oxo-1,4-diazaspiro[4.6]undec-3-en-1-yl]acetamide |
| 21 | 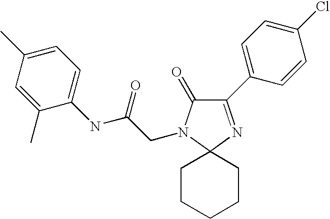 | 2-[3-(4-chlorophneyl)-2-oxo-1,4-diazaspiro[4.5]dec-3-en-1-yl]-N-(2,4-dimethylphenyl)acetamide |
| 22 | 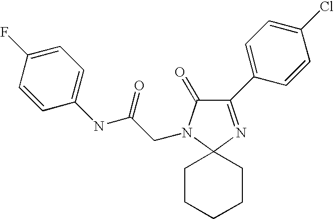 | 2-[3-(4-chlorophenyl)-2-oxo-1,4-diazaspiro[4.5]dec-3-en-1-yl]-N-(4-fluorophenyl)acetamide |
| 23 | 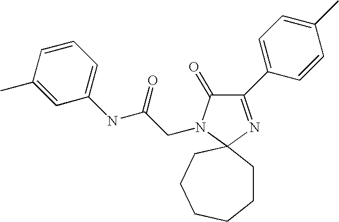 | N-(3-methylphenyl)-2-[3-(4-methylphenyl)-2-oxo-1,4-diazaspiro[4.6]undec-3-en-1-yl]acetamide |
| 24 | 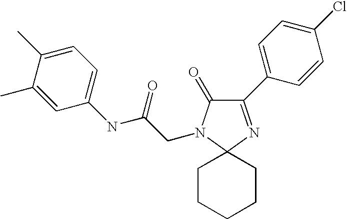 | 2-[3-(4-chlorophenyl)-2-oxo-1,4-diazaspiro[4,5]dec-3-en-1-yl]-N-(3,4-dimethylphenyl)acetamide |
| 25 | 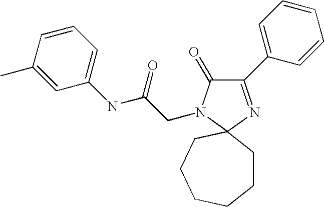 | N-(3-methylphenyl)-2-(2-oxo-3-phenyl-1,4-diazaspiro[4.6]undec-3-en-1-yl)acetamide |
| 26 | 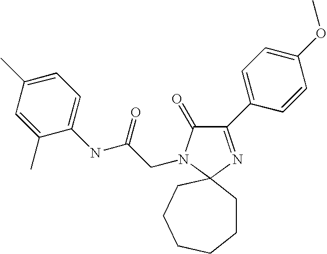 | N-(2,4-dimethylphenyl)-2-{3-[4-(methyloxy)phenyl]-2-oxo-1,4-diazaspiro[4.6]undec-3-en-1-yl}acetamide |
| 27 | 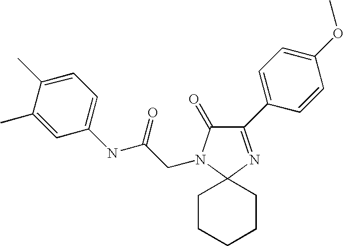 | N-(3,4-dimethylphenyl)-2-{3-[4-(methyloxy)phenyl]-2-oxo-1,4-diazaspiro[4.5]dec-3-en-1-yl}acetamide |
| 28 | 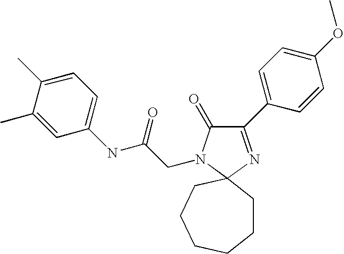 | N-(3,4-dimethylphenyl)-2-{3-[4-(methyloxy)phenyl]-2-oxo-1,4-diazaspiro[4.6]undec-3-en-1-yl}acetamide |
| 29 | 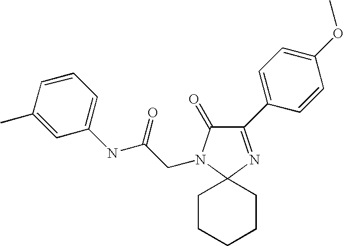 | 2-{3-[4-(methyloxy)phenyl]-2-oxo-1,4-diazaspiro[4.5]dec-3-en-1-yl}-N-(3-methylphenyl)acetamide |
| 30 | 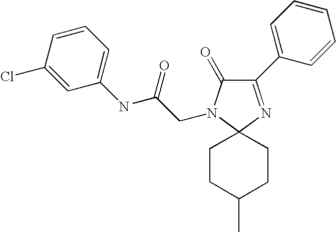 | N-(3-chlorophenyl)-2-(8-methyl-2-oxo-3-phenyl-1,4-diazaspiro[4.5]dec-3-en-1-yl)acetamide |
| 31 | 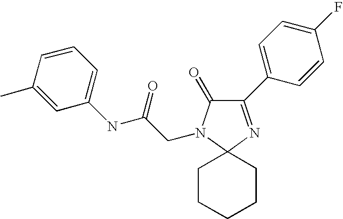 | 2-[3-(4-fluorophenyl)-2-oxo-1,4-diazaspiro[4.5]dec-3-en-1-yl]-N-(3-methylphenyl)acetamide |
| 32 | 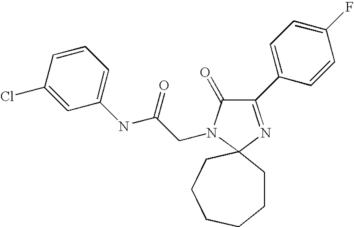 | N-(3-chlorophenyl)-2-[3-(4-fluorophenyl)-2-oxo-1,4-diazaspiro[4.6]undec-3-en-1-yl]acetamide |
| 33 | 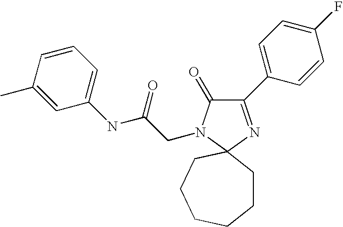 | 2-[3-(4-fluorophenyl)-2-oxo-1,4-diazaspiro[4.6]undec-3-en-1-yl]-N-(3-methylphenyl)acetamide |
| 34 | 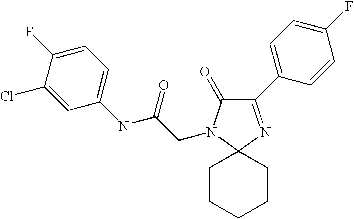 | N-(3-chloro-4-fluorophenyl)-2-[3-(4-fluorophenyl)-2-oxo-1,4-diazaspiro[4.5]dec-3-en-1-yl]acetamide |
| 35 | 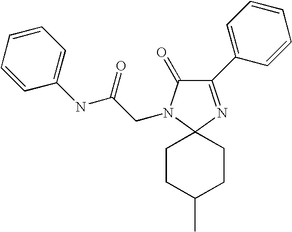 | 2-(8-methyl-2-oxo-3-phenyl-1,4-diazaspiro[4.5]dec-3-en-1-yl)-N-phenylacetamide |
| 36 | 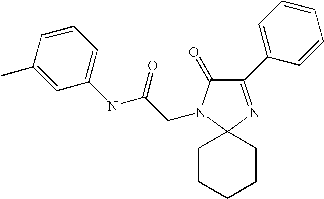 | N-(3-methylphenyl)-2-(2-oxo-3-phenyl-1,4-diazaspiro[4.5]dec-3-en-1-yl)acetamide |
| 37 | 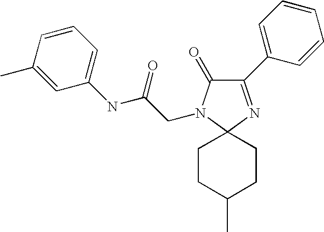 | 2-(8-methyl-2-oxo-3-phenyl-1,4-diazaspiro[4.5]dec-3-en-1-yl)-N-(3-methylphenyl)acetamide |
| 38 | 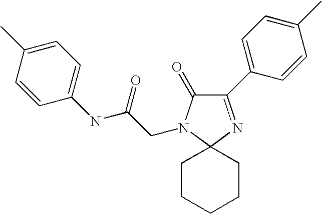 | N-(4-methylphenyl)-2-[3-(4-methylphenyl)-2-oxo-1,4-diazaspiro[4.5]dec-3-en-1-yl]acetamide |
| 39 | 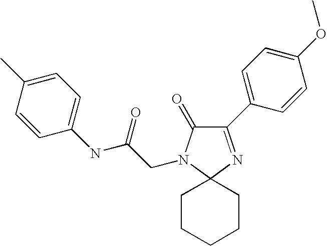 | 2-{3-[4-(methyloxy)phenyl]-2-oxo-1,4-diazaspiro[4.5]dec-3-en-1-yl}-N-(4-methylphenyl)acetamide |
| 40 | 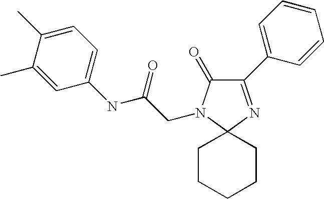 | N-(3,4-dimethylphenyl)-2-(2-oxo-3-phenyl-1,4-diazaspiro[4.5]dec-3-en-1-yl)acetamide |
| 41 | 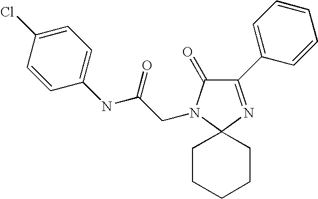 | N-(4-chlorophenyl)-2-(2-oxo-3-phenyl-1,4-diazaspiro[4.5]dec-3-en-1-yl)acetamide |
| 42 | 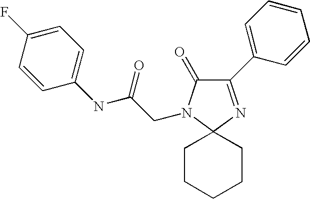 | N-(4-fluorophenyl)-2-(2-oxo-3-phenyl-1,4-diazaspiro[4.5]dec-3-en-1-yl)acetamide |
| 43 | 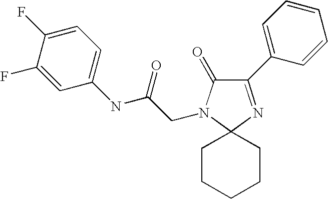 | N-(3,4-difluorophenyl)-2-(2-oxo-3-phenyl-1,4-diazaspiro[4.5]dec-3-en-1-yl)acetamide |
| 44 | 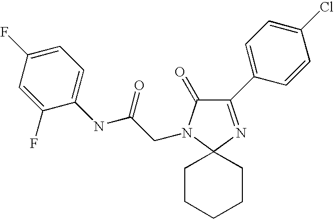 | 2-[3-(4-chlorophenyl)-2-oxo-1,4-diazaspiro[4.5]dec-3-en-1-yl]-N-(2,4-difluorophenyl)acetamide |
| 45 | 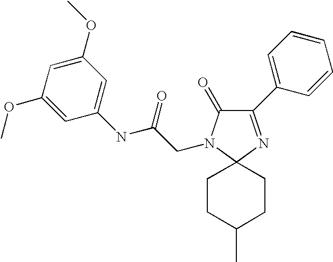 | N-[3,5-bis(methyloxy)phenyl]-2-(8-methyl-2-oxo-3-phenyl-1,4-diazaspiro[4.5]dec-3-en-1-yl)acetamide |
| 46 | 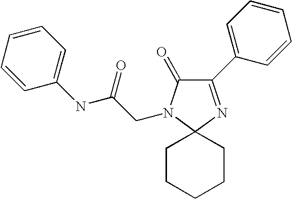 | 2-(2-oxo-3-phenyl-1,4-diazaspiro[4.5]dec-3-en-1-yl)-N-phenylacetamide |
| 47 | 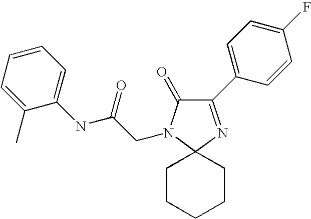 | 2-[3-(4-fluorophenyl)-2-oxo-1,4-diazaspiro[4.5]dec-3-en-1-yl]-N-(2-methylphenyl)acetamide |
| 48 | 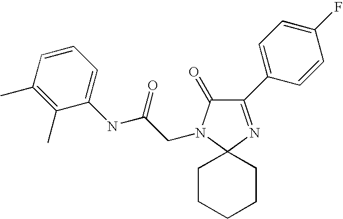 | N-(2,3-dimethylphenyl)-2-[3-(4-fluorophenyl)-2-oxo-1,4-diazaspiro[4.5]dec-3-en-1-yl]acetamide |
| 49 | 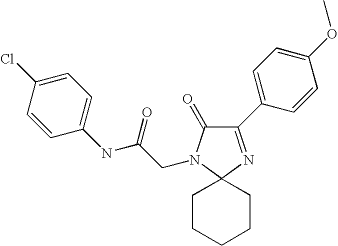 | N-(4-chlorophenyl)-2-{3-[4-(methyloxy)phenyl]-2-oxo-1,4-diazaspiro[4.5]dec-3-en-1-yl}acetamide |
| 50 | 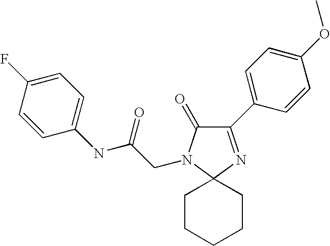 | N-(4-fluorophenyl)-2-{3-[4-(methyloxy)phenyl]-2-oxo-1,4-diazaspiro[4.5]dec-3-en-1-yl}acetamide |
| 51 | 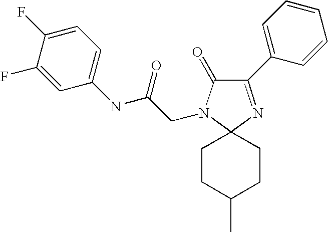 | N-(3,4-difluorophenyl)-2-(8-methyl-2-oxo-3-phenyl-1,4-diazaspiro[4.5]dec-3-en-1-yl)acetamide |
| 52 | 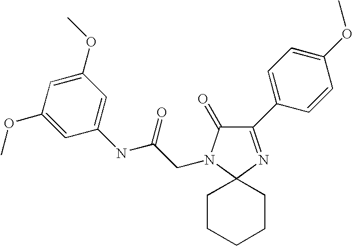 | N-[3,5-bis(methyloxy)phenyl]-2-{3-[4-(methyloxy)phenyl]-2-oxo-1,4-diazaspiro[4.5]dec-3-en-1-yl}acetamide |
| 53 | 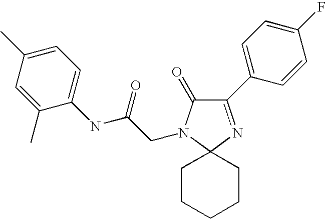 | N-(2,4-dimethylphenyl)-2-[3-(4-fluorophenyl)-2-oxo-1,4-diazaspiro[4.5]dec-3-en-1-yl]acetamide |
| 54 | 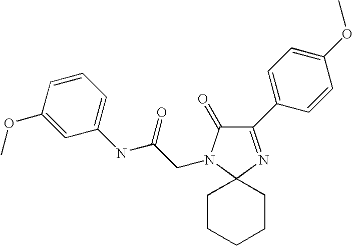 | N-[3-(methyloxy)phenyl]-2-{3-[4-(methyloxy)phenyl]-2-oxo-1,4-diazaspiro[4.5]dec-3-en-1-yl}acetamide |
| 55 | 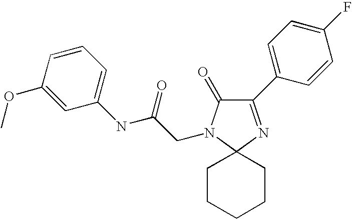 | 2-[3-(4-fluorophenyl)-2-oxo-1,4-diazaspiro[4.5]dec-3-en-1-yl]-N-[3-(methyloxy)phenyl]acetamide |
| 56 | 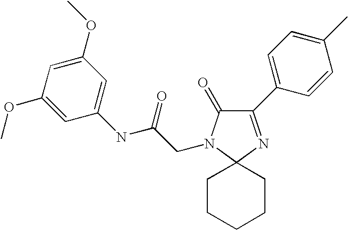 | N-[3,5-bis(methyloxy)phenyl]-2-[3-(4-methylphenyl)-2-oxo-1,4-diazaspiro[4.5]dec-3-en-1-yl]acetamide |
| 57 | 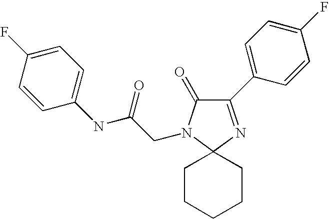 | N-(4-fluorophenyl)-2-[3-(4-fluorophenyl)-2-oxo-1,4-diazaspiro[4.5]dec-3-en-1-yl]acetamide |
| 58 | 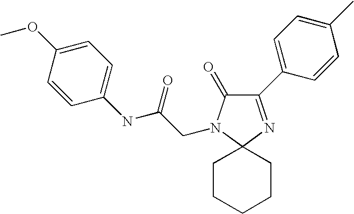 | N-[4-(methyloxy)phenyl]-2-[3-(4-methylphenyl)-2-oxo-1,4-diazaspiro[4.5]dec-3-en-1-yl]acetamide |
| 59 | 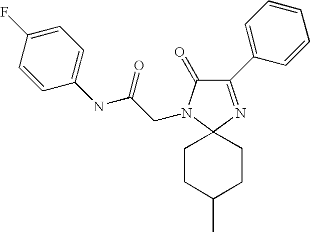 | N-(4-fluorophenyl)-2-(8-methyl-2-oxo-3-phenyl-1,4-diazaspiro[4.5]dec-3-en-1-yl)acetamide |
| 60 | 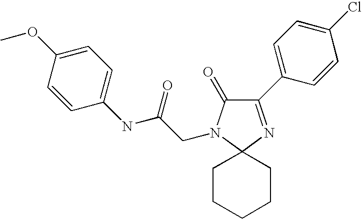 | 2-[3-(4-chlorophenyl)-2-oxo-1,4-diazaspiro[4.5]dec-3-en-1-yl]-N-[4-(methyloxy)phenyl]acetamide |
| 61 | 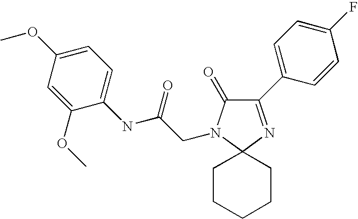 | N-[2,4-bis(methyloxy)phenyl]-2-[3-(4-fluorophenyl)-2-oxo-1,4-diazaspiro[4.5]dec-3-en-1-yl]acetamide |
| 62 | 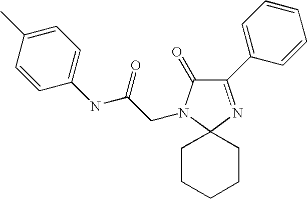 | N-(4-methylphenyl)-2-(2-oxo-3-phenyl-1,4-diazaspiro[4.5]dec-3-en-1-yl)acetamide |
| 63 | 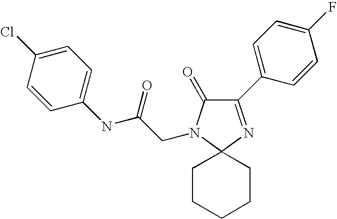 | N-(4-chlorophenyl)-2-[3-(4-fluorophenyl)-2-oxo-1,4-diazaspiro[4.5]dec-3-en-1-yl]acetamide |
| 64 | 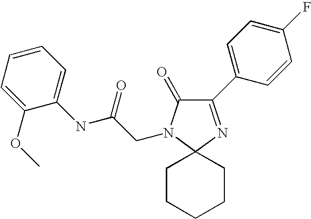 | 2-[3-(4-fluorophenyl)-2-oxo-1,4-diazaspiro[4.5]dec-3-en-1-yl]-N-[2-(methyloxy)phenyl]acetamide |
| 65 | 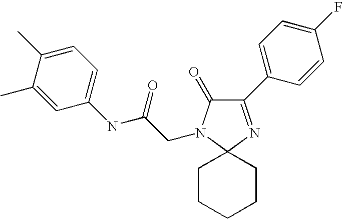 | N-(3,4-dimethylphenyl)-2-[3-(4-fluorophenyl)-2-oxo-1,4-diazaspiro[4.5]dec-3-en-1-yl]acetamide |
| 66 | 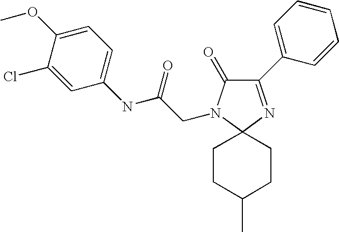 | N-[3-chloro-4-(methyloxy)phenyl]-2-(8-methyl-2-oxo-3-phenyl-1,4-diazaspiro[4.5]dec-3-en-1-yl)acetamide |
| 67 | 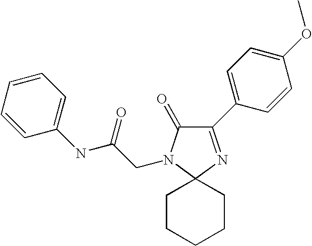 | 2-{3-[4-(methyloxy)phenyl]-2-oxo-1,4-diazaspiro[4.5]dec-3-en-1-yl}-N-phenylacetamide |
| 68 | 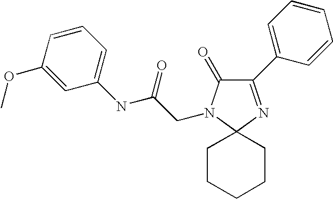 | N-[3-(methyloxy)phenyl]-2-(2-oxo-3-phenyl-1,4-diazaspiro[4.5]dec-3-en-1-yl)acetamide |
| 69 | 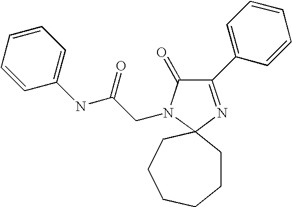 | 2-(2-oxo-3-phenyl-1,4-diazaspiro[4.6]undec-3-en-1-yl)-N-phenylacetamide |
| 70 | 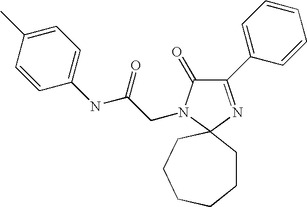 | N-(4-methylphenyl)-2-(2-oxo-3-phenyl-1,4-diazaspiro[4.6]undec-3-en-1-yl)acetamide |
| 71 | 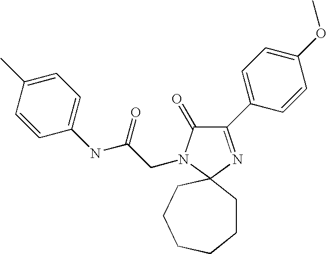 | 2-{3-[4-(methyloxy)phenyl]-2-oxo-1,4-diazaspiro[4.6]undec-3-en-1-yl}-N-(4-methylphenyl)acetamide |
| 72 | 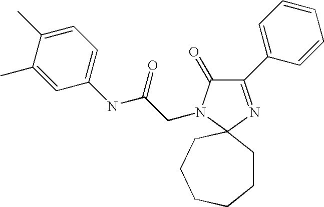 | N-(3,4-dimethylphenyl)-2-(2-oxo-3-phenyl-1,4-diazaspiro[4.6]undec-3-en-1-yl)acetamide |
| 73 | 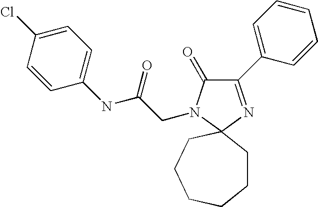 | N-(4-chlorophenyl)-2-(2-oxo-3-phenyl-1,4-diazaspiro[4.6]undec-3-en-1-yl)acetamide |
| 74 | 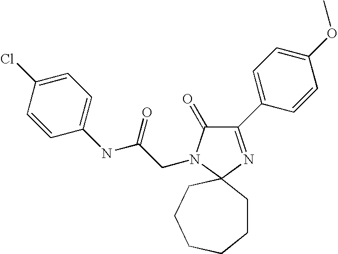 | N-(4-chlorophenyl)-2-{3-[4-(methyloxy)phenyl]-2-oxo-1,4-diazaspiro[4.6]undec-3-en-1-yl}acetamide |
| 75 | 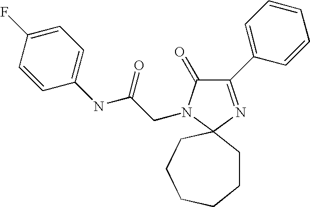 | N-(4-fluorophenyl)-2-(2-oxo-3-phenyl-1,4-diazaspiro[4.6]undec-3-en-1-yl)acetamide |
| 76 | 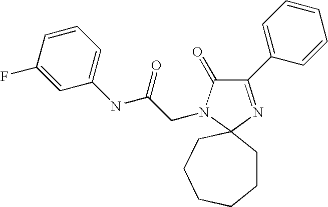 | N-(3-fluorophenyl)-2-(2-oxo-3-phenyl-1,4-diazaspiro[4.6]undec-3-en-1-yl)acetamide |
| 77 | 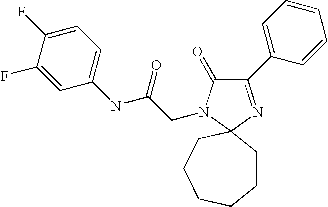 | N-(3,4-difluorophenyl)-2-(2-oxo-3-phenyl-1,4-diazaspiro[4.6]undec-3-en-1-yl)acetamide |
| 78 | 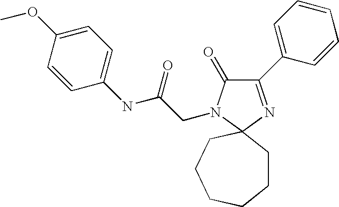 | N-[4-(methyloxy)phenyl]-2-(2-oxo-3-phenyl-1,4-diazaspiro[4.6]undec-3-en-1-yl)acetamide |
| 79 | 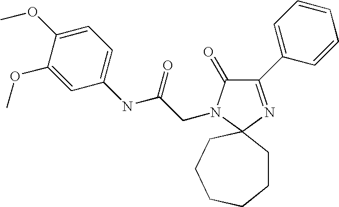 | N-[3,4-bis(methyloxy)phenyl]-2-(2-oxo-3-phenyl-1,4-diazaspiro[4.6]undec-3-en-1-yl)acetamide |
| 80 | 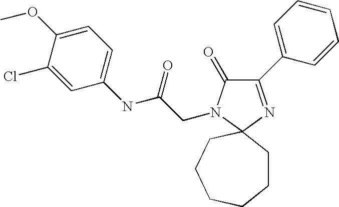 | N-[3-chloro-4-(methyloxy)phenyl]-2-(2-oxo-3-phenyl-1,4-diazaspiro[4.6]undec-3-en-1-yl)acetamide |
| 81 | 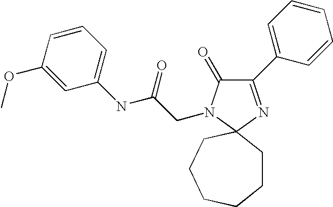 | N-[3-(methyloxy)phenyl]-2-(2-oxo-3-phenyl-1,4-diazaspiro[4.6]undec-3-en-1-yl)acetamide |
| 82 | 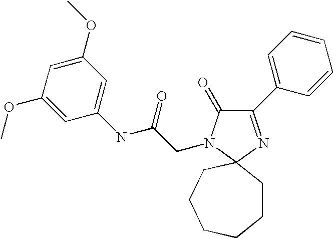 | N-[3,5-bis(methyloxy)phenyl]-2-(2-oxo-3-phenyl-1,4-diazaspiro[4.6]undec-3-en-1-yl)acetamide |
| 83 | 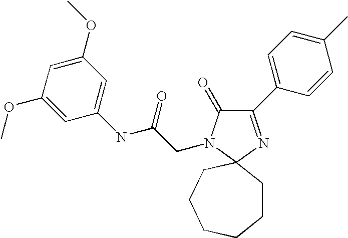 | N-[3,5-bis(methyloxy)phenyl]-2-[3-(4-methylphenyl)-2-oxo-1,4-diazaspiro[4.6]undec-3-en-1-yl]acetamide |
| 84 | 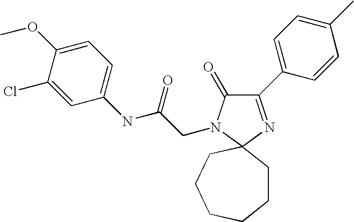 | N-[3-chloro-4-(methyloxy)phenyl]-2-[3-(4-methylphenyl)-2-oxo-1,4-diazaspiro[4.6]undec-3-en-1-yl]acetamide |
| 85 | 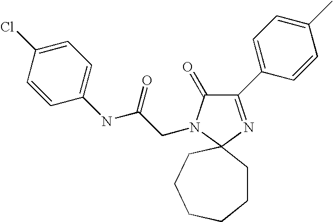 | N-(4-chlorophenyl)-2-[3-(4-methylphenyl)-2-oxo-1,4-diazaspiro[4.6]undec-3-en-1-yl]acetamide |
| 86 | 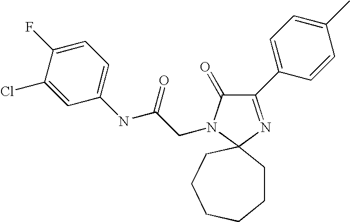 | N-(3-chloro-4-fluorophenyl)-2-[3-(4-methylphenyl)-2-oxo-1,4-diazaspiro[4.6]undec-3-en-1-yl]acetamide |
| 87 | 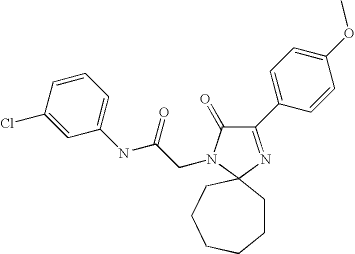 | N-(3-chlorophenyl)-2-{3-[4-(methyloxy)phenyl]-2-oxo-1,4-diazaspiro[4.6]undec-3-en-1-yl}acetamide |
| 88 | 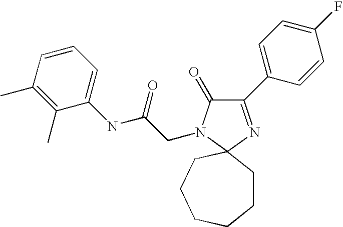 | N-(2,3-dimethylphenyl)-2-[3-(4-fluorophenyl)-2-oxo-1,4-diazaspiro[4.6]undec-3-en-1-yl]acetamide |
| 89 | 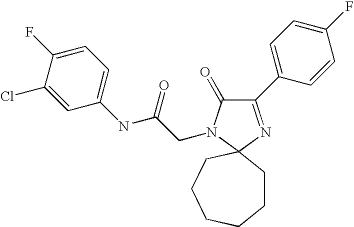 | N-(3-chloro-4-fluorophenyl)-2-[3-(4-fluorophenyl)-2-oxo-1,4-diazaspiro[4.6]undec-3-en-1-yl]acetamide |
| 90 | 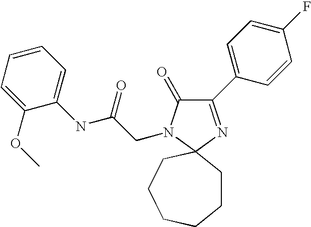 | 2-[3-(4-fluorophenyl)-2-oxo-1,4-diazaspiro[4.6]undec-3-en-1-yl]-N-[2-(methyloxy)phenyl]acetamide |
| 91 | 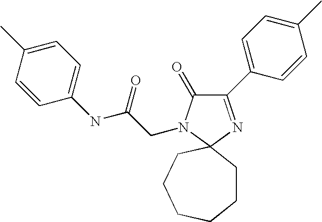 | N-(4-methylphenyl)-2-[3-(4-methylphenyl)-2-oxo-1,4-diazaspiro[4.6]undec-3-en-1-yl]acetamide |
| 92 | 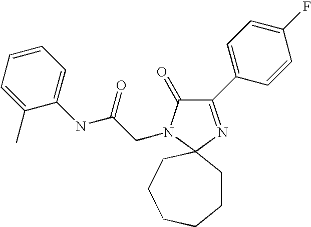 | 2-[3-(4-fluorophenyl)-2-oxo-1,4-diazaspiro[4.6]undec-3-en-1-yl]-N-(2-methylphenyl)acetamide |
| 93 | 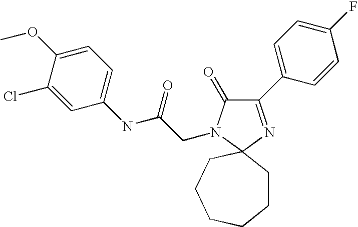 | N-[3-chloro-4-(methyloxy)phenyl]-2-[3-(4-fluorophenyl)-2-oxo-1,4-diazaspiro[4.6]undec-3-en-1-yl]acetamide |
| 94 | 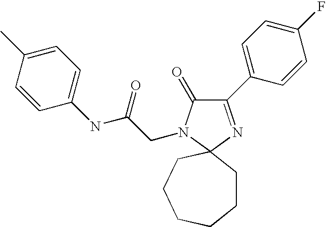 | 2-[3-(4-fluorophenyl)-2-oxo-1,4-diazaspiro[4.6]undec-3-en-1-yl]-N-(4-methylphenyl)acetamide |
| 95 | 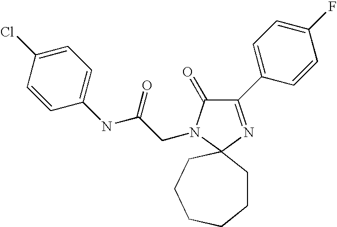 | N-(4-chlorophenyl)-2-[3-(4-fluorophenyl)-2-oxo-1,4-diazaspiro[4.6]undec-3-en-1-yl]acetamide |
| 96 | 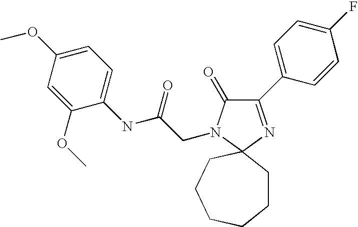 | N-[2,4-bis(methyloxy)phenyl]-2-[3-(4-fluorophenyl)-2-oxo-1,4-diazaspiro[4.6]undec-3-en-1-yl]acetamide |
| 97 | 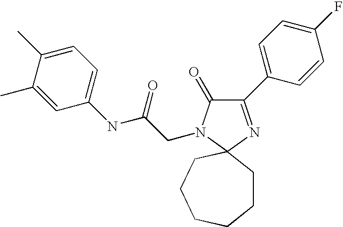 | N-(3,4-dimethylphenyl)-2-[3-(4-fluorophenyl)-2-oxo-1,4-diazaspiro[4.6]undec-3-en-1-yl]acetamide |
| 98 | 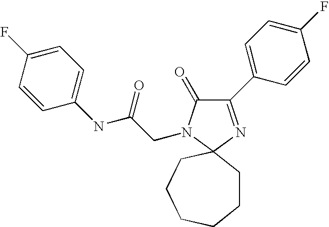 | N-(4-fluorophenyl)-2-[3-(4-fluorophenyl)-2-oxo-1,4-diazaspiro[4.6]undec-3-en-1-yl]acetamide |
| 99 | 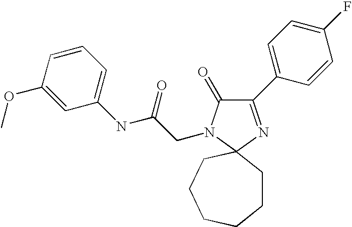 | 2-[3-(4-fluorophenyl)-2-oxo-1,4-diazaspiro[4.6]undec-3-en-1-yl]-N-[3-(methyloxy)phenyl]acetamide |
| 100 | 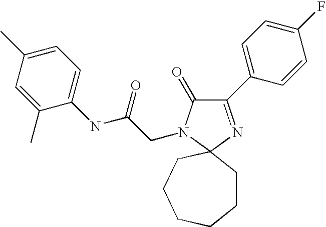 | N-(2,4-dimethylphenyl)-2-[3-(4-fluorophenyl)-2-oxo-1,4-diazaspiro[4.6]undec-3-en-1-yl]acetamide |
| 101 | 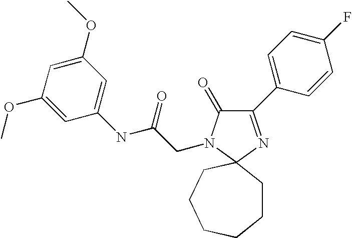 | N-[3,5-bis(methyloxy)phenyl]-2-[3-(4-fluorophenyl)-2-oxo-1,4-diazaspiro[4.6]undec-3-en-1-yl]acetamide |
| 102 | 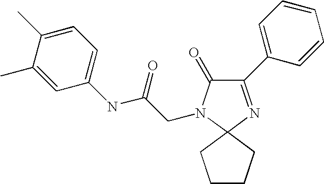 | N-(3,4-dimethylphenyl)-2-(2-oxo-3-phenyl-1,4-diazaspiro[4.4]non-3-en-1-yl)acetamide |
| 103 | 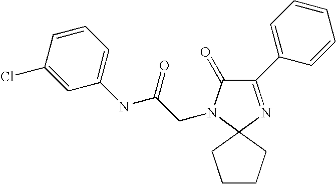 | N-(3-chlorophenyl)-2-(2-oxo-3-phenyl-1,4-diazaspiro[4.5]non-3-en-1-yl)acetamide |
| 104 | 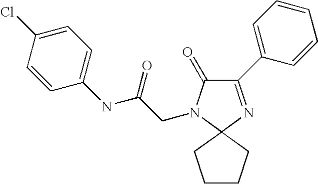 | N-(4-chlorophenyl)-2-(2-oxo-3-phenyl-1,4-diazaspiro[4.4]non-3-en-1-yl)acetamide |
| 105 | 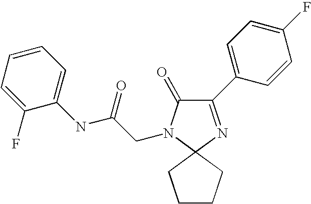 | N-(2-fluorophenyl)-2-[3-(4-fluorophenyl)-2-oxo-1,4-diazaspiro[4.4]non-3-en-1-yl]acetamide |
| 106 | 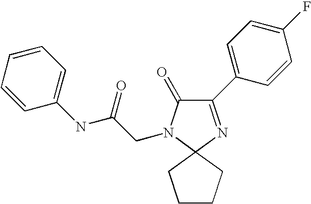 | 2-[3-(4-fluorophenyl)-2-oxo-1,4-diazaspiro[4.4]non-3-en-1-yl]-N-phenylacetamide |
| 107 | 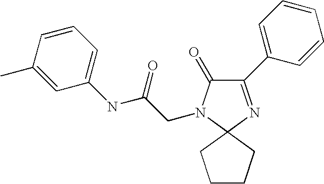 | N-(3-methylphenyl)-2-(2-oxo-3-phenyl-1,4-diazaspiro[4.4]non-3-en-1-yl)acetamide |
| 108 | 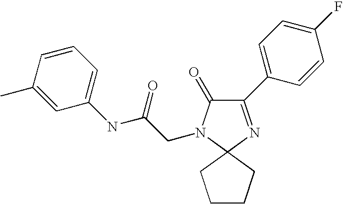 | 2-[3-(4-fluorophenyl)-2-oxo-1,4-diazaspiro[4.4]non-3-en-1-yl]-N-(3-methylphenyl)acetamide |
| 109 | 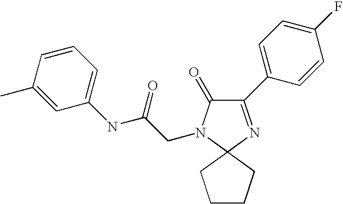 | N-(2,3-dimethylphenyl)-2-[3-(4-fluorophenyl)-2-oxo-1,4-diazaspiro[4.4]non-3-en-1-yl]acetamide |
| 110 | 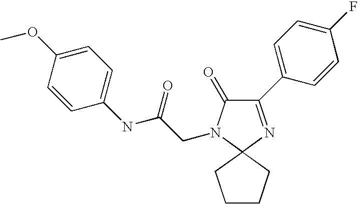 | 2-[3-(4-fluorophenyl)-2-oxo-1,4-diazaspiro[4.4]non-3-en-1-yl]-N-[4-(methyloxy)phenyl]acetamide |
| 111 | 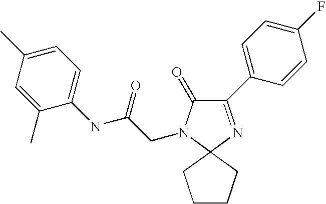 | N-(2,4-dimethyphenyl)-2-[3-(4-fluorophenyl)-2-oxo-1,4-diazaspiro[4.4]non-3-en-1-yl]acetamide |
| 112 | 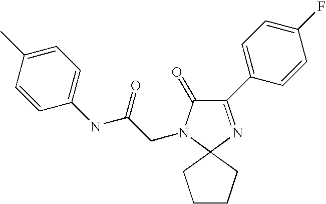 | 2-[3-(4-fluorophenyl)-2-oxo-1,4-diazaspiro[4.4]non-3-en-1-yl]-N-(4-methylphenyl)acetamide |
| 113 | 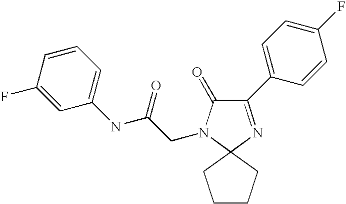 | N-(3-fluorophenyl)-2-[3-(4-fluorophenyl)-2-oxo-1,4-diazaspiro[4.4]non-3-en-1-yl]acetamide |
| 114 | 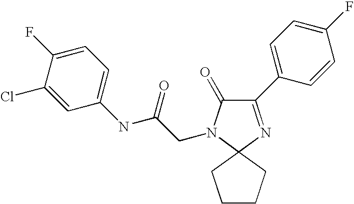 | N-(3-chloro-4-fluorophenyl)-2-[3-(4-fluorophenyl)-2-oxo-1,4-diazaspiro[4.4]non-3-en-1-yl]acetamide |
| 115 | 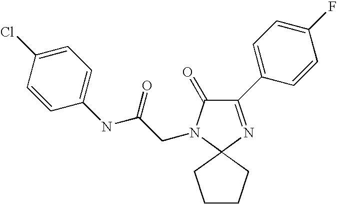 | N-(4-chlorophenyl)-2-[3-(4-fluorophenyl)-2-oxo-1,4-diazaspiro[4.4]non-3-en-1-yl]acetamide |
| 116 | 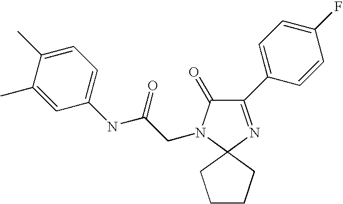 | N-(3,4-dimethylphenyl)-2-[3-(4-fluorophenyl)-2-oxo-1,4-diazaspiro[4.4]non-3-en-1-yl]acetamide |
| 117 | 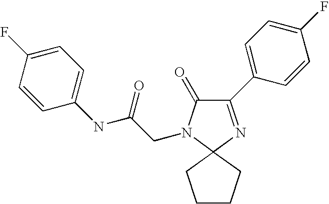 | N-(4-fluorophenyl)-2-[3-(4-fluorophenyl)-2-oxo-1,4-diazaspiro[4.4]non-3-en-1-yl]acetamide |
| 118 | 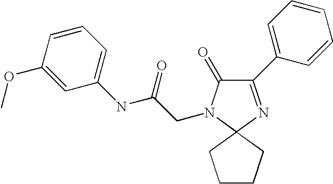 | N-[3-(methyloxy)phenyl]-2-(2-oxo-3-phenyl-1,4-diazaspiro[4.4]non-3-en-1-yl)acetamide |

### Example 3

### N-(3-chlorophenyl)-2-{3-[4-(methyloxy)phenyl]-2-oxo-1,4-diazaspiro [4.5]dec-3-en-1-yl}acetamide Alternative method

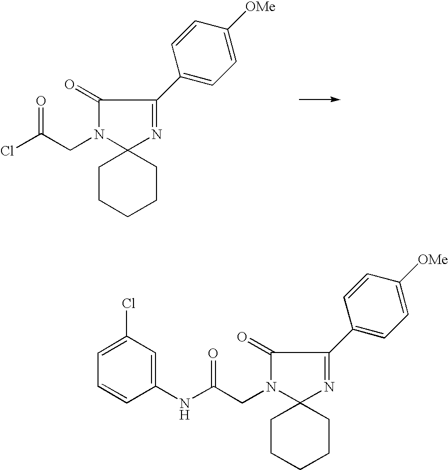

The title product (40 mg; 77%) was obtained from {3-[4-(methyloxy)phenyl]-2-oxo-1,4-diazaspiro[4.5]dec-3-en-1-yl}acetyl chloride D17 (60 mg; 0.18 mmol), 3-chloroaniline (25 mg; 0.2 mmol) and triethylamine (0.1 ml; 0.72 mmol) in DCM (4 ml) in a similar manner to that described in E5 alternative method, except that the product was purified using Mass Directed Auto-Purification System chromatography. ¹H NMR (CDCl₃) δ: 1.34-1.36 (3H, m), 1.82-1.91 (3H, m), 1.97-2.08 (4H, m), 3.89 (3H, s), 4.23 (2H, s), 6.98-7.01 (2H, m), 7.01-7.08 (1H, m), 7.19-7.23 (1H, m), 7.31-7.33 (1H, m), 7.62-7.63 (1H, m), 8.43-8.47 (2H, m), 9.02 (1H, s). Mass Spectrum (Electrospray LC/MS; MeOH): Found 426 (MH⁺). C₂₃H₂₄³⁵ClN₃O₃ requires 425. Ret. time 3.55 min.

### Example 5

### 2-[3-(4-Chlorophenyl)-2-oxo-1,4-diazaspiro[4.5]dec-3-en-1-yl]-N-(3,4-difluorophenyl)acetamide

Alternative Method

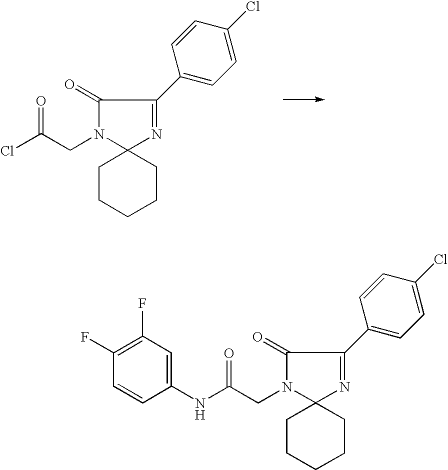

A mixture of [3-(4-chlorophenyl)-2-oxo-1,4-diazaspiro[4.5]dec-3-en-1-yl]acetyl chloride D11 (654 mg; 1.93 mmol), 3,4-difluoroaniline (258 mg; 2.00 mmol) and triethylamine (0.55 ml; 4.00 mmol) in DCM (50 ml) was stirred at room temperature overnight. Saturated sodium bicarbonate was added and stirred for 1 h. The organics were separated and dried using a phase-separation cartridge and evaporated to a white solid which was partitioned between 2NHCl and ethyl acetate. The organics were separated and dried using a phase-separation cartridge. Chromatography on silica gel (Biotage SP4) eluting with a 12-100% ethyl acetate-pentane gradient afforded the product which was then triturated with 40-60° C. petrol and filtered to afford the title product (263 mg; 32%). ¹H NMR (CDCl₃) δ: 1.30-1.37 (3H, m), 1.82-2.09 (7H, m), 4.22 (2H, s), 7.03-7.11 (2H, m), 7.42-7.48 (2H, m), 7.56-7.62 (1H, m), 8.41-8.46 (2H, m), 8.98 (1H, s). Mass Spectrum (Electrospray LC/MS; MeOH): Found 432 (MH⁺). C₂₂H₂₀³⁵ClF₂N₃O₂ requires 431. Ret. time 3.69 min.

### Example 17

### 2-[3-(4-Chlorophenyl)-2-oxo-1,4-diazaspiro[4.5]dec-3-en-1-yl]-N-(3-fluorophenyl)acetamide

Alternative Method

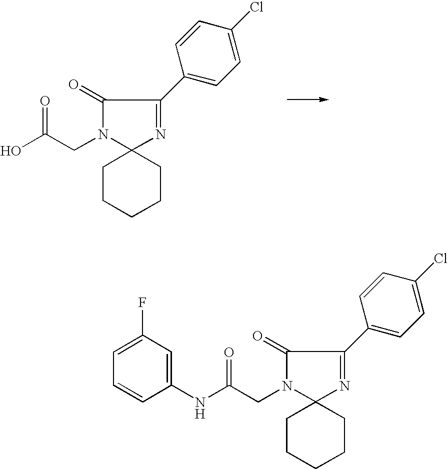

A mixture of [3-(4-chlorophenyl)-2-oxo-1,4-diazaspiro[4.5]dec-3-en-1-yl]acetic acid D10 (60 mg; 0.19 mol), DIPEA (0.1 ml; 0.57 mmol), HATU (76 mg; 0.2 mmol) and 3-fluoroaniline (22 mg; 0.19 mmol) in DCM (5 ml) was shaken overnight. The reaction mixture was washed with saturated sodium bicarbonate, the organics separated with a phase-separation cartridge and the organics chromatographed on silica gel (Biotage SP4) eluting with a 0-100% ethyl acetate-pentane gradient to afford a product which was then further purified by Mass Directed Auto-Purification System chromatography to afford the title product. ¹H NMR (CDCl₃) δ: 1.32-1.37 (3H, m), 1.82-2.09 (7H, m), 4.22 (2H, s), 7.02-7.08 (3H, m), 7.45-7.48 (2H, m), 7.56-7.61 (1H, m), 8.41-8.44 (2H, m), 8.99 (1H, s). Mass Spectrum (Electrospray LC/MS; MeOH): Found 414 (MH⁺). C₂₂H₂₁³⁵ClFN₃O₂ requires 413. Ret. time 3.72 min.

### Example 21

### 2-[3-(4-Chlorophenyl)-2-oxo-1,4-diazaspiro[4.5]dec-3-en-1-yl]-N-(2,4-dimethylphenyl)acetamide

Alternative Method

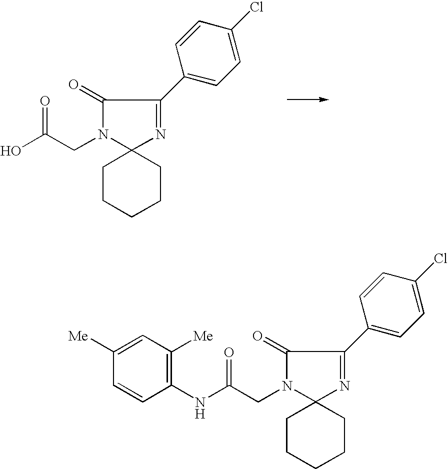

The title product was prepared from [3-(4-chlorophenyl)-2-oxo-1,4-diazaspiro[4.5]dec-3-en-1-yl]acetic acid D10 (60 mg; 0.19 mol), DIPEA (0.1 ml; 0.57 mmol), HATU (76 mg; 0.2 mmol) and 2,4-dimethylaniline (24 mg; 0.19 mmol) in DCM (5 ml) in a similar manner to that described in E17 alternative method with Mass Directed Auto-Purification System chromatography for work-up and purification. ¹H NMR (CDCl₃) δ: 1.35-1.41 (3H, m), 1.81-2.13 (7H, m), 2.19 (3H,s), 2.27 (3H, s), 4.26 (2H, s), 6.97-6.99 (2H, m), 7.44-7.47 (2H, m), 7.65-7.67 (1H, m), 8.42-8.45 (3H, m). Mass Spectrum (Electrospray LC/MS; MeOH): Found 424 (MH⁺). C₂₄H₂₆³⁵ClN₃O₂ requires 423. Ret. time 3.77 min.

### Example 24

### 2-[3-(4-Chlorophenyl)-2-oxo-1,4-diazaspiro[4.5]dec-3-en-1-yl]-N-(3,4-dimethylphenyl)acetamide

Alternative Method

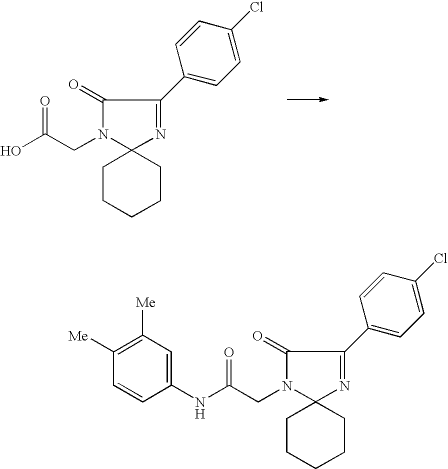

The title product was prepared from [3-(4-chlorophenyl)-2-oxo-1,4-diazaspiro[4.5]dec-3-en-1-yl]acetic acid D10 (60 mg; 0.19 mol), DIPEA (0.1 ml; 0.57 mmol), HATU (76 mg; 0.2 mmol) and 3,4-dimethylaniline (24 mg; 0.19 mmol) in DCM (5 ml) in a similar manner to that described in E17 alternative method with Mass Directed Auto-Purification System chromatography for work-up and purification. ¹H NMR (CDCl₃) δ: 1.34-1.40 (3H, m), 1.80-2.11 (7H, m), 2.20 (3H,s), 2.22 (3H, s), 4.22 (2H, s), 7.04-7.06 (1H, m), 7.20-7.23 (2H, m), 7.45-7.48 (2H, m), 8.42-8.45 (2H, m), 8.56 (1H, s). Mass Spectrum (Electrospray LC/MS; MeOH): Found 424 (MH⁺). C₂₄H₂₆³⁵ClN₃O₂ requires 423. Ret. time 3.82 min.

### Example 46

### 2-(2-oxo-3-phenyl-1,4-diazaspiro[4.5]dec-3-en-1-yl)-N-phenyl acetamide

Alternative Method

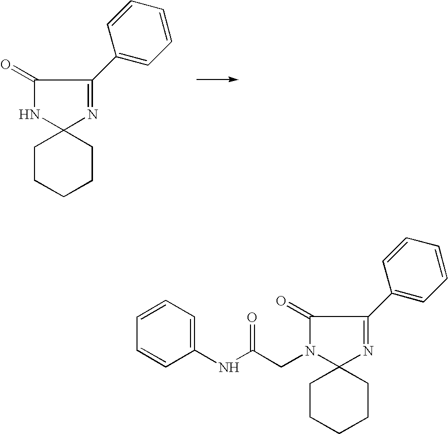

To a solution of 3-phenyl-1,4-diazaspiro[4.5]dec-3-en-2-one D19 (100 mg; 0.44 mmol) in DMF (3 ml) was added 60% sodium hydride dispersed in oil (18 mg, 0.44 mmol) and the mixture shaken for 30 min. 2-Chloro-N-phenylacetamide (75 mg; 0.44 mmol) was added, the mixture shaken overnight at room temperature and then partitioned between water and DCM. The organics were washed with water (×4), dried with a phase-separation cartridge and evaporated under reduced pressure to a yellow oil which was chromatographed on silica gel (Biotage Horizon), eluting with 0-100% ethyl acetate-pentane gradient. The product was then further purified with Mass Directed Auto-Purification System chromatography to afford the title product (47 mg; 30%). ¹H NMR (CDCl₃) δ: 1.36-1.39 (3H, m), 1.81-2.12 (7H, m), 4.25 (2H, s), 7.08-7.11 (1H, m), 7.26-7.32 (3H, m), 7.49-7.56 (4H, m), 8.45-8.48 (2H, m), 8.83 (1H, s). Mass Spectrum (Electrospray LC/MS; MeOH): Found 362 (MH⁺). C₂₂H₂₃N₃O₂ requires 361. Ret. time 3.33 min.

Method A

### Example 119

### N-(4-Fluoro-3-methylphenyl)-2-{3-[4-(methyloxy)phenyl]-2-oxo-1,4-diazaspiro[4.5]dec-3-en-1-yl}acetamide

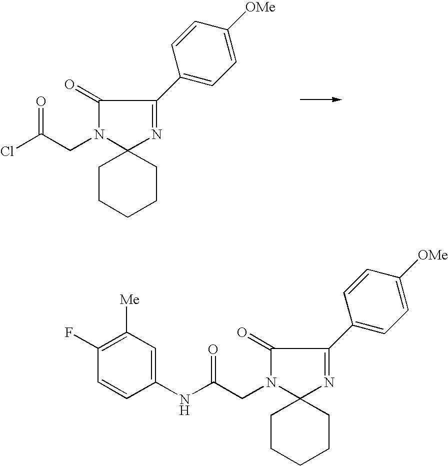

A mixture of {3-[4-(methyloxy)phenyl]-2-oxo-1,4-diazaspiro[4.5]dec-3-en-1-yl}acetyl chloride D17 (150 mg; 0.46 mmol), 4-fluoro-3-methylaniline (57 mg; 0.46 mmol) and triethylamine (0.13 ml; 0.46 mmol) in DCM (5 ml) was stirred at room temperature overnight. The reaction was washed with water and the organics separated, dried, and chromatographed on silica gel eluting with a 0-50% ethyl acetate-pentane gradient to afford the product (100 mg; 53%). ¹H NMR (CDCl₃) δ: 1.34-1.36 (3H, m), 1.80-2.08 (7H, m), 2.23 (3H, s), 3.88 (3H, s), 4.22 (2H, s), 6.89-6.94 (1H, m), 6.98-7.01 (2H, m), 7.23-7.32 (2H, m), 8.43-8.47 (2H, m), 8.78 (1H, s). Mass Spectrum (Electrospray LC/MS; MeOH): Found 424 (MH⁺). C₂₄H₂₆FN₃O₃ requires 423. Ret. time 3.40 min.

Method B

### Example 120

### N-[2-(Methyloxy)phenyl]-2-[3-(4-methylphenyl)-2-oxo-1,4-diazaspiro[4.5]dec-3-en-1-yl]acetamide

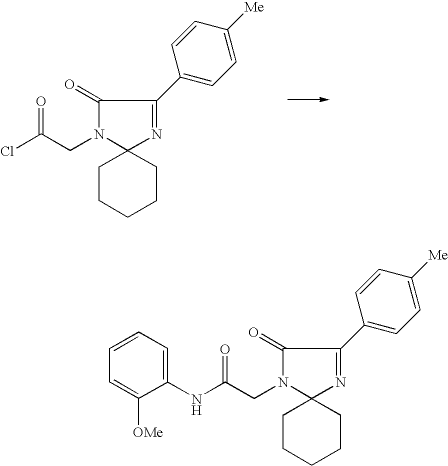

The title product was obtained from [3-(4-methylphenyl)-2-oxo-1,4-diazaspiro[4.5]dec-3-en-1-yl]acetyl chloride D25 (80 mg; 0.25 mmol), 2-methoxyaniline (46 mg; 0.37 mmol) and triethylamine (75 mg; 0.75 mmol) in DCM (10 ml) using the method of E119. After chromatography on silica gel the product (Mass Spectrum (Electrospray LC/MS; MeOH): Found 406 (MH⁺). C₂₄H₂₇N₃O₃ requires 405. Ret. time 3.68 min.) was further purified using a scavenger resin. ¹H NMR (CDCl₃) inter alia δ: 1.79-2.06 (7H, m), 2.43 (3H, s), 3.79 (3H, s), 4.26 (2H, s), 6.82-6.84 (1H, m), 6.91-6.94 (1H, m), 7.02-7.06 (1H, m), 7.26-7.30 (2H, m), 8.23-8.25 (1H, m), 8.39-8.41 (2H, m), 8.69 (1H, s).

Method C

### Example 121

### 2-[3-(4-Chlorophenyl)-2-oxo-1,4-diazaspiro[4.5]dec-3-en-1-yl]-N-(2,3-difluorophenyl)acetamide

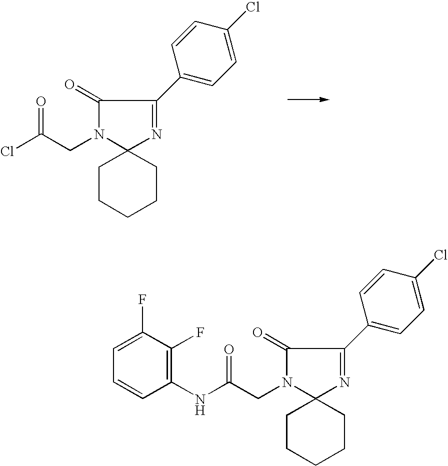

To a solution of [3-(4-chlorophenyl)-2-oxo-1,4-diazaspiro[4.5]dec-3-en-1-yl]acetyl chloride D11 (105 mg; 0.313 mmol) and triethylamine (0.09 ml; 0.625 mmol) in DCM (3 ml) was added a solution of 2,3-difluoroaniline (40 mg; 0.313 mmol) in DCM (1 ml). The resultant solution was allowed to stand at room temperature overnight and then washed with saturated aqueous sodium bicarbonate (8 ml). The organic layer was passed through a phase separation cartridge and the solvent removed under reduced pressure. The residue was dissolved in DMSO and purified using Mass Directed Auto-Purification System chromatography to afford the title product (45 mg; 33%). ¹H NMR (CDCl₃) δ: 1.29-1.42 (3H, m), 1.84-2.08 (7H, m), 4.28 (2H, s), 6.87-6.94 (1H, m), 7.01-7.01 (1H, m), 7.45-7.48 (2H, m), 7.92-7.96 (1H, s), 8.43-8.46 (2H, m), 8.92 (1H, s). Mass Spectrum (Electrospray LC/MS; MeOH): Found 432 (MH⁺). C₂₂H₂₀³⁵ClF₂N₃O₂ requires 431. Ret. time 3.63 min.

The examples in Table 2 were prepared from the appropriate 3-(substitutedphenyl)-1,4-diazaspiro[4.5]dec-3-en-2-one (which was synthesized using methods analogous to those described in D5 method 2) using methods similar to Example 119 Method A, Example 120 Method B and Example 121 Method C.

TABLE 2

| | | | Mass spectrum | |
|---|---|---|---|---|
| | | | (Electrospray | |
| | | | LC/MS), API⁺ | |
| Example | Structure | Method | Ret. time (min) | Name |
| 122 | 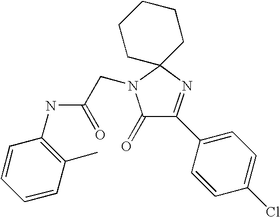 | A | Found 410 (MH⁺)C₂₃H₂₄³⁵ClN₃O₂requires 409;3.69 | 2-[3-(4-chlorophenyl)-2-oxo-1,4-diazaspiro[4.5]dec-3-en-1-yl]-N-(2-methylphenyl)acetamide |
| 123 | 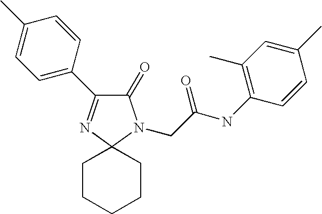 | B | Found 404 (MH⁺)C₂₅H₂₉N₃O₂requires 403;3.70 | N-(2,4-dimethylphenyl)-2-[3-(4-methylphenyl)-2-oxo-1,4-diazaspiro[4.5]dec-3-en-1-yl]acetamide |
| 124 | 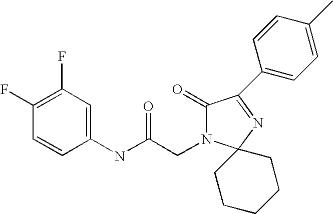 | B | Found 412 (MH⁺)C₂₃H₂₃F₂N₃O₂requires 411;3.66 | N-(3,4-difluorophenyl)-2-[3-(4-methylphenyl)-2-oxo-1,4-diazaspiro[4.5]dec-3-en-1-yl]acetamide |
| 125 | 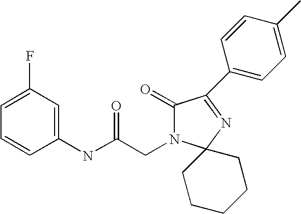 | B | Found 394 (MH⁺)C₂₃H₂₄FN₃O₂requires 393;3.62 | N-(3-fluorophenyl)-2-[3-(4-methylphenyl)-2-oxo-1,4-diazaspiro[4.5]dec-3-en-1-yl]acetamide |
| 126 | 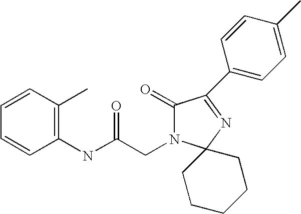 | B | Found 390 (MH⁺)C₂₄H₂₇N₃O₂requires 389;3.58 | N-(2-methylphenyl)-2-[3-(4-methylphenyl)-2-oxo-1,4-diazaspiro[4.5]dec-3-en-1-yl]acetamide |
| 127 | 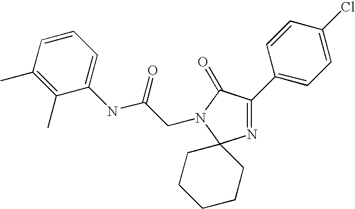 | A | Found 424 (MH⁺)C₂₄H₂₆³⁵ClN₃O₂requires 423;3.58 | 2-[3-(4-chlorophenyl)-2-oxo-1,4-diazaspiro[4.5]dec-3-en-1-yl]-N-(2,3-dimethylphenyl)acetamide |
| 128 | 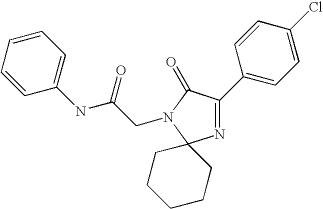 | A | Found 396 (MH⁺)C₂₂H₂₂³⁵ClN₃O₂requires 395;3.45 | 2-[3-(4-chlorophenyl)-2-oxo-1,4-diazaspiro[4.5]dec-3-en-1-yl]-N-phenylacetamide |
| 129 | 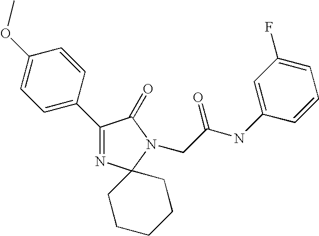 | C | Found 410 (MH⁺)C₂₃H₂₄FN₃O₃requires 409;3.40 | N-(3-fluorophenyl)-2-{3-[4-(methyloxy)phenyl]-2-oxo-1,4-diazaspiro[4.5]dec-3-en-1-yl}acetamide |
| 130 | 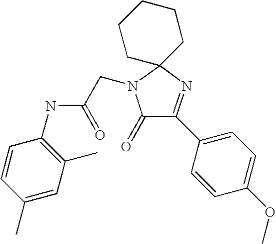 | C | Found 420 (MH⁺)C₂₅H₂₉N₃O₃requires 419;3.48 | N-(2,4-dimethylphenyl)-2-{3-[4-(methyloxy)phenyl]-2-oxo-1,4-diazaspiro[4.5]dec-3-en-1-yl}acetamide |
| 131 | 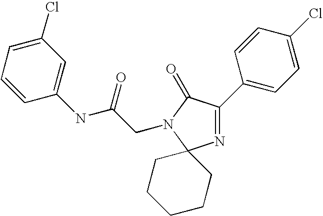 | A | Found 430 (MH⁺)C₂₂H₂₁³⁵Cl₂N₃O₂requires 429;3.74 | N-(3-chlorophenyl)-2-[3-(4-chlorophenyl)-2-oxo-1,4-diazaspiro[4.5]dec-3-en-1-yl]acetamide |
| 132 | 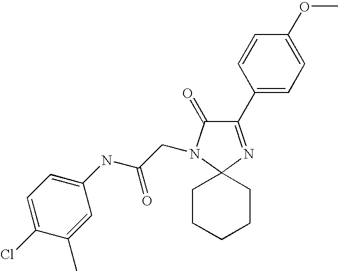 | A | Found 440 (MH⁺)C₂₄H₂₆³⁵ClN₃O₃requires 439;3.58 | N-(4-chloro-3-methylphenyl)-2-{3-[4-(methyloxy)phenyl]-2-oxo-1,4-diazaspiro[4.5]dec-3-en-1-yl}acetamide |
| 133 | 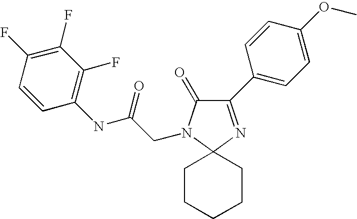 | A | Found 446 (MH⁺)C₂₃H₂₂F₃N₃O₃requires 445;3.42 | 2-{3-[4-(methyloxy)phenyl]-2-oxo-1,4-diazaspiro[4.5]dec-3-en-1-yl}-N-(2,3,4-trifluorophenyl)acetamide |

1-14. (canceled)

15. A compound of formula (I) or a salt thereof 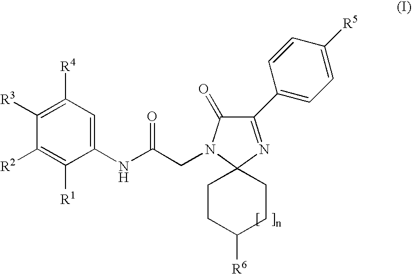
wherein:
R¹ is H, methyl, methoxy or fluoro;
R² is H, methyl, methoxy, fluoro or chloro;
R³ is H, methyl, methoxy, fluoro or chloro;
R⁴ is H or methoxy;
R⁵ is H, methyl, methoxy, chloro or fluoro;
R⁶ is H or methyl; and
n is 0, 1 and 2;
excluding:
N-[3,4-bis(methyloxy)phenyl]-2-(8-methyl-2-oxo-3-phenyl-1,4-diazaspiro[4.5]dec-3-en-1-yl)acetamide,
2-(8-methyl-2-oxo-3-phenyl-1,4-diazaspiro[4.5]dec-3-en-1-yl)-N-[4-(methyloxy)phenyl]acetamide,
2-[3-(4-fluorophenyl)-2-oxo-1,4-diazaspiro[4.4]non-3-en-1-yl]-N-(2-methylphenyl)acetamide,
2-(2-oxo-3-phenyl-1,4-diazaspiro[4.4]non-3-en-1-yl)-N-phenylacetamide,
N-(4-methylphenyl)-2-(2-oxo-3-phenyl-1,4-diazaspiro[4.4]non-3-en-1-yl)acetamide,
N-[4-(methyloxy)phenyl]-2-(2-oxo-3-phenyl-1,4-diazaspiro[4.4]non-3-en-1-yl)acetamide,
N-[3,4-bis(methyloxy)phenyl]-2-(2-oxo-3-phenyl-1,4-diazaspiro[4.4]non-3-en-1-yl)acetamide, and
N-[3,5-bis(methyloxy)phenyl]-2-(2-oxo-3-phenyl-1,4-diazaspiro[4.4]non-3-en-1-yl)acetamide.

16. A compound as claimed in claim 15, wherein R¹ is H, methyl or methoxy and R³ is H, methyl, fluoro or chloro; or a salt thereof.

17. A compound as claimed in claim 15 wherein R¹ is H, methyl or methoxy; R² is H, methyl, methoxy or fluoro; R³ is H, methyl, or fluoro; and n is 1 or 2, or a salt thereof.

18. A compound as claim 15 wherein R⁵ is methoxy, chloro, methyl or fluoro; or a salt thereof.

19. A compound as claimed in claim 15 wherein R⁵ is methoxy or chloro, or a salt thereof.

20. A compound as claimed in claim 15 which is selected from the group consisting of:
2-[3-(4-chlorophenyl)-2-oxo-1,4-diazaspiro[4.5]dec-3-en-1-yl]-N-[2-(methyloxy)phenyl]acetamide
2-[3-(4-chlorophenyl)-2-oxo-1,4-diazaspiro[4.5]dec-3-en-1-yl]-N-(3-methylphenyl)acetamide;
N-(3-chlorophenyl)-2-{3-[4-(methyloxy)phenyl]-2-oxo-1,4-diazaspiro[4.5]dec-3-en-1-yl}acetamide;
N-(3-methylphenyl)-2-[3-(4-methylphenyl)-2-oxo-1,4-diazaspiro[4.5]dec-3-en-1-yl]acetamide;
2-[3-(4-chlorophenyl)-2-oxo-1,4-diazaspiro[4.5]dec-3-en-1-yl]-N-(3,4-difluorophenyl)acetamide;
N-[3,5-bis(methyloxy)phenyl]-2-[3-(4-chlorophenyl)-2-oxo-1,4-diazaspiro[4.5]dec-3-en-1-yl]acetamide;
2-{3-[4-(methyloxy)phenyl]-2-oxo-1,4-diazaspiro[4.6]undec-3-en-1-yl}-N-phenylacetamide;
N-(3-chloro-4-fluorophenyl)-2-{3-[4-(methyloxy)phenyl]-2-oxo-1,4-diazaspiro[4.6]undec-3-en-1-yl}acetamide;
N-(3-chloro-4-fluorophenyl)-2-(2-oxo-3-phenyl-1,4-diazaspiro[4.6]undec-3-en-1-yl)acetamide;
N-(3-chloro-4-fluorophenyl)-2-{3-[4-(methyloxy)phenyl]-2-oxo-1,4-diazaspiro[4.5]dec-3-en-1-yl}acetamide;
N-(3-chloro-4-fluorophenyl)-2-(2-oxo-3-phenyl-1,4-diazaspiro[4.5]dec-3-en-1-yl)acetamide;
N-[3-(methyloxy)phenyl]-2-[3-(4-methylphenyl)-2-oxo-1,4-diazaspiro[4.5]dec-3-en-1-yl]acetamide;
2-{3-[4-(methyloxy)phenyl]-2-oxo-1,4-diazaspiro[4.6]undec-3-en-1-yl}-N-(3-methylphenyl)acetamide;
N-(3,4-difluorophenyl)-2-{3-[4-(methyloxy)phenyl]-2-oxo-1,4-diazaspiro[4.5]dec-3-en-1-yl}acetamide;
N-[3-(methyloxy)phenyl]-2-[3-(4-methylphenyl)-2-oxo-1,4-diazaspiro[4.6]undec-3-en-1-yl]acetamide;
N-(3-chloro-4-fluorophenyl)-2-(8-methyl-2-oxo-3-phenyl-1,4-diazaspiro[4.5]dec-3-en-1-yl)acetamide;
2-[3-(4-chlorophenyl)-2-oxo-1,4-diazaspiro[4.5]dec-3-en-1-yl]-N-(3-fluorophenyl)acetamide;
N-(3-chlorophenyl)-2-(2-oxo-3-phenyl-1,4-diazaspiro[4.5]dec-3-en-1-yl)acetamide;
N-(3,4-dimethylphenyl)-2-[3-(4-methylphenyl)-2-oxo-1,4-diazaspiro[4.6]undec-3-en-1-yl]acetamide;
N-(4-fluorophenyl)-2-[3-(4-methylphenyl)-2-oxo-1,4-diazaspiro[4.6]undec-3-en-1-yl]acetamide;
2-[3-(4-chlorophenyl)-2-oxo-1,4-diazaspiro[4.5]dec-3-en-1-yl]-N-(2,4-dimethylphenyl)acetamide;
2-[3-(4-chlorophenyl)-2-oxo-1,4-diazaspiro[4.5]dec-3-en-1-yl]-N-(4-fluorophenyl)acetamide;
N-(3-methylphenyl)-2-[3-(4-methylphenyl)-2-oxo-1,4-diazaspiro[4.6]undec-3-en-1-yl]acetamide;
2-[3-(4-chlorophenyl)-2-oxo-1,4-diazaspiro[4.5]dec-3-en-1-yl]-N-(3,4-dimethylphenyl)acetamide;
N-(3-methylphenyl)-2-(2-oxo-3-phenyl-1,4-diazaspiro[4.6]undec-3-en-1-yl)acetamide;
N-(2,4-dimethylphenyl)-2-{3-[4-(methyloxy)phenyl]-2-oxo-1,4-diazaspiro[4.6]undec-3-en-1-yl}acetamide;
N-(3,4-dimethylphenyl)-2-{3-[4-(methyloxy)phenyl]-2-oxo-1,4-diazaspiro[4.5]dec-3-en-1-yl}acetamide;
N-(3,4-dimethylphenyl)-2-{3-[4-(methyloxy)phenyl]-2-oxo-1,4-diazaspiro[4.6]undec-3-en-1-yl}acetamide;
2-{3-[4-(methyloxy)phenyl]-2-oxo-1,4-diazaspiro[4.5]dec-3-en-1-yl}-N-(3-methylphenyl)acetamide;
N-(3-chlorophenyl)-2-(8-methyl-2-oxo-3-phenyl-1,4-diazaspiro[4.5]dec-3-en-1-yl)acetamide;
2-[3-(4-fluorophenyl)-2-oxo-1,4-diazaspiro[4.5]dec-3-en-1-yl]-N-(3-methylphenyl)acetamide;
N-(3-chlorophenyl)-2-[3-(4-fluorophenyl)-2-oxo-1,4-diazaspiro[4.6]undec-3-en-1-yl]acetamide;
2-[3-(4-fluorophenyl)-2-oxo-1,4-diazaspiro[4.6]undec-3-en-1-yl]-N-(3-methylphenyl)acetamide;
N-(3-chloro-4-fluorophenyl)-2-[3-(4-fluorophenyl)-2-oxo-1,4-diazaspiro[4.5]dec-3-en-1-yl]acetamide;
2-(8-methyl-2-oxo-3-phenyl-1,4-diazaspiro[4.5]dec-3-en-1-yl)-N-phenylacetamide;
N-(3-methylphenyl)-2-(2-oxo-3-phenyl-1,4-diazaspiro[4.5]dec-3-en-1-yl)acetamide;
2-(8-methyl-2-oxo-3-phenyl-1,4-diazaspiro[4.5]dec-3-en-1-yl)-N-(3-methylphenyl)acetamide;
N-(4-methylphenyl)-2-[3-(4-methylphenyl)-2-oxo-1,4-diazaspiro[4.5]dec-3-en-1-yl]acetamide;
2-{3-[4-(methyloxy)phenyl]-2-oxo-1,4-diazaspiro[4.5]dec-3-en-1-yl}-N-(4-methylphenyl)acetamide;
N-(3,4-dimethylphenyl)-2-(2-oxo-3-phenyl-1,4-diazaspiro[4.5]dec-3-en-1-yl)acetamide;
N-(4-chlorophenyl)-2-(2-oxo-3-phenyl-1,4-diazaspiro[4.5]dec-3-en-1-yl)acetamide;
N-(4-fluorophenyl)-2-(2-oxo-3-phenyl-1,4-diazaspiro[4.5]dec-3-en-1-yl)acetamide;
N-(3,4-difluorophenyl)-2-(2-oxo-3-phenyl-1,4-diazaspiro[4.5]dec-3-en-1-yl)acetamide;
2-[3-(4-chlorophenyl)-2-oxo-1,4-diazaspiro[4.5]dec-3-en-1-yl]-N-(2,4-difluorophenyl)acetamide;
N-[3,5-bis(methyloxy)phenyl]-2-(8-methyl-2-oxo-3-phenyl-1,4-diazaspiro[4.5]dec-3-en-1-yl)acetamide;
2-(2-oxo-3-phenyl-1,4-diazaspiro[4.5]dec-3-en-1-yl)-N-phenylacetamide;
2-[3-(4-fluorophenyl)-2-oxo-1,4-diazaspiro[4.5]dec-3-en-1-yl]-N-(2-methylphenyl)acetamide;
N-(2,3-dimethylphenyl)-2-[3-(4-fluorophenyl)-2-oxo-1,4-diazaspiro[4.5]dec-3-en-1-yl]acetamide;
N-(4-chlorophenyl)-2-{3-[4-(methyloxy)phenyl]-2-oxo-1,4-diazaspiro[4.5]dec-3-en-1-yl}acetamide;
N-(4-fluorophenyl)-2-{3-[4-(methyloxy)phenyl]-2-oxo-1,4-diazaspiro[4.5]dec-3-en-1-yl}acetamide;
N-(3,4-difluorophenyl)-2-(8-methyl-2-oxo-3-phenyl-1,4-diazaspiro[4.5]dec-3-en-1-yl)acetamide;
N-[3,5-bis(methyloxy)phenyl]-2-{3-[4-(methyloxy)phenyl]-2-oxo-1,4-diazaspiro[4.5]dec-3-en-1-yl}acetamide;
N-(2,4-dimethylphenyl)-2-[3-(4-fluorophenyl)-2-oxo-1,4-diazaspiro[4.5]dec-3-en-1-yl]acetamide;
N-[3-(methyloxy)phenyl]-2-{3-[4-(methyloxy)phenyl]-2-oxo-1,4-diazaspiro[4.5]dec-3-en-1-yl}acetamide;
2-[3-(4-fluorophenyl)-2-oxo-1,4-diazaspiro[4.5]dec-3-en-1-yl]-N-[3-(methoxyphenyl]acetamide;
N-[3,5-bis(methyloxy)phenyl]-2-[3-(4-methylphenyl)-2-oxo-1,4-diazaspiro[4.5]dec-3-en-1-yl]acetamide;
N-(4-fluorophenyl)-2-[3-(4-fluorophenyl)-2-oxo-1,4-diazaspiro[4.5]dec-3-en-1-yl]acetamide;
N-[4-(methyloxy)phenyl]-2-[3-(4-methylphenyl)-2-oxo-1,4-diazaspiro[4.5]dec-3-en-1-yl]acetamide;
N-(4-fluorophenyl)-2-(8-methyl-2-oxo-3-phenyl-1,4-diazaspiro[4.5]dec-3-en-1-yl)acetamide;
2-[3-(4-chlorophenyl)-2-oxo-1,4-diazaspiro[4.5]dec-3-en-1-yl]-N-[4-(methyloxy)phenyl]acetamide;
N-[2,4-bis(methyloxy)phenyl]-2-[3-(4-fluorophenyl)-2-oxo-1,4-diazaspiro[4.5]dec-3-en-1-yl]acetamide;
N-(4-methylphenyl)-2-(2-oxo-3-phenyl-1,4-diazaspiro[4.5]dec-3-en-1-yl)acetamide;
N-(4-chlorophenyl)-2-[3-(4-fluorophenyl)-2-oxo-1,4-diazaspiro[4.5]dec-3-en-1-yl]acetamide;
2-[3-(4-fluorophenyl)-2-oxo-1,4-diazaspiro[4.5]dec-3-en-1-yl]-N-[2-(methyloxy)phenyl]acetamide;
N-(3,4-dimethylphenyl)-2-[3-(4-fluorophenyl)-2-oxo-1,4-diazaspiro[4.5]dec-3-en-1-yl]acetamide;
N-[3-chloro-4-(methyloxy)phenyl]-2-(8-methyl-2-oxo-3-phenyl-1,4-diazaspiro[4.5]dec-3-en-1-yl)acetamide;
2-{3-[4-(methyloxy)phenyl]-2-oxo-1,4-diazaspiro[4.5]dec-3-en-1-yl}-N-phenylacetamide;
N-[3-(methyloxy)phenyl]-2-(2-oxo-3-phenyl-1,4-diazaspiro[4.5]dec-3-en-1-yl)acetamide;
2-(2-oxo-3-phenyl-1,4-diazaspiro[4.6]undec-3-en-1-yl)-N-phenylacetamide;
N-(4-methylphenyl)-2-(2-oxo-3-phenyl-1,4-diazaspiro[4.6]undec-3-en-1-yl)acetamide;
2-{3-[4-(methyloxy)phenyl]-2-oxo-1,4-diazaspiro[4.6]undec-3-en-1-yl}-N-(4-methylphenyl)acetamide;
N-(3,4-dimethylphenyl)-2-(2-oxo-3-phenyl-1,4-diazaspiro[4.6]undec-3-en-1-yl)acetamide;
N-(4-chlorophenyl)-2-(2-oxo-3-phenyl-1,4-diazaspiro[4.6]undec-3-en-1-yl)acetamide;
N-(4-chlorophenyl)-2-{3-[4-(methyloxy)phenyl]-2-oxo-1,4-diazaspiro[4.6]undec-3-en-1-yl}acetamide;
N-(4-fluorophenyl)-2-(2-oxo-3-phenyl-1,4-diazaspiro[4.6]undec-3-en-1-yl)acetamide;
N-(3-fluorophenyl)-2-(2-oxo-3-phenyl-1,4-diazaspiro[4.6]undec-3-en-1-yl)acetamide;
N-(3,4-difluorophenyl)-2-(2-oxo-3-phenyl-1,4-diazaspiro[4.6]undec-3-en-1-yl)acetamide;
N-[4-(methyloxy)phenyl]-2-(2-oxo-3-phenyl-1,4-diazaspiro[4.6]undec-3-en-1-yl)acetamide;
N-[3,4-bis(methyloxy)phenyl]-2-(2-oxo-3-phenyl-1,4-diazaspiro[4.6]undec-3-en-1-yl)acetamide;
N-[3-chloro-4-(methyloxy)phenyl]-2-(2-oxo-3-phenyl-1,4-diazaspiro[4.6]undec-3-en-1-yl)acetamide;
N-[3-(methyloxy)phenyl]-2-(2-oxo-3-phenyl-1,4-diazaspiro[4.6]undec-3-en-1-yl)acetamide;
N-[3,5-bis(methyloxy)phenyl]-2-(2-oxo-3-phenyl-1,4-diazaspiro[4.6]undec-3-en-1-yl)acetamide;
N-[3,5-bis(methyloxy)phenyl]-2-[3-(4-methylphenyl)-2-oxo-1,4-diazaspiro[4.6]undec-3-en-1-yl]acetamide;
N-[3-chloro-4-(methyloxy)phenyl]-2-[3-(4-methylphenyl)-2-oxo-1,4-diazaspiro[4.6]undec-3-en-1-yl]acetamide;
N-(4-chlorophenyl)-2-[3-(4-methylphenyl)-2-oxo-1,4-diazaspiro[4.6]undec-3-en-1-yl]acetamide;
N-(3-chloro-4-fluorophenyl)-2-[3-(4-methylphenyl)-2-oxo-1,4-diazaspiro[4.6]undec-3-en-1-yl]acetamide;
N-(3-chlorophenyl)-2-{3-[4-(methyloxy)phenyl]-2-oxo-1,4-diazaspiro[4.6]undec-3-en-1-yl}acetamide;
N-(2,3-dimethylphenyl)-2-[3-(4-fluorophenyl)-2-oxo-1,4-diazaspiro[4.6]undec-3-en-1-yl]acetamide;
N-(3-chloro-4-fluorophenyl)-2-[3-(4-fluorophenyl)-2-oxo-1,4-diazaspiro[4.6]undec-3-en-1-yl]acetamide;
2-[3-(4-fluorophenyl)-2-oxo-1,4-diazaspiro[4.6]undec-3-en-1-yl]-N-[2-(methyloxy)phenyl]acetamide;
N-(4-methylphenyl)-2-[3-(4-methylphenyl)-2-oxo-1,4-diazaspiro[4.6]undec-3-en-1-yl]acetamide;
2-[3-(4-fluorophenyl)-2-oxo-1,4-diazaspiro[4.6]undec-3-en-1-yl]-N-(2-methylphenyl)acetamide;
N-[3-chloro-4-(methyloxy)phenyl]-2-[3-(4-fluorophenyl)-2-oxo-1,4-diazaspiro[4.6]undec-3-en-1-yl]acetamide;
2-[3-(4-fluorophenyl)-2-oxo-1,4-diazaspiro[4.6]undec-3-en-1-yl]-N-(4-methylphenyl)acetamide;
N-(4-chlorophenyl)-2-[3-(4-fluorophenyl)-2-oxo-1,4-diazaspiro[4.6]undec-3-en-1-yl]acetamide;
N-[2,4-bis(methyloxy)phenyl]-2-[3-(4-fluorophenyl)-2-oxo-1,4-diazaspiro[4.6]undec-3-en-1-yl]acetamide;
N-(3,4-dimethylphenyl)-2-[3-(4-fluorophenyl)-2-oxo-1,4-diazaspiro[4.6]undec-3-en-1-yl]acetamide;
N-(4-fluorophenyl)-2-[3-(4-fluorophenyl)-2-oxo-1,4-diazaspiro[4.6]undec-3-en-1-yl]acetamide;
2-[3-(4-fluorophenyl)-2-oxo-1,4-diazaspiro[4.6]undec-3-en-1-yl]-N-[3-(methyloxy)phenyl]acetamide;
N-(2,4-dimethylphenyl)-2-[3-(4-fluorophenyl)-2-oxo-1,4-diazaspiro[4.6]undec-3-en-1-yl]acetamide
N-[3,5-bis(methyloxy)phenyl]-2-[3-(4-fluorophenyl)-2-oxo-1,4-diazaspiro[4.6]undec-3-en-1-yl]acetamide;
N-(3,4-dimethylphenyl)-2-(2-oxo-3-phenyl-1,4-diazaspiro[4.4]non-3-en-1-yl)acetamide;
N-(3-chlorophenyl)-2-(2-oxo-3-phenyl-1,4-diazaspiro[4.4]non-3-en-1-yl)acetamide;
N-(4-chlorophenyl)-2-(2-oxo-3-phenyl-1,4-diazaspiro[4.4]non-3-en-1-yl)acetamide;
N-(2-fluorophenyl)-2-[3-(4-fluorophenyl)-2-oxo-1,4-diazaspiro[4.4]non-3-en-1-yl]acetamide;
2-[3-(4-fluorophenyl)-2-oxo-1,4-diazaspiro[4.4]non-3-en-1-yl]-N-phenylacetamide;
N-(3-methylphenyl)-2-(2-oxo-3-phenyl-1,4-diazaspiro[4.4]non-3-en-1-yl)acetamide;
2-[3-(4-fluorophenyl)-2-oxo-1,4-diazaspiro[4.4]non-3-en-1-yl]-N-(3-methylphenyl)acetamide;
N-(2,3-dimethylphenyl)-2-[3-(4-fluorophenyl)-2-oxo-1,4-diazaspiro[4.4]non-3-en-1-yl]acetamide;
2-[3-(4-fluorophenyl)-2-oxo-1,4-diazaspiro[4.4]non-3-en-1-yl]-N-[4-(methyloxy)phenyl]acetamide;
N-(2,4-dimethylphenyl)-2-[3-(4-fluorophenyl)-2-oxo-1,4-diazaspiro[4.4]non-3-en-1-yl]acetamide;
2-[3-(4-fluorophenyl)-2-oxo-1,4-diazaspiro[4.4]non-3-en-1-yl]-N-(4-methylphenyl)acetamide;
N-(3-fluorophenyl)-2-[3-(4-fluorophenyl)-2-oxo-1,4-diazaspiro[4.4]non-3-en-1-yl]acetamide;
N-(3-chloro-4-fluorophenyl)-2-[3-(4-fluorophenyl)-2-oxo-1,4-diazaspiro[4.4]non-3-en-1-yl]acetamide;
N-(4-chlorophenyl)-2-[3-(4-fluorophenyl)-2-oxo-1,4-diazaspiro[4.4]non-3-en-1-yl]acetamide;
N-(3,4-dimethylphenyl)-2-[3-(4-fluorophenyl)-2-oxo-1,4-diazaspiro[4.4]non-3-en-1-yl]acetamide;
N-(4-fluorophenyl)-2-[3-(4-fluorophenyl)-2-oxo-1,4-diazaspiro[4.4]non-3-en-1-yl]acetamide;
N-[3-(methyloxy)phenyl]-2-(2-oxo-3-phenyl-1,4-diazaspiro[4.4]non-3-en-1-yl)acetamide;
N-(4-fluoro-3-methylphenyl)-2-{3-[4-(methyloxy)phenyl]-2-oxo-1,4-diazaspiro[4.5]dec-3-en-1-yl}acetamide;
N-[2-(methyloxy)phenyl]-2-[3-(4-methylphenyl)-2-oxo-1,4-diazaspiro[4.5]dec-3-en-1-yl]acetamide;
2-[3-(4-chlorophenyl)-2-oxo-1,4-diazaspiro[4.5]dec-3-en-1-yl]-N-(2,3-difluorophenyl)acetamide;
2-[3-(4-chlorophenyl)-2-oxo-1,4-diazaspiro[4.5]dec-3-en-1-yl]-N-(2-methylphenyl)acetamide;
N-(2,4-dimethylphenyl)-2-[3-(4-methylphenyl)-2-oxo-1,4-diazaspiro[4.5]dec-3-en-1-yl]acetamide;
N-(3,4-difluorophenyl)-2-[3-(4-methylphenyl)-2-oxo-1,4-diazaspiro[4.5]dec-3-en-1-yl]acetamide;
N-(3-fluorophenyl)-2-[3-(4-methylphenyl)-2-oxo-1,4-diazaspiro[4.5]dec-3-en-1-yl]acetamide;
N-(2-methylphenyl)-2-[3-(4-methylphenyl)-2-oxo-1,4-diazaspiro[4.5]dec-3-en-1-yl]acetamide;
2-[3-(4-chlorophenyl)-2-oxo-1,4-diazaspiro[4.5]dec-3-en-1-yl]-N-(2,3-dimethylphenyl)acetamide;
2-[3-(4-chlorophenyl)-2-oxo-1,4-diazaspiro[4.5]dec-3-en-1-yl]-N-phenylacetamide;
N-(3-fluorophenyl)-2-{3-[4-(methyloxy)phenyl]-2-oxo-1,4-diazaspiro[4.5]dec-3-en-1-yl}acetamide;
N-(2,4-dimethylphenyl)-2-{3-[4-(methyloxy)phenyl]-2-oxo-1,4-diazaspiro[4.5]dec-3-en-1-yl}acetamide;
N-(3-chlorophenyl)-2-[3-(4-chlorophenyl)-2-oxo-1,4-diazaspiro[4.5]dec-3-en-1-yl]acetamide;
N-(4-chloro-3-methylphenyl)-2-{3-[4-(methyloxy)phenyl]-2-oxo-1,4-diazaspiro[4.5]dec-3-en-1-yl}acetamide; and
2-{3-[4-(methyloxy)phenyl]-2-oxo-1,4-diazaspiro[4.5]dec-3-en-1-yl}-N-(2,3,4-trifluorophenyl)acetamide;
or a pharmaceutically acceptable salts thereof.

21. A method for treating schizophrenia comprising administering therapeutically effective amount of a compound of formula (I) according to claim 15 or a pharmaceutically acceptable salt thereof to a patient in need.

22. A pharmaceutical composition comprising a compound as claimed in claim 15 or a pharmaceutically acceptable salt thereof and at least one pharmaceutically acceptable excipient.

23. A compound selected from the group consisting of:
N-(4-fluoro-3-methylphenyl)-2-{3-[4-(methyloxy)phenyl]-2-oxo-1,4-diazaspiro[4.5]dec-3-en-1-yl}acetamide;
N-[2-(methyloxy)phenyl]-2-[3-(4-methylphenyl)-2-oxo-1,4-diazaspiro[4.5]dec-3-en-1-yl]acetamide;
2-[3-(4-chlorophenyl)-2-oxo-1,4-diazaspiro[4.5]dec-3-en-1-yl]-N-(2,3-difluorophenyl)acetamide;
2-[3-(4-chlorophenyl)-2-oxo-1,4-diazaspiro[4.5]dec-3-en-1-yl]-N-(2-methylphenyl)acetamide;
N-(2,4-dimethylphenyl)-2-[3-(4-methylphenyl)-2-oxo-1,4-diazaspiro[4.5]dec-3-en-1-yl]acetamide;
N-(3,4-difluorophenyl)-2-[3-(4-methylphenyl)-2-oxo-1,4-diazaspiro[4.5]dec-3-en-1-yl]acetamide;
N-(3-fluorophenyl)-2-[3-(4-methylphenyl)-2-oxo-1,4-diazaspiro[4.5]dec-3-en-1-yl]acetamide;
N-(2-methylphenyl)-2-[3-(4-methylphenyl)-2-oxo-1,4-diazaspiro[4.5]dec-3-en-1-yl]acetamide;
2-[3-(4-chlorophenyl)-2-oxo-1,4-diazaspiro[4.5]dec-3-en-1-yl]-N-(2,3-dimethylphenyl)acetamide;
2-[3-(4-chlorophenyl)-2-oxo-1,4-diazaspiro[4.5]dec-3-en-1-yl]-N-phenylacetamide;
N-(3-fluorophenyl)-2-{3-[4-(methyloxy)phenyl]-2-oxo-1,4-diazaspiro[4.5]dec-3-en-1-yl}acetamide;
N-(2,4-dimethylphenyl)-2-{3-[4-(methyloxy)phenyl]-2-oxo-1,4-diazaspiro[4.5]dec-3-en-1-yl}acetamide;
N-(3-chlorophenyl)-2-[3-(4-chlorophenyl)-2-oxo-1,4-diazaspiro[4.5]dec-3-en-1-yl]acetamide;
N-(4-chloro-3-methylphenyl)-2-{3-[4-(methyloxy)phenyl]-2-oxo-1,4-diazaspiro[4.5]dec-3-en-1-yl}acetamide; and
2-{3-[4-(methyloxy)phenyl]-2-oxo-1,4-diazaspiro[4.5]dec-3-en-1-yl}-N-(2,3,4-trifluorophenyl)acetamide;
or a pharmaceutically acceptable salt thereof.

24. A method for treating schizophrenia comprising administering a therapeutically effective amount of a compound of formula (I) according to claim 23 or a pharmaceutically acceptable salt thereof to a patient in need.

25. A pharmaceutical composition comprising a compound as claimed in claim 15 or a pharmaceutically acceptable salt thereof and at least one pharmaceutically acceptable excipient.
